(19) United States
(12) Patent Application Publication
(10) Pub. No.: US 2015/0336881 A1
(43) Pub. Date: Nov. 26, 2015

(54) AMIDE COMPOUND AND USE THEREOF FOR PEST CONTROL

(71) Applicant: Sumitomo Chemical Company, Limited, Tokyo (JP)

(72) Inventors: Ryota MAEHATA, Takarazuka-shi, Hyogo (JP); Hajime MIZUNO, Takarazuka-shi, Hyogo (JP); Chie SHIMIZU, Takarazuka-shi, Hyogo (JP); Yoshihiko NOKURA, Takarazuka-shi, Hyogo (JP)

(21) Appl. No.: 14/409,298

(22) PCT Filed: Jun. 5, 2013
(86) PCT No.: PCT/JP2013/066127

§ 371 Date: Dec. 18, 2014

(30) Foreign Application Priority Data
Jun. 22, 2012 (JP) 2012-140587

(51) Int. Cl. C07C 323/42; C07C 317/40; A01N 41/10; C07C 323/63; A01N 39/00

(52) U.S. Cl. CPC C07C 323/42; C07C 323/63; A01N 39/00; A01N 41/10; C07C 317/40

(57) ABSTRACT

A fused heterocyclic compound represented by formula (1) has an excellent pest control effect.

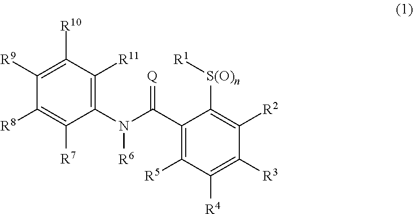

In the formula, R¹, R², R³, R⁴ and R⁵ may be the same or different, and each represents a C₁-C₆ chain hydrocarbon group which may have one or more atoms or groups selected from group X, etc.; R⁶ represents a C₁-C₆ chain hydrocarbon group which may have one or more atoms or groups selected from group W, etc.; R⁷, R⁸, R⁹, R¹⁰, and R¹¹ may be the same or different, and each represents a C₁-C₆ chain hydrocarbon group which may have one or more atoms or groups selected from group X, etc.; Q represents an oxygen atom or a sulfur atom; and each of m and n represents 0, 1 or 2.

### TECHNICAL FIELD

The present invention relates to an amide compound and a use thereof for pest control.

### BACKGROUND ART

WO2003-34671 describes a benzamide derivative used for an agricultural and horticultural agent.

### SUMMARY OF THE INVENTION

The present invention provides a compound having an excellent control effect on pests and a method for controlling pests using the compound.

According to the present invention, an amide compound represented by the following formula (1) has an excellent control effect on pests.

More specifically, the present invention is as described below.

[1] An amide compound represented by formula (1):

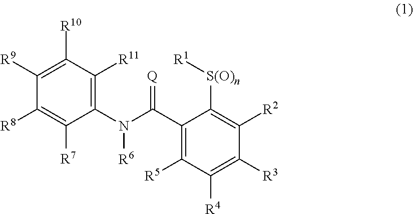

[wherein

R¹ represents a C1 to C6 chain hydrocarbon group optionally having one or more atoms or groups selected from group X or a C3 to C6 cycloalkyl group optionally having one or more atoms or groups selected from group Y,

R², R³, R⁴ and R⁵ are the same or different and represent a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —S(O)₂NR¹²R¹³, —NR¹²R¹³, —NR¹²C(O)R¹³, —NR¹²CO₂R¹³, —NR¹²S(O)₂R¹⁴, —C(O)R¹², —CO₂R¹², —C(O)NR¹²R¹³, —SF₅, a cyano group, a nitro group, a halogen atom, or a hydrogen atom,

R⁶ represents a C1 to C6 chain hydrocarbon group optionally having one or more atoms or groups selected from group W, a C3 to C6 cycloalkyl group optionally having one or more atoms or groups selected from group Y, a C1 to C6 alkyl group having one phenyl group (wherein the phenyl group optionally has one or more atoms or groups selected from group Z), a C1 to C6 alkyl group having one 5- or 6-membered heterocyclic group (wherein the 5- or 6-membered heterocyclic group optionally has one or more atoms or groups selected from group Z), —C(O)R¹², —C(O)OR¹² or —C(O)NR¹²R¹³,

R⁷ and R¹¹ are the same or different and represent a C1 to C6 chain hydrocarbon group optionally having one or more atoms or groups selected from group X, a C3 to C6 cycloalkyl group optionally having one or more atoms or groups selected from group Y, —OR¹⁶, —S(O)ₘR¹⁶, a fluorine atom or a hydrogen atom,

R⁸, R⁹ and R¹⁰ are the same or different and represent a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —S(O)₂NR¹²R¹³, —NR¹²R¹³, —NR¹²C(O)R¹³, —NR¹²CO₂R¹³, —NR¹²S(O)₂R¹⁴, —C(O)R¹², —CO₂R¹⁴, —C(O)NR¹⁵R¹⁶, —SF₅, a cyano group, a nitro group, a halogen atom, or a hydrogen atom,

R¹² and R¹³ are the same or different and represent a C1 to C6 alkyl group optionally having one or more halogen atoms, a phenyl group optionally having one or more atoms or groups selected from group Z, a 5- or 6-membered heterocyclic group optionally having one or more atoms or groups selected from group Z or a hydrogen atom,

R¹⁴ represents a C1 to C6 alkyl group optionally having one or more halogen atoms, a phenyl group optionally having one or more atoms or groups selected from group Z or a 5- or 6-membered heterocyclic group optionally having one or more atoms or groups selected from group Z,

R¹⁵ represents a C1 to C6 alkyl group optionally having one or more halogen atoms or a hydrogen atom,

R¹⁶ represents a C1 to C6 alkyl group optionally having one or more halogen atoms,

Q represents an oxygen atom or a sulfur atom,

m represents 0, 1 or 2, and

n represents 0, 1 or 2;

Group X: a group consisting of C3 to C6 cycloalkyl groups optionally having one or more atoms or groups selected from the group consisting of halogen atoms and C1 to C3 alkyl groups, C1 to C6 alkoxy groups optionally having one or more halogen atoms, C1 to C6 alkylsulfanyl groups optionally having one or more halogen atoms, C1 to C6 alkylsulfinyl groups optionally having one or more halogen atoms, C1 to C6 alkylsulfonyl groups optionally having one or more halogen atoms, and halogen atoms;

Group Y: a group consisting of C1 to C6 alkyl groups optionally having one or more halogen atoms, C1 to C6 alkoxy groups optionally having one or more halogen atoms, C1 to C6 alkylsulfanyl groups optionally having one or more halogen atoms, C1 to C6 alkylsulfinyl groups optionally having one or more halogen atoms, C1 to C6 alkylsulfonyl groups optionally having one or more halogen atoms, and halogen atoms;

Group Z: a group consisting of C1 to C6 alkyl groups optionally having one or more halogen atoms, C1 to C6 alkoxy groups optionally having one or more halogen atoms, C1 to C6 alkylsulfanyl groups optionally having one or more halogen atoms, C1 to C6 alkylsulfinyl groups optionally having one or more halogen atoms, C1 to C6 alkylsulfonyl groups optionally having one or more halogen atoms, C1 to C6 alkylamino groups optionally having one or more halogen atoms, C2 to C8 dialkylamino groups optionally having one or more halogen atoms, C2 to C6 alkylcarbonyl groups optionally having one or more halogen atoms, C2 to C6 alkoxycarbonyl groups optionally having one or more halogen atoms, hydroxy groups, thiol groups, amino groups, cyano groups, nitro groups, and halogen atoms;

Group W: a group consisting of C3 to C6 cycloalkyl groups optionally having one or more atoms or groups selected from the group consisting of halogen atoms and C1 to C3 alkyl groups, C1 to C6 alkoxy groups optionally having one or more halogen atoms, C1 to C6 alkylsulfanyl groups optionally having one or more halogen atoms, C1 to C6 alkylsulfinyl groups optionally having one or more halogen atoms, C1 to C6 alkylsulfonyl groups optionally having one or more halogen atoms, C2 to C6 alkoxycarbonyl groups optionally having one or more halogen atoms, hydroxy groups, cyano groups, and halogen atoms;

wherein R⁸, R⁹ and R¹⁰ do not simultaneously represent a hydrogen atom, when R⁷, R⁸, R⁹ and R¹¹ represent a hydrogen atom, R¹⁰ does not represent a methyl group, and when R⁷, R⁸, R¹⁰ and represent a hydrogen atom, R⁹ does not represent a pyrrolidin-1-yl group, and

when m is 1 or 2 in —S(O)ₘR¹², R¹² does not represent a hydrogen atom.]

or an N-oxide thereof (hereinafter, referred to as the compound of the present invention).

[2] The compound according to [1],

wherein R¹ is a C3 to C6 cycloalkyl group optionally having one or more atoms or groups selected from the group consisting of halogen atoms and C1 to C3 alkyl groups,

a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups),

a C2 to C6 alkenyl group optionally having one or more halogen atoms, or

a C2 to C6 alkynyl group optionally having one or more halogen atoms,

R², R⁴ and R⁵ are the same or different and are a C1 to C6 alkyl group optionally having one or more halogen atoms, —S(O)ₘR¹², a halogen atom, or a hydrogen atom,

R³ is a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —NR¹²R¹³, —NR¹²C(O)R¹³, —NR¹²CO₂R¹³, —NR¹²S(O)₂R¹⁴, —C(O)₂R¹², —CO₂R¹², —C(O)NR¹²R¹³, —SF₅, a cyano group, a nitro group, a halogen atom, or a hydrogen atom,

R⁶ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms, a C2 to C6 alkynyl group optionally having one or more halogen atoms, a C1 to C6 alkyl group having one thiazolyl group (wherein the thiazolyl group optionally has one or more atoms or groups selected from the group consisting of C1 to C3 alkyl groups optionally having a halogen atom, C1 to C3 alkoxy groups optionally having a halogen atom, and halogen atoms), a C1 to C6 alkyl group having one pyridyl group (wherein the pyridyl group optionally has one or more atoms or groups selected from the group consisting of C1 to C3 alkyl groups optionally having a halogen atom, C1 to C3 alkoxy groups optionally having a halogen atom, and halogen atoms), a (C1 to C6 alkoxy)C1 to C6 alkyl group optionally having one or more halogen atoms, —C(O)R¹² or —C(O)OR¹²,

R⁷ and R¹¹ are the same or different and are a C1 to C6 alkyl group optionally having one or more halogen atoms, —OR¹⁶, —S(O)ₘR¹⁶, a fluorine atom or a hydrogen atom,

R⁸, R⁹ and R¹⁰ are the same or different and are a C1 to C6 chain hydrocarbon group optionally having one or more atoms or groups selected from group X, —OR¹², —S(O)ₘR¹², —SF₅, a halogen atom or a hydrogen atom,

R¹² and R¹³ are the same or different and are a C1 to C6 alkyl group optionally having one or more halogen atoms, a phenyl group (wherein the phenyl group optionally has a C1 to C3 alkyl group optionally having one or more halogen atoms or one or more halogen atoms), a 5- or 6-membered heterocyclic group (wherein the 5- or 6-membered heterocyclic group optionally has a C1 to C3 alkyl group optionally having one or more halogen atoms or one or more halogen atoms) or a hydrogen atom,

R¹⁴ is a C1 to C6 alkyl group optionally having one or more halogen atoms, a phenyl group (wherein the phenyl group optionally has a C1 to C3 alkyl group optionally having one or more halogen atoms or one or more halogen atoms) or a 5- or 6-membered heterocyclic group (wherein the 5- or 6-membered heterocyclic group optionally has a C1 to C3 alkyl group optionally having one or more halogen atoms or one or more halogen atoms), and

Q is an oxygen atom.

[3]
The compound according to [1],

wherein R¹ is a C3 to C6 cycloalkyl group optionally having one or more atoms or groups selected from the group consisting of halogen atoms and C1 to C3 alkyl groups, a C2 to C6 alkyl group optionally having one or more halogen atoms or a (C3 to C6 cycloalkyl)C1 to C3 alkyl group optionally having one or more halogen atoms,

R², R⁴ and R⁵ are the same or different and are a halogen atom or a hydrogen atom,

R³ is a C1 to C6 alkyl group optionally having one or more halogen atoms, a C2 to C6 alkenyl group optionally having one or more halogen atoms, a C2 to C6 alkenyl group optionally having one or more halogen atoms, a 5- or 6-membered heterocyclic group (wherein the 5- or 6-membered heterocyclic group optionally has a C1 to C3 alkyl group optionally having one or more halogen atoms or one or more halogen atoms), —OR¹², —S(O)ₘR, —NR¹²R¹³, —NR¹²C(O)R¹³, —NR¹²CO₂R¹³, —NR¹²S(O)₂R¹⁴, —C(O)R¹², —CO₂R¹², —C(O)NR¹²R¹³, a halogen atom, or a hydrogen atom,

R⁶ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms or a C2 to C6 alkynyl group optionally having one or more halogen atoms,

R⁷ and R¹¹ are the same or different and are a fluorine atom or a hydrogen atom,

R⁸, R⁹ and R¹⁰ are the same or different and are a C1 to C6 alkyl group optionally having one or more halogen atoms, a C2 to C6 alkenyl group optionally having one or more halogen atoms, a C2 to C6 alkynyl group optionally having one or more halogen atoms, —OR¹², —S(O)ₘR¹², a halogen atom or a hydrogen atom,

R¹² and R¹³ are the same or different and are a C1 to C6 alkyl group optionally having one or more halogen atoms or a hydrogen atom,

R¹⁴ is a C1 to C6 alkyl group optionally having one or more halogen atoms, and

Q is an oxygen atom.

[4]
The compound as defined in any of [1] to [3], wherein R², R⁴ and R⁵ are a hydrogen atom.
[5]
The compound as defined in any of [1] to [4], wherein R⁷ and R¹¹ are a hydrogen atom.
[6]
The compound according to any of [1] to [5], wherein R³ is a C1 to C3 alkyl group optionally having one or more halogen atoms, a C1 to C3 alkoxy group optionally having one or more halogen atoms, a C1 to C3 alkylsulfanyl group optionally having one or more halogen atoms, a C1 to C3 alkylsulfinyl group optionally having one or more halogen atoms, a C1 to C3 alkylsulfonyl group optionally having one or more halogen atoms, a C1 to C6 alkylamino group optionally having one or more halogen atoms, a C2 to C8 dialkylamino group optionally having one or more halogen atoms, a C2 to C6 alkylcarbonylamino group optionally having one or more halogen atoms, a C2 to C6 alkoxycarbonylamino group optionally having one or more halogen atoms, a C1 to C3 alkylsulfonylamino group optionally having one or more halogen atoms, an amino group, a C2 to C6 alkylcarbonyl group optionally having one or more halogen atoms, a C2 to C6 alkoxycarbonyl group optionally having one or more halogen atoms, a C2 to C6 alkylaminocarbonyl group optionally having one or more halogen atoms, a C3 to C8 dialkylaminocarbonyl group optionally having one or more halogen atoms, an aminocarbonyl group, a pyridyl group (wherein the pyridyl group optionally has one or more atoms or substituents selected from the group consisting of C1 to C3 alkyl groups optionally having a halogen atom, C1 to C3 alkoxy groups optionally having a halogen atom, and halogen atoms), a pyrimidinyl group (wherein the pyrimidinyl group optionally has one or more atoms or substituents selected from the group consisting of C1 to C3 alkyl groups optionally having a halogen atom, C1 to C3 alkoxy groups optionally having a halogen atom, and halogen atoms), a halogen atom, or a hydrogen atom.
[7]
The compound according to any of [1] to [6], wherein R⁶ is a C1 to C6 alkyl group optionally having one or more atoms or groups selected from the group consisting of halogen atoms and a cyclopropyl group.
[8]
The compound according to any of [1] to [7], wherein R¹ is an ethyl group, a cyclopropyl group, or a cyclopropylmethyl group.
[9]
The compound according to any of [1] to [8], wherein R⁸, R⁹ and R¹⁰ are the same or different and are a C1 to C6 haloalkyl group, a C2 to C6 haloalkenyl group, a C1 to C6 haloalkoxy group, a C1 to C6 haloalkylsulfanyl group, a C1 to C6 haloalkylsulfinyl group, a C1 to C6 haloalkylsulfonyl group, a halogen atom, or a hydrogen atom.
[10]
The compound according to any of [1] to [8], wherein R⁸, R⁹ and R¹⁰ are the same or different and are a C1 to C3 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.
[11]
The compound according to any of [1] to [8], wherein R⁷, R⁸, R¹⁰ and R¹¹ are a hydrogen atom, R⁹ is a C1 to C3 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, or a halogen atom.
[12]
The compound according to any of [1] to [8], wherein R⁷, R⁹ and R¹¹ are a hydrogen atom, R⁸ and R¹⁰ are the same or different and are a C1 to C3 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.
[13]
A pest control composition comprising the compound as defined in any of [1] to [12], and an inert carrier.
[14]
A method for controlling pests comprising applying an effective amount of the compound as defined in any of [1] to [12] to a pest or a pest-infested area.

### MODE FOR CARRYING OUT THE INVENTION

In the compound of the present invention, an N-oxide is a compound in which the nitrogen atom constituting the ring on the heterocyclic group is oxidized. Examples of the heterocyclic group that may form the N-oxide include a pyridine ring.

The groups used in the description of the present specification will be described below with examples.

The notation of Ca to Cb chain hydrocarbon group in the present specification represents a straight-chain or branched-chain saturated or unsaturated hydrocarbon group having the number of carbon atoms of a to b.

Examples of the “C1 to C6 chain hydrocarbon group” include C1 to C6 alkyl groups such as a methyl group, an ethyl group, a propyl group, an isopropyl group, a butyl group, an isobutyl group, a sec-butyl group, a tert-butyl group, a pentyl group, a neopentyl group and a hexyl group; C2 to C6 alkenyl groups such as a vinyl group, a 1-propenyl group, a 2-propenyl group, a 1-methylvinyl group, a 2-methyl-1-propenyl group, a 1-butenyl group, a 2-butenyl group, a 3-butenyl group, a 1-pentenyl group and a 1-hexenyl group; and C2 to C6 alkynyl groups such as an ethynyl group, a propargyl group, a 2-butynyl group, a 3-butynyl group, a 1-pentynyl group and a 1-hexynyl group.

The notation of Ca to Cb alkyl group in the present specification represents a straight-chain or branched-chain hydrocarbon group having the number of carbon atoms of a to b.

Examples of the “C1 to C6 alkyl group” include a methyl group, an ethyl group, a propyl group, an isopropyl group, a butyl group, an isobutyl group, a sec-butyl group, a tert-butyl group, a pentyl group, a neopentyl group, and a hexyl group.

The notation of Ca to Cb alkenyl group in the present specification represents a straight-chain or branched-chain unsaturated hydrocarbon group having the number of carbon atoms of a to b, and having one or two or more double bonds in the molecule.

Examples of the “C2 to C6 alkenyl group” include a vinyl group, a 1-propenyl group, a 2-propenyl group, a 1-methylvinyl group, a 2-methyl-1-propenyl group, a 1-butenyl group, a 2-butenyl group, a 3-butenyl group, a 1-pentenyl group, and a 1-hexenyl group.

The notation of Ca to Cb alkynyl group in the present specification represents a straight-chain or branched-chain unsaturated hydrocarbon group having the number of carbon atoms of a to b, and having one or two or more triple bonds in the molecule.

Examples of the “C2 to C6 alkynyl group” include an ethynyl group, a propargyl group, a 2-butynyl group, a 3-butynyl group, a 1-pentynyl group, and a 1-hexynyl group.

The notation of Ca to Cb cycloalkyl group in the present specification represents a cyclic alkyl group having the number of carbon atoms of a to b.

The “C3 to C6 cycloalkyl group” includes a cyclopropyl group, a cyclobutyl group, a cyclopentyl group, and a cyclohexyl group.

The notation of “Ca to Cb alkoxy group” in the present specification represents a straight-chain or branched-chain alkyl —O— group having the number of carbon atoms of a to b.

Examples of the “C1 to C6 alkoxy group” include a methoxy group, an ethoxy group, a propoxy group, an isopropoxy group, a butoxy group, an isobutoxy group, a sec-butoxy group, a tert-butoxy group, a pentyloxy group, a neopentyloxy group, and a hexyloxy group.

The notation of Ca to Cb alkylsulfanyl group in the present specification represents a straight-chain or branched-chain alkyl —S— group having the number of carbon atoms of a to b.

Examples of the “C1 to C6 alkylsulfanyl group” include a methylsulfanyl group, an ethylsulfanyl group, a propylsulfanyl group, an isopropylsulfanyl group, a butylsulfanyl group, a pentylsulfanyl group, and a hexylsulfanyl group.

The notation of Ca to Cb alkylsulfinyl group in the present specification represents a straight-chain or branched-chain alkyl —S(O)— group having the number of carbon atoms of a to b.

Examples of the “C1 to C6 alkylsulfinyl group” include a methylsulfinyl group, an ethylsulfinyl group, a propylsulfinyl group, an isopropylsulfinyl group, a butylsulfinyl group, a pentylsulfinyl group, and a hexylsulfinyl group.

The notation of Ca to Cb alkylsulfonyl group in the present specification represents a straight-chain or branched-chain alkyl —S(O)₂— group having the number of carbon atoms of a to b.

Examples of the “C1 to C6 alkylsulfonyl group” include a methylsulfonyl group, an ethylsulfonyl group, a propylsulfonyl group, an isopropylsulfonyl group, a butylsulfonyl group, a pentylsulfonyl group, and a hexylsulfonyl group.

The notation of Ca to Cb alkylamino group in the present specification represents a straight-chain or branched-chain alkyl —NH— group having the number of carbon atoms of a to b.

Examples of the “C1 to C6 alkylamino group” include a methylamino group, an ethylamino group, a propylamino group, an isopropylamino group, and a butylamino group.

The notation of Ca to Cb dialkylamino group in the present specification represents a straight-chain or branched-chain dialkylamino group having a total number of carbon atoms of each alkyl group of a to b, in which the number of carbon atoms of each alkyl group may be the same or different.

Examples of the “C2 to C8 dialkylamino group” include a dimethylamino group, a diethylamino group, and a dipropylamino group.

The notation of Ca to Cb alkylcarbonyl group in the present specification represents a straight-chain or branched-chain alkyl —C(O)— group having the number of carbon atoms of a to b.

Examples of the “C2 to C6 alkylcarbonyl group” include an acetyl group, a propionyl group, a butyryl group, a pentanoyl group, and a hexanoyl group.

The notation of Ca to Cb alkoxycarbonyl group in the present specification represents a straight-chain or branched-chain alkyl —O—C(O)— group having the number of carbon atoms of a to b.

Examples of the “C2 to C6 alkoxycarbonyl group” include a methoxycarbonyl group, an ethoxycarbonyl group, a propoxycarbonyl group, a butoxycarbonyl group, a pentyloxycarbonyl group, and a tert-butoxycarbonyl group.

The notation of (Ca to Cb alkoxy)Cc to Cd alkyl group in the present specification represents a Cc to Cd alkyl group in which a hydrogen atom bound to the carbon atom of the Cc to Cd alkyl group is substituted by a Ca to Cb alkoxy group. The numbers of carbon atoms of the alkoxy group and the alkyl group are the same or different, and the notation represents a straight-chain or branched-chain alkyl —O— alkyl group.

The “(C1 to C6 alkoxy) C1 to C6 alkyl group” in the compound of the present invention is a C1 to C6 alkyl group combining with a C1 to C6 alkoxy group, and the number of carbon atoms of the (C1 to C6 alkoxy)C1 to C6 alkyl group is 2 to 12.

Examples of the “(C1 to C6 alkoxy)C1 to C6 alkyl group” include a methoxymethyl group, an ethoxymethyl group, a 1-(methoxy)ethyl group, a 2-(methoxy)ethyl group, a 1-(ethoxy)ethyl group, and a 2-(ethoxy)ethyl group.

The notation of (Ca to Cb cycloalkyl)Cc to Cd alkyl group in the present specification represents a Cc to Cd alkyl group in which a hydrogen atom bound to the carbon atom of the Cc to Cd alkyl group is substituted by a Ca to Cb cycloalkyl group. The numbers of carbon atoms of the cycloalkyl group and the alkyl group may be the same or different, and the notation represents a cyclic alkyl-alkyl group.

The “(C3 to C6 cycloalkyl)C1 to C3 alkyl group” in the compound of the present invention is a C1 to C3 alkyl group combining with a C3 to C6 cycloalkyl group, and the number of carbon atoms of the (C3 to C6 cycloalkyl)C1 to C3 alkyl group is 4 to 9.

Examples of the “(C3 to C6 cycloalkyl)C1 to C3 alkyl group” include a cyclopropylmethyl group, a 2-cyclopropylethyl group, and a 1-cyclopropylethyl group.

The notation of Ca to Cb haloalkyl group in the present specification represents a straight-chain or branched-chain hydrocarbon group having the number of carbon atoms of a to b, in which one or more hydrogen atoms bound to the carbon atom are substituted by a halogen atom, and at that time, when having two or more halogen atoms, those halogen atoms may be the same or different from each other.

Examples of the “C1 to C6 haloalkyl group” include a fluoromethyl group, a chloromethyl group, a bromomethyl group, an iodomethyl group, a difluoromethyl group, a dichloromethyl group, a trifluoromethyl group, a chlorodifluoromethyl group, a bromodifluoromethyl group, a trichloromethyl group, a 2-fluoroethyl group, a 2-chloroethyl group, a 2-bromoethyl group, a 2,2-difluoroethyl group, a 2,2,2-trifluoroethyl group, a pentafluoroethyl group, a heptafluoropropyl group, and a heptafluoroisopropyl group.

The notation of Ca to Cb haloalkenyl group in the present specification represents a straight-chain or branched-chain unsaturated hydrocarbon group having the number of carbon atoms of a to b, in which one or more hydrogen atoms bound to the carbon atom are substituted by a halogen atom, and having one or two or more double bonds in the molecule and at that time, when having two or more halogen atoms, those halogen atoms may be the same or different from each other.

Examples of the “C2 to C6 haloalkenyl group” include a 3,3-dichloro-2-propenyl group, a 3,3-dibromo-2-propenyl group, and the like.

The notation of Ca to Cb haloalkoxy in the present specification represents a straight-chain or branched-chain alkyl —O— group having the number of carbon atoms of a to b, in which one or more hydrogen atoms bound to the carbon atom are substituted by a halogen atom, and at that time, when having two or more halogen atoms, those halogen atoms may be the same or different from each other.

Examples of the “C1 to C6 haloalkoxy group” include a trifluoromethoxy group, a 2,2,2-trifluoroethoxy group, a 2,2,2-trichoroethoxy group, a 2,2,3,3-tetrafluoropropoxy group, and a 2,2,3,4,4,4-hexa fluorobutoxy group.

The notation of Ca to Cb haloalkylsulfanyl in the present specification represents a straight-chain or branched-chain alkyl —S— group having the number of carbon atoms of a to b, in which one or more hydrogen atoms bound to the carbon atom are substituted by a halogen atom, and at that time, when having two or more halogen atoms, those halogen atoms may be the same or different from each other.

Examples of the “C1 to C6 haloalkylsulfanyl group” include a trifluoromethylsulfanyl group, a 2,2,2-trifluoroethylsulfanyl group, a 2,2,2-trichoroethylsulfanyl group, a 2,2,3,3-tetrafluoropropylsulfanyl group, and a 2,2,3,4,4,4-hexafluorobutylsulfanyl group.

The notation of Ca to Cb haloalkylsulfinyl in the present specification represents a straight-chain or branched-chain alkyl —S(O)— group having the number of carbon atoms of a to b, in which one or more hydrogen atoms bound to the carbon atom are substituted by a halogen atom, and at that time, when having two or more halogen atoms, those halogen atoms may be the same or different from each other.

Examples of the “C1 to C6 haloalkylsulfinyl group” include a trifluoromethylsulfinyl group, a 2,2,2-trifluoroethylsulfinyl group, a 2,2,2-trichoroethylsulfinyl group, a 2,2,3,3-tetrafluoropropylsulfinyl group, and a 2,2,3,4,4,4-hexafluorobutylsulfinyl group.

The notation of Ca to Cb haloalkylsulfonyl in the present specification represents a straight-chain or branched-chain alkyl —S(O)₂— group having the number of carbon atoms of a to b, in which one or more hydrogen atoms bound to the carbon atom are substituted by a halogen atom, and at that time, when having two or more halogen atoms, those halogen atoms may be the same or different from each other.

Examples of the “C1 to C6 haloalkylsulfonyl group” include a trifluoromethylsulfony group, a 2,2,2-trifluoroethylsulfonyl group, a 2,2,2-trichoroethylsulfonyl group, a 2,2,3,3-tetrafluoropropylsulfonyl group, and a 2,2,3,4,4,4-hexafluorobutylsulfonyl group.

The notation of Ca to Cb perfluoroalkyl in the present specification represents a straight or branched alkyl group having the number of carbon atoms of a to b, in which all hydrogen atoms bound to the carbon atom are substituted by a fluorine atom.

Examples of the “C1 to C3 perfluoroalkyl group” include a trifluoromethyl group, a pentafluoroethyl group, a heptafluoropropyl group, and a heptafluoroisopropyl group.

The notation of Ca to Cb perfluoroalkoxy in the present specification represents a straight-chain or branched-chain alkyl —O— group having the number of carbon atoms of a to b, in which all hydrogen atoms bound to the carbon atom are substituted by a fluorine atom.

Examples of the “C1 to C3 perfluoroalkoxy group” include a trifluoromethoxy group, a pentafluoroethoxy group, a heptafluoropropoxy group, and a heptafluoroisopropoxy group.

The notation of Ca to Cb perfluoroalkylsulfanyl in the present specification represents a straight-chain or branched-chain alkyl —S— group having the number of carbon atoms of a to b, in which all hydrogen atoms bound to the carbon atom are substituted by a fluorine atom.

Examples of the “C1 to C3 perfluoroalkylsulfanyl group” include a trifluoromethylsulfanyl group, a pentafluoroethylsulfanyl group, a heptafluoropropylsulfanyl group, and a heptafluoroisopropylsulfanyl group.

The notation of Ca to Cb perfluoroalkylsulfinyl in the present specification represents a straight-chain or branched-chain alkyl —S(O)— group having the number of carbon atoms of a to b, in which all hydrogen atoms bound to the carbon atom are substituted by a fluorine atom.

Examples of the “C1 to C3 perfluoroalkylsulfinyl group” include a trifluoromethylsulfinyl group, a pentafluoroethylsulfinyl group, a heptafluoropropylsulfinyl group, and a heptafluoroisopropylsulfinyl group.

The notation of Ca to Cb perfluoroalkylsulfonyl in the present specification represents a straight-chain or branched-chain alkyl —S(O)₂— group having the number of carbon atoms of a to b, in which all hydrogen atoms bound to the carbon atom are substituted by a fluorine atom.

Examples of the “C1 to C3 perfluoroalkylsulfonyl group” include a trifluoromethylsulfonyl group, a pentafluoroethylsulfonyl group, a heptafluoropropylsulfonyl group, and a heptafluoroisopropylsulfonyl group.

In the notation of “optionally having one or more atoms or groups selected from group X” in the present specification, when having two or more atoms or groups selected from group X, the atoms or groups selected from the group X may be the same or different from each other.

In the notation of “optionally having one or more atoms or groups selected from group Y” in the present specification, when having two or more atoms or groups selected from group Y, the atoms or groups selected from the group Y may be the same or different from each other.

In the notation of “optionally having one or more atoms or groups selected from group Z” in the present specification, when having two or more atoms or groups selected from group Z, the atoms or groups selected from the group Z may be the same or different from each other.

In the notation of “optionally having one or more atoms or groups selected from group W” in the present specification, when having two or more atoms or groups selected from group W, the atoms or groups selected from the group W may be the same or different from each other.

In the notation of “optionally having one or more halogen atoms” in the present specification, when having two or more halogen atoms, those halogen atoms may be the same or different from each other.

In the notation of “optionally having one or more atoms or groups selected from the group consisting of halogen atoms and C1 to C3 alkyl groups” in the present specification, when having two or more atoms or groups, the atoms or groups may be the same or different from each other.

The “halogen atom” in the compound of the present invention refers to a fluorine atom, a chlorine atom, a bromine atom, and an iodine atom.

The notation of the “C1 to C6 chain hydrocarbon group optionally having one or more atoms or groups selected from group X” in the compound of the present invention represents a straight-chain or branched-chain hydrocarbon group comprising a carbon atom number of 1 to 6, in which a hydrogen atom bound to the carbon atom is optionally substituted by an atom or group selected from group X, and at that time, when having two or more atoms or groups selected from group X, the atoms or groups selected from group X may be the same or different from each other.

Examples of the “C1 to C6 chain hydrocarbon group optionally having one or more atoms or groups selected from group X” include C1 to C6 alkyl groups optionally having one or more atoms or groups selected from group X such as a methyl group, an ethyl group, a propyl group, an isopropyl group, a butyl group, an isobutyl group, a sec-butyl group, a tert-butyl group, a pentyl group, a neopentyl group, a hexyl group, a methoxymethyl group, an ethoxymethyl group, a propoxymethyl group, an isopropoxymethyl group, a butoxymethyl group, a sec-butoxymethyl group, a tert-butoxymethyl group, a 2-methoxyethyl group, a 2-ethoxyethyl group, a 2-propoxyethyl group, a 2-isopropoxyethyl group, a 2-butoxyethyl group, a 2-sec-butoxyethyl group, a 2-tert-butoxyethyl group, a trifluoromethyl group, a trichloromethyl group, a 2-fluoroethyl group, a 2,2-difluoroethyl group, a 2,2,2-tri fluoroethyl group, a pentafluoroethyl group, a methylsulfanylethyl group, an ethylsulfanylethyl group, a methylsulfinylethyl group, a methylsulfonylethyl group, a cyclopropylmethyl group, a 1-methylcyclopropylmethyl group, and a 2,2-difluorocyclopropylmethyl group; C2 to C6 alkenyl groups optionally having one or more atoms or groups selected from group X such as a vinyl group, a 1-propenyl group, a 2-propenyl group, a 1-methylvinyl group, a 2-methyl-1-propenyl group, a 1-butenyl group, a 2-butenyl group, a 3-butenyl group, a 1-pentenyl group, a 1-hexenyl group, a 1,1-difluoroallyl group and a pentafluoroallyl group; and C2 to C6 alkynyl groups optionally having one or more atoms or groups selected from group X such as an ethynyl group, a propargyl group, a 2-butynyl group, a 3-butynyl group, a 1-pentynyl group, a 1-hexynyl group, a 4,4,4-trifluoro-2-butynyl group, and the C1 to C6 chain hydrocarbon group optionally having one or more atoms or groups selected from group X is selected in the range of each specified number of carbon atoms.

The notation of the “C3 to C6 cycloalkyl group optionally having one or more atoms or groups selected from group Y” in the compound of the present invention represents a cyclic alkyl group comprising a carbon atom number of 3 to 6, in which a hydrogen atom bound to the carbon atom is optionally substituted by an atom or group selected from group Y, and at that time, when having two or more atoms or groups selected from group Y, the atoms or groups selected from group Y may be the same or different from each other.

Examples of the “C3 to C6 cycloalkyl group optionally having one or more atoms or groups selected from group Y” include a cyclopropyl group, a cyclobutyl group, a cyclopentyl group, a cyclohexyl group, a 1-methylcyclohexyl group, a 2-methylcyclohexyl group, a 3-methylcyclohexyl group, a 4-methylcyclohexyl group, a 2-methoxylcyclohexyl group, a 3-methoxylcyclohexyl group, a 4-methoxylcyclohexyl group, a 1-fluorocyclohexyl group, a 2-fluorocyclohexyl group, a 3-fluorocyclohexyl group, and a 4-fluorocyclohexyl group.

Examples of the “C1 to C6 chain hydrocarbon group optionally having one or more atoms or groups selected from group W” in the compound of the present invention include C1 to C6 alkyl groups optionally having one or more atoms or groups selected from group W such as a methyl group, an ethyl group, a propyl group, an isopropyl group, a butyl group, an isobutyl group, a sec-butyl group, a tert-butyl group, a pentyl group, a neopentyl group, a hexyl group, a trifluoromethyl group, a trichloromethyl group, a 2-fluoroethyl group, a 2,2-difluoroethyl group, a 2,2,2-trifluoroethyl group, a pentafluoroethyl group, a methoxymethyl group, an ethoxymethyl group, a propoxymethyl group, an isopropoxymethyl group, a butoxymethyl group, a sec-butoxymethyl group, an isobutoxymethyl group, a tert-butoxymethyl group, a methoxyethyl group, an ethoxyethyl group, a propoxyethyl group, an isopropoxyethyl group, a butoxyethyl group, a sec-butoxyethyl group, an isobutoxyethyl group, a tert-butoxyethyl group, a methylsulfanylethyl group, an ethylsulfanylethyl group, a methylsulfinylethyl group, a methylsulfonylethyl group, a methoxycarbonylmethyl group, a methoxycarbonylethyl group, a 2-cyanoethyl group, a propyl-2-one group, a cyclopropylmethyl group, and a cyclopropylmethyl group; C2 to C6 alkenyl groups optionally having one or more atoms or groups selected from group W such as a vinyl group, a 1-propenyl group, a 2-propenyl group, a 1-methylvinyl group, a 2-methyl-1-propenyl group, a 1-butenyl group, a 2-butenyl group, a 3-butenyl group, a 1-pentenyl group, a 1-hexenyl group, a 1,1-difluoroallyl group and a pentafluoroallyl group; and C2 to C6 alkynyl groups optionally having one or more atoms or groups selected from group W such as an ethynyl group, a propargyl group, a 2-butynyl group, a 3-butynyl group, a 1-pentynyl group, a 1-hexynyl group, and a 4,4,4-trifluoro-2-butynyl group. At that time, when having two or more atoms or groups selected from group W, the atoms or groups selected from group W may be the same or different from each other.

Examples of the “C1 to C6 alkyl group optionally having one or more halogen atoms” in the compound of the present invention include a methyl group, an ethyl group, a propyl group, an isopropyl group, a butyl group, an isobutyl group, a sec-butyl group, a tert-butyl group, a pentyl group, a neopentyl group, a hexyl group, a fluoromethyl group, a chloromethyl group, a bromomethyl group, an iodomethyl group, a difluoromethyl group, a dichloromethyl group, a trifluoromethyl group, a chlorodifluoromethyl group, a bromodifluoromethyl group, a trichloromethyl group, a 2-fluoroethyl group, a 2-chloroethyl group, a 2-bromoethyl group, a 2,2-difluoroethyl group, a 2,2,2-trifluoroethyl group, a pentafluoroethyl group, a heptafluoropropyl group, and a heptafluoroisopropyl group.

Examples of the C2 to C6 alkyl groups optionally having one or more halogen atoms” in the compound of the present invention include an ethyl group, a propyl group, an isopropyl group, a butyl group, an isobutyl group, a sec-butyl group, a tert-butyl group, a pentyl group, a neopentyl group, a hexyl group, a 2-fluoroethyl group, a 2-chloroethyl group, a 2-bromoethyl group, a 2,2-difluoroethyl group, a 2,2,2-trifluoroethyl group, a pentafluoroethyl group, a heptafluoropropyl group, and a heptafluoroisopropyl group.

Examples of the “C1 to C3 alkyl groups optionally having one or more halogen atoms” in the compound of the present invention include a methyl group, an ethyl group, a propyl group, an isopropyl group, a trifluoromethyl group, a trichloromethyl group, a 2,2-difluoroethyl group, and a 2,2,2-trifluoroethyl group.

Examples of the “C2 to C6 alkenyl group optionally having one or more halogen atoms” in the compound of the present invention include a vinyl group, a 1-propenyl group, a 2-propenyl group, a 1-methylvinyl group, a 2-methyl-1-propenyl group, a 1-butenyl group, a 2-butenyl group, a 3-butenyl group, a 1-pentenyl group, a 1-hexenyl group, a 1,1-difluoroallyl group, and a pentafluoroallyl group.

Examples of the “C2 to C6 alkynyl group optionally having one or more halogen atoms” in the compound of the present invention include an ethynyl group, a propargyl group, a 2-butynyl group, a 3-butynyl group, a 1-pentynyl group, a 1-hexynyl group, and a 4,4,4-trifluoro-2-butynyl group.

The notation of the “a (C1 to C6 alkoxy) C1 to C6 alkyl group optionally having one or more halogen atoms” in the compound of the present invention represents a C1 to C6 alkyl group having one C1 to C6 alkoxy group optionally having one or more halogen atoms, and when having two or more halogen atoms, those halogen atoms may be the same or different from each other. Examples include a methoxymethyl group, an ethoxymethyl group, a 1-(methoxy)ethyl group, a 2-(methoxy)ethyl group, a 1-(ethoxy)ethyl group, a 2-(ethoxy)ethyl group, and a (2,2,2-trifluoroethoxy)methyl group.

Examples of the “C3 to C6 cycloalkyl groups optionally having one or more atoms or groups selected from the group consisting of halogen atoms and C1 to C3 alkyl groups” in the compound of the present invention include a cyclopropyl group, a 1-methylcyclopropyl group, a 2-methylcyclopropyl group, a 1-fluorocyclopropyl group, a 2,2-difluorocyclopropyl group, a 2,2-dichlorocyclopropyl group, a 2,2-dibromocyclopropyl group, a cyclobutyl group, a cyclopentyl group, and a cyclohexyl group.

Examples of the “C1 to C6 alkoxy groups optionally having one or more halogen atoms” in the compound of the present invention include a methoxy group, a trifluoromethoxy group, an ethoxy group, a 2,2,2-trifluoroethoxy group, a propoxy group, an isopropoxy group, a butoxy group, an isobutoxy group, a sec-butoxy group, a tert-butoxy group, a pentyloxy group, and a hexyloxy group.

Examples of the “C1 to C3 alkoxy groups optionally having one or more halogen atoms” in the compound of the present invention include a methoxy group, a trifluoromethoxy group, an ethoxy group, a 2,2,2-trifluoroethoxy group, a propoxy group, and an isopropoxy group.

Examples of the “C1 to C6 alkylsulfanyl groups optionally having one or more halogen atoms” in the compound of the present invention include a methylsulfanyl group, an ethylsulfanyl group, a propylsulfanyl group, an isopropylsulfanyl group, a butylsulfanyl group, a pentylsulfanyl group, a hexylsulfanyl group, a trifluoromethylsulfanyl group, a 2,2,2-trifluoroethylsulfanyl group, and a pentafluoroethylsulfanyl group.

Examples of the “C1 to C3 alkylsulfanyl groups optionally having one or more halogen atoms” in the compound of the present invention include a methylsulfanyl group, an ethylsulfanyl group, a propylsulfanyl group, an isopropylsulfanyl group, a trifluoromethylsulfanyl group, and a 2,2,2-trifluoroethylsulfanyl group.

Examples of the “C1 to C6 alkylsulfinyl groups optionally having one or more halogen atoms” in the compound of the present invention include a methylsulfinyl group, an ethylsulfinyl group, a propylsulfinyl group, an isopropylsulfinyl group, a butylsulfinyl group, a pentylsulfinyl group, a hexylsulfinyl group, a trifluoromethylsulfinyl group, a 2,2,2-trifluoroethylsulfinyl group, and a pentafluoroethylsulfinyl group.

Examples of the “C1 to C3 alkylsulfinyl groups optionally having one or more halogen atoms” in the compound of the present invention include a methylsulfinyl group, an ethylsulfinyl group, a propylsulfinyl group, an isopropylsulfinyl group, a trifluoromethylsulfinyl group, and a 2,2,2-trifluoroethylsulfinyl group.

Examples of the “C1 to C6 alkylsulfonyl groups optionally having one or more halogen atoms” in the compound of the present invention include a methylsulfonyl group, an ethylsulfonyl group, a propylsulfonyl group, an isopropylsulfonyl group, a butylsulfonyl group, a pentylsulfonyl group, a hexylsulfonyl group, a trifluoromethylsulfonyl group, a 2,2,2-trifluoroethylsulfonyl group, and a pentafluoroethylsulfonyl group.

Examples of the “C1 to C3 alkylsulfonyl groups optionally having one or more halogen atoms” in the compound of the present invention include a methylsulfonyl group, an ethylsulfonyl group, a propylsulfonyl group, an isopropylsulfonyl group, a trifluoromethylsulfinyl group, and a 2,2,2-trifluoroethylsulfonyl group.

Examples of the “C1 to C6 alkylamino groups optionally having one or more halogen atoms” in the compound of the present invention include a methylamino group, an ethylamino group, a 2,2,2-trifluoroethylamino group, a propylamino group, an isopropylamino group, and a butylamino group.

Examples of the “C2 to C8 dialkylamino groups optionally having one or more halogen atoms” in the compound of the present invention include a dimethylamino group, a diethylamino group, a bis(2,2,2-trifluoroethyl)amino group, and a dipropylamino group.

Examples of the “C2 to C6 alkylcarbonylamino groups optionally having one or more halogen atoms” in the compound of the present invention include an acetylamino group, a propionylamino group, a butyrylamino group, a pentanoylamino group, a hexanoylamino group, and a trifluoroacetylamino group.

Examples of the “C2 to C6 alkoxycarbonylamino groups optionally having one or more halogen atoms” in the compound of the present invention include a methoxycarbonylamino group, an ethoxycarbonylamino group, a propoxycarbonylamino group, a butoxycarbonylamino group, a pentyloxycarbonylamino group, a tert-butoxycarbonylamino group, and a 2,2,2-trifluoroethoxycarbonylamino group.

Examples of the “C1 to C3 alkylsulfonylamino groups optionally having one or more halogen atoms” in the compound of the present invention include a methylsulfonylamino group, an ethylsulfonylamino group, a propylsulfonylamino group, an isopropylsulfonylamino group, a trifluoromethylsulfinylamino group, and a 2,2,2-trifluoroethylsulfonylamino group.

Examples of the “C2 to C6 alkylcarbonyl groups optionally having one or more halogen atoms” in the compound of the present invention include an acetyl group, a propionyl group, a butyryl group, a pentanoyl group, a hexanoyl group, and a trifluoroacetyl group.

Examples of the “C2 to C6 alkoxycarbonyl groups optionally having one or more halogen atoms” in the compound of the present invention include a methoxycarbonyl group, an ethoxycarbonyl group, a propoxycarbonyl group, a butoxycarbonyl group, a pentyloxycarbonyl group, a tert-butoxycarbonyl group, and a 2,2,2-trifluoroethoxycarbonyl group.

Examples of the “C1 to C6 alkylaminocarbonyl groups optionally having one or more halogen atoms” in the compound of the present invention include a methylaminocarbonyl group, an ethylaminocarbonyl group, a 2,2,2-tri fluoroethyl aminocarbonyl group, a propylaminocarbonyl group, an isopropylaminocarbonyl group, and a butyl aminocarbonyl group.

Examples of the “C3 to C8 dialkylaminocarbonyl groups optionally having one or more halogen atoms” in the compound of the present invention include a dimethylaminocarbonyl group, a diethylaminocarbonyl group, a bis(2,2,2-tri fluoroethyl)aminocarbonyl group, and a dipropylaminocarbonyl group.

Examples of the “C1 to C6 alkyl group optionally having one or more atoms or groups selected from the group consisting of halogen atoms and a cyclolpropyl group (wherein the cyclolpropyl group may have one or more halogen atoms or one or more C1 to C3 alkyl groups)” in the compound of the present invention include a methyl group, an ethyl group, a propyl group, an isopropyl group, a butyl group, an isobutyl group, a sec-butyl group, a tert-butyl group, a pentyl group, a neopentyl group, a hexyl group, a trifluoromethyl group, a trichloromethyl group, a 2-fluoroethyl group, a 2,2-difluoroethyl group, a 2,2,2,-trifluoroethyl group, a pentafluoroethyl group, a heptafluoroisopropyl group, a cyclopropylmethyl group, a 2-cyclopropylethyl group, a 1-cyclopropylethyl group, a 1-methylcyclopropylmethyl group, a 2,2-dimethylcyclopropylmethyl group, and a 2,2-difluorocyclopropylmethyl group.

Examples of the “C2 to C6 alkyl group optionally having one or more atoms or groups selected from the group consisting of halogen atoms and a cyclolpropyl group (wherein the cyclolpropyl group may have one or more halogen atoms or one or more C1 to C3 alkyl groups)” in the compound of the present invention include an ethyl group, a propyl group, an isopropyl group, a butyl group, an isobutyl group, a sec-butyl group, a tert-butyl group, a pentyl group, a neopentyl group, a hexyl group, a 2-fluoroethyl group, a 2,2-difluoroethyl group, a 2,2,2,-trifluoroethyl group, a pentafluoroethyl group, a heptafluoroisopropyl group, a 2-cyclopropylethyl group, a 1-cyclopropylethyl group, and a 2-(2,2-difluorocyclopropyl)ethyl group.

Examples of the “(C3 to C6 cycloalkylalkyl)C1 to C3 alkyl groups optionally having one or more halogen atoms” in the compound of the present invention include a cyclopropylmethyl group, a 2-cyclopropylethyl group, a 1-cyclopropylethyl group, and a 2,2-difluorocyclopropylmethyl group.

Examples of the “C3 to C6 cycloalkyl group optionally having one or more atoms or groups selected from the group consisting of halogen atoms and C1 to C3 alkyl groups” in the compound of the present invention include a cyclopropyl group, a cyclobutyl group, a cyclopentyl group, a cyclohexyl group, a 1-methylcyclohexyl group, a 2-methylcyclohexyl group, a 3-methylcyclohexyl group, a 4-methylcyclohexyl group, a 1-fluorocyclohexyl group, a 2-fluorocyclohexyl group, a 3-fluorocyclohexyl group, and a 4-fluorocyclohexyl group.

The notation of the “phenyl group optionally having one or more atoms or groups selected from group Z” in the compound of the present invention represents a phenyl group in which a hydrogen atom bound to the carbon atom is optionally substituted by an atom or group selected from group Z, and at that time, when having two or more atoms or groups selected from group Z, the atoms or groups selected from group Z may be the same or different from each other.

Examples of the “phenyl group optionally having one or more atoms or groups selected from group Z” include a phenyl group, a 2-fluorophenyl group, a 3-fluorophenyl group, a 4-fluorophenyl group, a 2,3-difluorophenyl group, a 2,4-difluorophenyl group, a 2,5-difluorophenyl group, a 2,6-difluorophenyl group, a 3,4-difluorophenyl group, a 3,5-difluorophenyl group, a 2,3,4,5,6-pentafluorophenyl group, a 2-chlorophenyl group, a 3-chlorophenyl group, a 4-chlorophenyl group, a 2-bromophenyl group, a 3-bromophenyl group, a 4-bromophenyl group, a 2-iodophenyl group, a 3-iodophenyl group, a 4-iodophenyl group, a 2-trifluoromethylphenyl group, a 3-trifluoromethylphenyl group, a 4-trifluoromethylphenyl group, a 2-trifluoromethoxyphenyl group, a 3-trifluoromethoxyphenyl group, a 4-trifluoromethoxyphenyl group, a 2-trifluoromethylsulfanylphenyl group, a 3-trifluoromethylsulfanylphenyl group, a 4-trifluoromethylsulfanylphenyl group, a 4-methoxycarbonylphenyl group, a 4-nitrophenyl group, a 4-cyanophenyl group, a 4-methylaminophenyl group, a 4-dimethylaminophenyl group, a 4-methylsulfinylphenyl group, a 4-methylsulfonylphenyl group, a 4-acetylphenyl group, and a 4-methoxycarbonylphenyl group.

The “heterocyclic group” in the compound of the present invention represents a heterocyclic compound residue containing one or more nitrogen atoms, oxygen atoms or sulfur atoms, other than carbon atoms, as ring-constituting atoms, in the ring structure. In addition, in the compound of the present invention, a 5-membered heterocyclic group means a 5-membered aromatic heterocyclic group or a 5-membered nonaromatic heterocyclic group, and a 6-membered heterocyclic group means a 6-membered aromatic heterocyclic group or a 6-membered nonaromatic heterocyclic group.

The “heterocyclic group” in the “5- or 6-membered heterocyclic group optionally having one or more atoms or groups selected from group Z” in the compound of the present invention represents a heterocyclic compound residue containing one or more nitrogen atoms, oxygen atoms or sulfur atoms, other than carbon atoms, in the ring structure, and at that time, when having two or more atoms or groups selected from group Z, the atoms or groups selected from group Z may be the same or different from each other.

In addition, in the compound of the present invention, a 5- or 6-membered heterocyclic group means a 5- or 6-membered aromatic heterocyclic group or a 5- or 6-membered nonaromatic heterocyclic group.

Examples of the “5- or 6-membered heterocyclic group optionally having one or more atoms or groups selected from group Z” include 5- or 6-membered nonaromatic heterocyclic groups optionally having one or more atoms or groups selected from group Z such as a pyrrolidin-1-yl group, a 3,3,4,4-tetrafluoropyrrolidin-1-yl group, a tetrahydrofuran-2-yl group, a piperidyl group, a morpholinyl group and a thiomorpholinyl group; and 5- or 6-membered aromatic heterocyclic groups optionally having one or more atoms or groups selected from group Z such as a 2-pyrrolyl group, a 2-furyl group, a 3-furyl group, a 5-pyrazolyl group, a 4-pyrazolyl group, a 1-pyrrolyl group, a 1-methyl-2-pyrrolyl group, a 2-methylsulfanyl-1-pyrrolyl group, a 2-methylsulfinyl-1-pyrrolyl group, a 2-methylsulfonyl-1-pyrrolyl group, a 2-methylamino-1-pyrrolyl group, a 2-dimethylamino-1-pyrrolyl group, a 5-bromo-2-furyl group, a 5-nitro-2-furyl group, a 5-cyano-2-furyl group, a 5-methoxy-2-furyl group, a 5-acetyl-2-furyl group, a 5-methoxycarbonyl-2-furyl group, a 2-methyl-3-furyl group, a 2,5-dimethyl-3-furyl group, a 2,4-dimethyl-3-furyl group, a 5-methyl-2-thienyl group, a 3-methyl-2-thienyl group, a 1-methyl-3-trifluoromethyl-5-pyrazolyl group, a 5-chloro-1,3-dimethyl-4-pyrazolyl group, a pyrazol-1-yl group, a 3-chloro-pyrazol-1-yl group, a 3-bromopyrazol-1-yl group, a 4-chloropyrazol-1-yl group, a 4-bromopyrazol-1-yl group, an imidazole-1-yl group, a 1,2,4-triazole-1-yl group, a 3-chloro-1,2,4-triazole-1-yl group, a 1,2,3,4-tetrazol-1-yl group, a 1,2,3,5-tetrazol-1-yl group, a 2-thienyl group, a 3-thienyl group, a 3-trifluoromethyl-1,2,4-triazol-1-yl group, a 4-trifluoromethylpyrazol-1-yl group, a pyrazinyl group, a 4-pyrimidinyl group, a 5-pyrimidinyl group, a 2-pyridyl group, a 3-pyridyl group, a 4-pyridyl group, a 3-fluoro-2-pyridyl group, a 4-fluoro-2-pyridyl group, a 5-fluoro-2-pyridyl group, a 6-fluoro-2-pyridyl group, a 2-pyrimidinyl group, a 3-chloro-5-trifluoromethylpyridin-2-yl group and a 5-trifluoromethylpyridin-2-yl group.

Examples of the “phenyl group (wherein the phenyl group optionally has a C1 to C3 alkyl group optionally having one or more halogen atoms ozone or more halogen atoms)” in the compound of the present invention include a phenyl group, a 2-fluorophenyl group, a 3-fluorophenyl group, a 4-fluorophenyl group, a 2,3-difluorophenyl group, a 2,4-difluorophenyl group, a 2,5-difluorophenyl group, a 2,6-difluorophenyl group, a 3,4-difluorophenyl group, a 3,5-difluorophenyl group, a 2,3,4,5,6-pentafluorophenyl group, a 2-chlorophenyl group, a 3-chlorophenyl group, a 4-chiorophenyl group, a 2-bromophenyl group, a 3-bromophenyl group, a 4-bromophenyl group, a 2-iodophenyl group, a 3-iodophenyl group, a 4-iodophenyl group, a 2-trifluoromethylphenyl group, a 3-trifluoromethylphenyl group, and a 4-trifluoromethylphenyl group.

Examples of the “5- or 6-membered heterocyclic group (wherein the 5- or 6-membered heterocyclic group optionally has one or more halogen atoms or a C1 to C3 alkyl group optionally having one or more halogen atoms)” in the compound of the present invention include 5- or 6-membered nonaromatic heterocyclic groups such as a pyrrolidin-1-yl group, a 3,3,4,4-tetrafluoropyrrolidin-1-yl group, a tetrahydrofuran-2-yl group, a piperidyl group, a morpholinyl group and a thiomorpholinyl group; and 5- or 6-membered aromatic heterocyclic groups such as a 2-pyrrolyl group, a 2-furyl group, a 3-furyl group, a 5-pyrazolyl group, a 4-pyrazolyl group, a 1-pyrrolyl group, a 1-methyl-2-pyrrolyl group, a 5-bromo-2-furyl group, a 2-methyl-3-furyl group, a 2,5-dimethyl-3-furyl group, a 2,4-dimethyl-3-furyl group, a 5-methyl-2-thienyl group, a 3-methyl-2-thienyl group, a 1-methyl-3-trifluoromethyl-5-pyrazolyl group, a 5-chloro-1,3-dimethyl-4-pyrazolyl group, a pyrazol-1-yl group, a 3-chloro-pyrazol-1-yl group, a 3-bromopyrazol-1-yl group, a 4-chloropyrazol-1-yl group, a 4-bromopyrazol-1-yl group, an imidazol-1-yl group, a 1,2,4-triazol-1-yl group, a 3-chloro-1,2,4-triazol-1-yl group, a 1,2,3,4-tetrazol-1-yl group, a 1,2,3,5-tetrazol-1-yl group, a 2-thienyl group, a 3-thienyl group, a 3-trifluoromethyl-1,2,4-triazol-1-yl group, a 4-trifluoromethylpyrazol-1-yl group, a pyrazinyl group, a 4-pyrimidinyl group, a 5-pyrimidinyl group, a 2-pyridyl group, a 3-pyridyl group, a 4-pyridyl group, a 3-fluoro-2-pyridyl group, a 4-fluoro-2-pyridyl group, a 5-fluoro-2-pyridyl group, a 6-fluoro-2-pyridyl group, a 2-pyrimidinyl group, a 3-chloro-5-trifluoromethylpyridin-2-yl group and a 5-trifluoromethylpyridin-2-yl group.

Examples of the “C1 to C6 alkyl group having one phenyl group (wherein the phenyl group may have one or more atoms or groups selected from group Z)” in the compound of the present invention include a phenylmethyl group, a 4-chlorophenylmethyl group, a 4-trifluoromethylphenylmethyl group, and the like. At that time, when having two or more atoms or groups selected from group Z, the atoms or groups selected from group Z may be the same or different from each other.

Examples of the “C1 to C6 alkyl group having one 5- or 6-membered heterocyclic group (wherein the 5- or 6-membered heterocyclic group may have one or more atoms or groups selected from group Z)” in the compound of the present invention include C1 to C6 alkyl groups having a 5- or 6-membered nonaromatic heterocycle such as a tetrahydrofuran-2-ylmethyl group, a tetrahydropyran-2-ylmethyl group and a tetrahydropyran-3-ylmethyl group; and C1 to C6 alkyl groups having a 5- or 6-membered aromatic heterocycle such as a thiazol-5-ylmethyl group, a 2-chlorothiazol-5-ylmethyl group, a pyridin-3-ylmethyl group, a 6-chloropyridin-3-ylmethyl group and a 6-trifluoromethylpyridin-3-ylmethyl group. At that time, when having two or more atoms or groups selected from group Z, the atoms or groups selected from group Z may be the same or different from each other.

Examples of the “C1 to C6 alkyl group having one thiazolyl group (wherein the thiazolyl group optionally has one or more atoms or substituents selected from the group consisting of C1 to C3 alkyl groups optionally having a halogen atom, C1 to C3 alkoxy groups optionally having a halogen atom, and halogen atoms.)” in the compound of the present invention include a (thiazol-5-yl)methyl group, a (2-chlorothiazol-5-yl)methyl group, and a 1-(2-chlorothiazol-5-yl)ethyl group.

Examples of the “C1 to C6 alkyl group having one pyridyl group (wherein the pyridyl group optionally has one or more atoms or substituents selected from the group consisting of C1 to C3 alkyl groups optionally having a halogen atom, C1 to C3 alkoxy groups optionally having a halogen atom, and halogen atoms.)” in the compound of the present invention include a (pyridin-5-yl)methyl group, a (2-chloropyridin-5-yl)methyl group, a 1-(2-chloropyridin-5-yl)ethyl group, and a (2-trifluoromethylpyridin-5-yl)methyl group.

Examples of the “pyridyl group (wherein the pyridyl group optionally has one or more atoms or substituents selected from the group consisting of C1 to C3 alkyl groups optionally having a halogen atom, C1 to C3 alkoxy groups optionally having a halogen atom, and halogen atoms.)” in the compound of the present invention include a 2-pyridyl group, a 3-pyridyl group, a 4-pyridyl group, a 5-trifluoromethyl-2-pyridyl group, and a 3-chloro-5-trifluoromethyl-2-pyridyl group.

Examples of the “pyrimidinyl group (wherein the pyrimidinyl group optionally has one or more atoms or substituents selected from the group consisting of C1 to C3 alkyl groups optionally having a halogen atom, C1 to C3 alkoxy groups optionally having a halogen atom, and halogen atoms.)” in the compound of the present invention include a 2-pyrimidinyl group, a 4-pyrimidinyl group, a 5-pyrimidinyl group, and a 2-chloro-4-pyrimidinyl group.

Examples of the compound of the present invention include the following compounds.

In the formula (1), compounds wherein R is a C1 to C6 chain hydrocarbon group optionally having one or more atoms or groups selected from group X.

In the formula (1), compounds wherein R¹ is a C3 to C6 cycloalkyl group optionally having one or more atoms or groups selected from group Y.

In the formula (1), compounds wherein R¹ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms, a C2 to C6 alkynyl group optionally having one or more halogen atoms, or C3 to C6 cycloalkyl group optionally having one or more atoms or groups selected from the group consisting of halogen atoms and C1 to C3 alkyl groups.

In the formula (1), compounds wherein R¹ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms, or a C2 to C6 alkynyl group optionally having one or more halogen atoms.

In the formula (1), compounds wherein R¹ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups).

In the formula (1), compounds wherein R¹ is a C3 to C6 cycloalkyl group optionally having one or more atoms or groups selected from the group consisting of halogen atoms and C1 to C3 alkyl groups.

In the formula (1), compounds wherein R¹ is a C2 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups) or a C3 to C6 cycloalkyl group optionally having one or more atoms or groups selected from the group consisting of halogen atoms and C1 to C3 alkyl groups.

In the formula (1), compounds wherein R¹ is a C3 to C6 cycloalkyl group optionally having one or more atoms or groups selected from the group consisting of halogen atoms and C1 to C3 alkyl groups, a C2 to C6 alkyl group optionally having one or more halogen atoms, or a (C3 to C6 cycloalkylalkyl)C1 to C3 alkyl groups optionally having one or more halogen atoms.

In the formula (1), compounds wherein R¹ is a C2 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups).

In the formula (1), compounds wherein R¹ is a C2 to C6 alkyl group, a C2 to C6 alkenyl group, or a C2 to C6 alkynyl group.

In the formula (1), compounds wherein R¹ is a C2 to C6 alkyl group, a cyclopropyl group, or a cyclopropylmethyl group.

In the formula (1), compounds wherein R¹ is a methyl group, an ethyl group, a propyl group, an isopropyl group, a butyl group, a propargyl group, a trifluoromethyl group, a 2,2,2-trifluoroethyl group, a cyclopropyl group, a cyclobutyl group, a cyclopropylmethyl group, or a cyclobutylmethyl group.

In the formula (1), compounds wherein R¹ is an ethyl group, a cyclopropyl group, or a cyclopropylmethyl group.

In the formula (1), compounds wherein R¹ is an ethyl group.

In the formula (1), compounds wherein R is a cyclopropyl group.

In the formula (1), compounds wherein R is a cyclopropylmethyl group.

In the formula (1), compounds wherein R², R³, R⁴ and R⁵ are the same or different and are a C1 to C6 chain hydrocarbon group optionally having one or more atoms or groups selected from group X, a C3 to C6 cycloalkyl group optionally having one or more atoms or groups selected from group Y, or a hydrogen atom.

In the formula (1), compounds wherein R², R³, R⁴ and R⁵ are the same or different and are a phenyl group optionally having one or more atoms or groups selected from group Z, a 5- or 6-membered heterocyclic group optionally having one or more atoms or groups selected from group Z, or a hydrogen atom.

In the formula (1), compounds wherein R², R³, R⁹ and R⁵ are the same or different and are —OR¹², —S(O)ₘR¹², —S(O)₂NR¹²R¹³, —NR¹²R¹³, —NR¹²C(O)R¹³, —NR¹²CO₂R¹³, —NR¹²S(O)₂R¹⁴, —C(O)R¹², —CO₂R¹², —C(O)NR¹²R¹³, —SF₅, a cyano group, a nitro group, a halogen atom, or a hydrogen atom.

In the formula (1), compounds wherein R², R⁴ and R⁵ are the same or different and are a C1 to C6 alkyl group optionally having one or more halogen atoms, —OR¹², —S(O)ₘR¹², a halogen atom or a hydrogen atom, and R³ is a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —NR¹²C(O)R¹³, —NR¹²CO₂R¹³, —NR¹²S(O)₂R¹⁴, —C(O)R¹², —CO₂R¹², —C(O)NR¹²R¹³, —SF₅, a cyano group, a nitro group, a halogen atom, or a hydrogen atom.

In the formula (1), compounds wherein R², R⁴ and R⁵ are the same or different and are a C1 to C6 alkyl group optionally having one or more halogen atoms, —OR¹², —S(O)ₘR¹², a halogen atom or a hydrogen atom, and R³ is a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —C(O)R¹², —CO₂R¹², —C(O)NR¹²R¹³, —SF₅, a cyano group, a nitro group, a halogen atom, or a hydrogen atom.

In the formula (1), compounds wherein R², R⁴ and R⁵ are the same or different and are a C1 to C6 alkyl group optionally having one or more halogen atoms, —OR¹², —S(O)ₘR¹², a halogen atom or a hydrogen atom, and R³ is a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —C(O)R¹², —CO₂R¹², —C(O)NR¹²R¹³, —SF₅, a cyano group, a nitro group or a halogen atom.

In the formula (1), compounds wherein R², R⁴ and R⁵ are the same or different and are a C1 to C6 alkyl group optionally having one or more halogen atoms, —OR¹², —S(O)ₘR¹², a halogen atom or a hydrogen atom, and R³ is a C1 to C6 chain hydrocarbon group optionally having one or more atoms or groups selected from group X, a phenyl group optionally having one or more atoms or groups selected from group Z, a 5- or 6-membered heterocyclic group optionally having one or more atoms or groups selected from group Z, —OR¹², —S(O)ₘR¹², —SF₅, a cyano group, a nitro group, a halogen atom, or a hydrogen atom.

In the formula (1), compounds wherein R², R⁴ and R⁵ are the same or different and are a C1 to C6 alkyl group optionally having one or more halogen atoms, —OR¹², —S(O)ₘR¹², a halogen atom or a hydrogen atom, and R³ is a C1 to C6 chain hydrocarbon group optionally having one or more atoms or groups selected from group X, a phenyl group optionally having one or more atoms or groups selected from group Z, a 5- or 6-membered heterocyclic group optionally having one or more atoms or groups selected from group Z, —OR¹², —S(O)ₘR¹², —SF₅, a cyano group, a nitro group or a halogen atom.

In the formula (1), compounds wherein R², R⁴ and R⁵ are the same or different and are a halogen atom or a hydrogen atom, and R³ is a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —NR¹²R¹³, —NR¹²C(O)R¹³, —NR¹²CO₂R¹³, —NR¹²S(O)₂R¹⁴, —C(O)R¹², —CO₂R¹², —C(O)NR¹²R¹³, —SF₅, a cyano group, a nitro group, a halogen atom, or a hydrogen atom.

In the formula (1), compounds wherein R², R⁴ and R⁵ are the same or different and are a halogen atom or a hydrogen atom, and R³ is a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —C(O)R¹², —CO₂R¹², —C(O)NR¹²R¹³, —SF₅, a cyano group, a nitro group, a halogen atom, or a hydrogen atom.

In the formula (1), compounds wherein R², R⁴ and R⁵ are the same or different and are a halogen atom or a hydrogen atom, and R³ is a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —C(O)R¹², —CO₂R¹², —C(O)NR¹²R¹², —SF₅, a cyano group, a nitro group or a halogen atom.

In the formula (1), compounds wherein R², R⁴ and R⁵ are the same or different and are a halogen atom or a hydrogen atom, and R³ is a C1 to C6 chain hydrocarbon group optionally having one or more atoms or groups selected from group X, a phenyl group optionally having one or more atoms or groups selected from group Z, a 5- or 6-membered heterocyclic group optionally having one or more atoms or groups selected from group Z, —OR¹², —S(O)ₘR¹², —SF₅, a cyano group, a nitro group, a halogen atom, or a hydrogen atom.

In the formula (1), compounds wherein R², R⁴ and R⁵ are the same or different and are a halogen atom, or a hydrogen atom, R³ is a C1 to C6 chain hydrocarbon group optionally having one or more atoms or groups selected from group X, a phenyl group optionally having one or more atoms or groups selected from group Z, a 5- or 6-membered heterocyclic group optionally having one or more atoms or groups selected from group Z, —OR¹², —S(O)ₘR¹², —SF₅, a cyano group, a nitro group or a halogen atom.

In the formula (1), compounds wherein R², R⁴ and R⁵ are the same or different and are a halogen atom or a hydrogen atom, and R³ is a C1 to C6 alkyl group optionally having one or more halogen atoms, a C2 to C6 alkenyl group optionally having one or more halogen atoms, a C2 to C6 alkynyl group optionally having one or more halogen atoms, a 5- or 6-membered heterocyclic group (wherein the 5- or 6-membered heterocyclic group optionally has one or more halogen atoms or a C1 to C3 alkyl group optionally having one or more halogen atoms), —OR¹², —S(O)ₘR¹², a halogen atom or a hydrogen atom.

In the formula (1), compounds wherein R², R⁴ and R⁵ are the same or different and are a halogen atom or a hydrogen atom, and R³ is a C1 to C6 alkyl group optionally having one or more halogen atoms, a C2 to C6 alkenyl group optionally having one or more halogen atoms, a C2 to C6 alkynyl group optionally having one or more halogen atoms, a 5- or 6-membered heterocyclic group (wherein the 5- or 6-membered heterocyclic group optionally has one or more halogen atoms or a C1 to C3 alkyl group optionally having one or more halogen atoms), —OR¹², —S(O)ₘR¹² or a halogen atom.

In the formula (1), compounds wherein R², R⁴ and R⁵ are a hydrogen atom, and R³ is a C1 to C6 alkyl group optionally having one or more halogen atoms, a C2 to C6 alkenyl group optionally having one or more halogen atoms, a C2 to C6 alkynyl group optionally having one or more halogen atoms, a 5- or 6-membered heterocyclic group (wherein the 5- or 6-membered heterocyclic group optionally has one or more halogen atoms or a C1 to C3 alkyl group optionally having one or more halogen atoms), —OR¹², —S(O)ₘR¹², —NR¹²R¹³, —NR¹²C(O)R¹³, —NR¹²CO₂R¹³, —NR¹²S(O)₂R¹⁴, a halogen atom or a hydrogen atom.

In the formula (1), compounds wherein R², R⁴ and R⁵ are a hydrogen atom, and R³ is a C1 to C6 alkyl group optionally having one or more halogen atoms, a C2 to C6 alkenyl group optionally having one or more halogen atoms, a C2 to C6 alkynyl group optionally having one or more halogen atoms, a 5- or 6-membered heterocyclic group (wherein the 5- or 6-membered heterocyclic group optionally has one or more halogen atoms or a C1 to C3 alkyl group optionally having one or more halogen atoms), —OR¹², —S(O)ₘR², a halogen atom or a hydrogen atom.

In the formula (1), compounds wherein R², R⁴ and R⁵ are a hydrogen atom, and R³ is a C1 to C6 alkyl group optionally having one or more halogen atoms, a 5- or 6-membered heterocyclic group (wherein the 5- or 6-membered heterocyclic group optionally has one or more halogen atoms or a C1 to C3 alkyl group optionally having one or more halogen atoms), —OR¹², —S(O)ₘR¹², a halogen atom or a hydrogen atom.

In the formula (1), compounds wherein R², R⁴ and R⁵ are a hydrogen atom, and R³ is a C1 to C6 alkyl group optionally having one or more halogen atoms, a 5- or 6-membered heterocyclic group (wherein the 5- or 6-membered heterocyclic group optionally has one or more halogen atoms or a C1 to C3 alkyl group optionally having one or more halogen atoms), —OR¹², —S(O)ₘR¹², a halogen atom or a hydrogen atom.

In the formula (1), compounds wherein R², R⁴ and R⁵ are a hydrogen atom, and R³ is a C1 to C3 alkyl group optionally having one or more halogen atoms, a C1 to C3 alkoxy group optionally having one or more halogen atoms, a C1 to C3 alkylsulfanyl group optionally having one or more halogen atoms, a C1 to C3 alkylsulfinyl group optionally having one or more halogen atoms, a C1 to C3 alkylsulfonyl group optionally having one or more halogen atoms, a pyridyl group (wherein the pyridyl group optionally has one or more atoms or substituents selected from the group consisting of halogen atoms, C1 to C3 alkyl groups optionally having a halogen atom, and C1 to C3 alkoxy groups optionally having a halogen atom.), a pyrimidinyl group (wherein the pyrimidinyl group optionally has one or more atoms or substituents selected from the group consisting of halogen atoms, C1 to C3 alkyl groups optionally having a halogen atom, and C1 to C3 alkoxy groups optionally having a halogen atom.) or a halogen atom.

In the formula (1), compounds wherein R², R⁴ and R⁵ are a hydrogen atom, and R³ is a C1 to C3 alkyl group optionally having one or more halogen atoms.

In the formula (1), compounds wherein R², R⁴ and R⁵ are a hydrogen atom, and R³ is a C1 to C3 alkoxy group optionally having one or more halogen atoms.

In the formula (1), compounds wherein R², R⁴ and R⁵ are a hydrogen atom, and R³ is a C1 to C3 alkylsulfanyl group optionally having one or more halogen atoms, a C1 to C3 alkylsulfinyl group optionally having one or more halogen atoms, or a C1 to C3 alkylsulfonyl group optionally having one or more halogen atoms.

In the formula (1), compounds wherein R², R⁴ and R⁵ are a hydrogen atom, and R³ is a C1 to C6 alkylamino group optionally having one or more halogen atoms.

In the formula (1), compounds wherein R², R⁴ and R⁵ are a hydrogen atom, and R³ is a C2 to C8 dialkylamino group optionally having one or more halogen atoms.

In the formula (1), compounds wherein R², R⁴ and R⁵ are a hydrogen atom, and R³ is a C2 to C6 alkylcarbonylamino group optionally having one or more halogen atoms.

In the formula (1), compounds wherein R², R⁴ and R⁵ are a hydrogen atom, and R³ is a C2 to C6 alkoxycarbonylamino group optionally having one or more halogen atoms.

In the formula (1), compounds wherein R², R⁴ and R⁵ are a hydrogen atom, and R³ is a C2 to C6 alkylcarbonyl group optionally having one or more halogen atoms.

In the formula (1), compounds wherein R², R⁴ and R⁵ are a hydrogen atom, and R³ is a C2 to C6 alkoxycarbonyl group optionally having one or more halogen atoms.

In the formula (1), compounds wherein R², R⁴ and R⁵ are a hydrogen atom, and R³ is a C1 to C3 alkylsulfonylamino group optionally having one or more halogen atoms.

In the formula (1), compounds wherein R², R⁴ and R⁵ are a hydrogen atom, and R³ is an amino group.

In the formula (1), compounds wherein R², R⁴ and R⁵ are a hydrogen atom, and R³ is a C2 to C6 alkylaminocarbonyl group optionally having one or more halogen atoms.

In the formula (1), compounds wherein R², R⁴ and R⁵ are a hydrogen atom, and R³ is a C3 to C8 dialkylaminocarbonyl group optionally having one or more halogen atoms.

In the formula (1), compounds wherein R², R⁴ and R⁵ are a hydrogen atom, and R³ is an aminocarbonyl group.

In the formula (1), compounds wherein R², R⁴ and R⁵ are a hydrogen atom, and R³ is a pyridyl group (wherein the pyridyl group optionally has one or more atoms or substituents selected from the group consisting of halogen atoms, C1 to C3 alkyl groups optionally having a halogen atom, and C1 to C3 alkoxy groups optionally having a halogen atom.) or a pyrimidinyl group (wherein the pyrimidinyl group optionally has one or more atoms or substituents selected from the group consisting of halogen atoms, C1 to C3 alkyl groups optionally having a halogen atom, and C1 to C3 alkoxy groups optionally having a halogen atom.).

In the formula (1), compounds wherein R², R⁴ and R⁵ are a hydrogen atom, and R³ is a halogen atom.

In the formula (1), compounds wherein R², R⁴ and R⁵ are a hydrogen atom, and R³ is a methyl group, an ethyl group, a vinyl group, a propyl group, an isopropyl group, a cyclopropyl group, a propargyl group, a trifluoromethyl group, a pentafluoroethyl group, a heptafluoropropyl group, a heptafluoroisopropyl group, a methoxy group, a trifluoromethoxy group, a methylsulfanyl group, a trifluoromethylsulfanyl group, a methylsulfinyl group, a trifluoromethylsulfinyl group, a methylsulfonyl group, a trifluoromethylsulfonyl group, a 2-pyridyl group, a 2-pyrimidinyl group, a 5-trifluoromethyl-2-pyridyl group, a 3-chloro-5-trifluoromethyl-2-pyridyl group, a fluorine atom, a chlorine atom, a bromine atom, or an iodine atom.

In the formula (1), compounds wherein R², R⁴ and R⁵ are a hydrogen atom, and R³ is a methyl group, an ethyl group, a vinyl group, a propyl group, an isopropyl group, a cyclopropyl group, a propargyl group, a trifluoromethyl group, a pentafluoroethyl group, a heptafluoropropyl group, a heptafluoroisopropyl group, a methoxy group, a trifluoromethoxy group, a methylsulfanyl group, a trifluoromethylsulfanyl group, a methylsulfinyl group, a trifluoromethylsulfinyl group, a methylsulfonyl group, a trifluoromethylsulfonyl group, an acetylamino group, a methoxycarbonylamino group, an ethoxycarbonylamino group, a tert-butyloxycarbonyl amino group, a methylsulfonylamino group, an amino group, an acetyl group, a methoxycarbonyl group, an ethoxycarbonyl group, a methylaminocarbonyl group, an ethylaminocarbonyl group, a dimethylaminocarbonyl group, a diethylaminocarbonyl group, an aminocarbonyl group, a fluorine atom, a chlorine atom, a bromine atom, or an iodine atom.

In the formula (1), compounds wherein R², R⁴ and R⁵ are a hydrogen atom, and R³ is a methyl group, an ethyl group, a vinyl group, a propyl group, an isopropyl group, a cyclopropyl group, a propargyl group, a trifluoromethyl group, a pentafluoroethyl group, a heptafluoropropyl group, a heptafluoroisopropyl group, a methoxy group, a trifluoromethoxy group, a methylsulfanyl group, a trifluoromethylsulfanyl group, a methylsulfinyl group, a trifluoromethylsulfinyl group, a methylsulfonyl group, a trifluoromethylsulfonyl group, a fluorine atom, a chlorine atom, a bromine atom, or an iodine atom.

In the formula (1), compounds wherein R², R³, R⁴ and R⁵ are a hydrogen atom.

In the formula (1), compounds wherein R², R⁴ and R⁵ are the same or different and are a C1 to C6 alkyl group optionally having one or more halogen atoms, —OR¹², —S(O)ₘR¹², a halogen atom, or a hydrogen atom.

In the formula (1), compounds wherein R², R⁴ and R⁵ are the same or different and are a C1 to C6 alkyl group optionally having one or more halogen atoms or a hydrogen atom.

In the formula (1), compounds wherein R², R⁴ and R⁵ are the same or different and are —OR¹², —S(O)ₘR¹², a halogen atom, or a hydrogen atom.

In the formula (1), compounds wherein R², R⁴ and R⁵ are the same or different and are a halogen atom or a hydrogen atom.

In the formula (1), compounds wherein R², R⁴ and R⁵ are a hydrogen atom.

In the formula (1), compounds wherein R³ is a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —C(O)R¹², —CO₂R¹², —C(O)NR¹²R¹³, —SF₅, a cyano group, a nitro group, a halogen atom, or a hydrogen atom.

In the formula (1), compounds wherein R³ is a C1 to C6 alkyl group optionally having one or more halogen atoms, a C2 to C6 alkenyl group optionally having one or more halogen atoms, a C2 to C6 alkynyl group optionally having one or more halogen atoms, a 5- or 6-membered heterocyclic group (wherein the 5- or 6-membered heterocyclic group optionally has one or more halogen atoms or a C1 to C3 alkyl group optionally having one or more halogen atoms), —OR¹², —S(O)ₘR¹², a halogen atom, or a hydrogen atom.

In the formula (1), compounds wherein R³ is a C1 to C3 alkyl group optionally having one or more halogen atoms, a C1 to C3 alkoxy group optionally having one or more halogen atoms, a C1 to C3 alkylsulfanyl group optionally having one or more halogen atoms, a C1 to C3 alkylsulfinyl group optionally having one or more halogen atoms, a C1 to C3 alkylsulfonyl group optionally having one or more halogen atoms, a pyridyl group (wherein the pyridyl group optionally has one or more atoms or substituents selected from the group consisting of halogen atoms, C1 to C3 alkyl groups optionally having a halogen atom, and C1 to C3 alkoxy groups optionally having a halogen atom.), a pyrimidinyl group (wherein the pyrimidinyl group optionally has one or more atoms or substituents selected from the group consisting of halogen atoms, C1 to C3 alkyl groups optionally having a halogen atom, and C1 to C3 alkoxy groups optionally having a halogen atom.), a halogen atom, or a hydrogen atoms.

In the formula (1), compounds wherein R³ is a C1 to C3 alkyl group optionally having one or more halogen atoms, a C1 to C3 alkoxy group optionally having one or more halogen atoms, a C1 to C3 alkylsulfanyl group optionally having one or more halogen atoms, a C1 to C3 alkylsulfinyl group optionally having one or more halogen atoms, a C1 to C3 alkylsulfonyl group optionally having one or more halogen atoms, a pyridyl group (wherein the pyridyl group optionally has one or more atoms or substituents selected from the group consisting of halogen atoms, C1 to C3 alkyl groups optionally having a halogen atom, and C1 to C3 alkoxy groups optionally having a halogen atom.), a pyrimidinyl group (wherein the pyrimidinyl group optionally has one or more atoms or substituents selected from the group consisting of halogen atoms, C1 to C3 alkyl groups optionally having a halogen atom, and C1 to C3 alkoxy groups optionally having a halogen atom.), or a halogen atom.

In the formula (1), compounds wherein R³ is a C1 to C3 alkyl group optionally having one or more halogen atoms, a C1 to C3 alkoxy group optionally having one or more halogen atoms, a C1 to C3 alkylsulfanyl group optionally having one or more halogen atoms, a C1 to C3 alkylsulfinyl group optionally having one or more halogen atoms, a C1 to C3 alkylsulfonyl group optionally having one or more halogen atoms, a pyridyl group (wherein the pyridyl group optionally has one or more atoms or substituents selected from the group consisting of halogen atoms, C1 to C3 alkyl groups optionally having a halogen atom, and C1 to C3 alkoxy groups optionally having a halogen atom.), a pyrimidinyl group (wherein the pyrimidinyl group optionally has one or more atoms or substituents selected from the group consisting of halogen atoms, C1 to C3 alkyl groups optionally having a halogen atom, and C1 to C3 alkoxy groups optionally having a halogen atom.), a halogen atom, or a hydrogen atoms.

In the formula (1), compounds wherein R³ is a C1 to C3 alkyl group optionally having one or more halogen atoms, a C1 to C3 alkoxy group optionally having one or more halogen atoms, a C1 to C3 alkylsulfanyl group optionally having one or more halogen atoms, a C1 to C3 alkylsulfinyl group optionally having one or more halogen atoms, a C1 to C3 alkylsulfonyl group optionally having one or more halogen atoms, a C1 to C6 alkylamino group optionally having one or more halogen atoms, a C2 to C8 dialkylamino group optionally having one or more halogen atoms, a C2 to C6 alkylcarbonylamino group optionally having one or more halogen atoms, a C2 to C6 alkoxycarbonylamino group optionally having one or more halogen atoms, a C1 to C3 alkylsulfonylamino group optionally having one or more halogen atoms, an amino group, a C2 to C6 alkylcarbonyl group optionally having one or more halogen atoms, a C2 to C6 alkoxycarbonyl group optionally having one or more halogen atoms, a C2 to C6 alkylaminocarbonyl group optionally having one or more halogen atoms, a C3 to C8 dialkylaminocarbonyl group optionally having one or more halogen atoms, an aminocarbonyl group, a pyridyl group (wherein the pyridyl group optionally has one or more atoms or substituents selected from the group consisting of halogen atoms, C1 to C3 alkyl groups optionally having a halogen atom, and C1 to C3 alkoxy groups optionally having a halogen atom.), a pyrimidinyl group (wherein the pyrimidinyl group optionally has one or more atoms or substituents selected from the group consisting of halogen atoms, C1 to C3 alkyl groups optionally having a halogen atom, and C1 to C3 alkoxy groups optionally having a halogen atom.), a halogen atom, or a hydrogen atom.

In the formula (1), compounds wherein R³ is a methyl group, an ethyl group, a vinyl group, a propyl group, an isopropyl group, a cyclopropyl group, a propargyl group, a trifluoromethyl group, a pentafluoroethyl group, a heptafluoropropyl group, a heptafluoroisopropyl group, a methoxy group, a trifluoromethoxy group, a methylsulfanyl group, a trifluoromethylsulfanyl group, a methylsulfinyl group, a trifluoromethylsulfinyl group, a methylsulfonyl group, a trifluoromethylsulfonyl group, a 2-pyridyl group, a 2-pyrimidinyl group, a 5-trifluoromethyl-2-pyridyl group, a 3-chloro-5-trifluoromethyl-2-pyridyl group, a fluorine atom, a chlorine atom, a bromine atom, or an iodine atom.

In the formula (1), compounds wherein R³ is a methyl group, an ethyl group, a vinyl group, a propyl group, an isopropyl group, a cyclopropyl group, a propargyl group, a trifluoromethyl group, a pentafluoroethyl group, a heptafluoropropyl group, a heptafluoroisopropyl group, a methoxy group, a trifluoromethoxy group, a methylsulfanyl group, a trifluoromethylsulfanyl group, a methylsulfinyl group, a trifluoromethylsulfinyl group, a methylsulfonyl group, a trifluoromethylsulfonyl group, an acetylamino group, a methoxycarbonylamino group, an ethoxycarbonylamino group, a tert-butoxycarbonylamino group, a methylsulfonylamino group, an amino group, an acetyl group, a methoxycarbonyl group, an ethoxycarbonyl group, a methylaminocarbonyl group, an ethylaminocarbonyl group, a dimethylaminocarbonyl group, a diethylaminocarbonyl group, an aminocarbonyl group, a fluorine atom, a chlorine atom, a bromine atom, or an iodine atom.

In the formula (1), compounds wherein R³ is a methyl group, an ethyl group, a vinyl group, a propyl group, an isopropyl group, a cyclopropyl group, a propargyl group, a trifluoromethyl group, a pentafluoroethyl group, a heptafluoropropyl group, a heptafluoroisopropyl group, a methoxy group, a trifluoromethoxy group, a methylsulfanyl group, a trifluoromethylsulfanyl group, a methylsulfinyl group, a trifluoromethylsulfinyl group, a methylsulfonyl group, a trifluoromethylsulfonyl group, a fluorine atom, a chlorine atom, a bromine atom, or an iodine atom.

In the formula (1), compounds wherein R⁶ is a C1 to C6 chain hydrocarbon group optionally having one or more atoms or groups selected from group W or a C3 to C6 cycloalkyl group optionally having one or more atoms or groups selected from group Y.

In the formula (1), compounds wherein R⁶ is a C1 to C6 alkyl group having one phenyl group (wherein the phenyl group optionally has one or more atoms or groups selected from group Z) or a C1 to C6 alkyl group having one 5- or 6-membered heterocyclic group (wherein the 5- or 6-membered heterocyclic group optionally has one or more atoms or groups selected from group Z).

In the formula (1), compounds wherein R⁶ is a —C(O)R¹², —C(O)OR¹² or —C(O)NR¹²R¹³.

In the formula (1), compounds wherein R⁶ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms, a C2 to C6 alkynyl group optionally having one or more halogen atoms, a C1 to C6 alkyl group having one thiazolyl group (wherein the thiazolyl group optionally has one or more atoms or substituents selected from the group consisting of halogen atoms, C1 to C3 alkyl groups optionally having a halogen atom, and C1 to C3 alkoxy groups optionally having a halogen atom.), a C1 to C6 alkyl group having one pyridyl group (wherein the pyridyl group optionally has one or more atoms or substituents selected from the group consisting of halogen atoms, C1 to C3 alkyl groups optionally having a halogen atom, and C1 to C3 alkoxy groups optionally having a halogen atom.), a (C1 to C6 alkoxy)C1 to C6 alkyl group optionally having one or more halogen atoms, —C(O)R¹², or —C(O)OR¹².

In the formula (1), compounds wherein R⁶ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms, or a C2 to C6 alkynyl group optionally having one or more halogen atoms.

In the formula (1), compounds wherein R⁶ is a C1 to C6 alkyl group having one thiazolyl group (wherein the thiazolyl group optionally has one or more atoms or substituents selected from the group consisting of halogen atoms, C1 to C3 alkyl groups optionally having a halogen atom, and C1 to C3 alkoxy groups optionally having a halogen atom.) or a C1 to C6 alkyl group having one pyridyl group (wherein the pyridyl group optionally has one or more atoms or substituents selected from the group consisting of halogen atoms, C1 to C3 alkyl groups optionally having a halogen atom, and C1 to C3 alkoxy groups optionally having a halogen atom.).

In the formula (1), compounds wherein R⁶ is a (C1 to C6 alkoxy)C1 to C6 alkyl group optionally having one or more halogen atoms, —C(O)R¹², or —C(O)OR¹².

In the formula (1), compounds wherein R⁶ is a C1 to C6 alkyl group optionally having one or more halogen atoms, a C2 to C6 alkenyl group optionally having one or more halogen atoms, or a C2 to C6 alkynyl group optionally having one or more halogen atoms.

In the formula (1), compounds wherein R⁶ is a C1 to C6 alkyl group, a C2 to C6 alkenyl group, or a C2 to C6 alkynyl group, a (C1 to C6 alkoxy)C1 to C6 alkyl group, —C(O)R¹², or —C(O)OR¹².

In the formula (1), compounds wherein R⁶ is a C1 to C6 alkyl group, a C2 to C6 alkenyl group, or a C2 to C6 alkynyl group.

In the formula (1), compounds wherein R⁶ is a C1 to C6 alkyl group optionally having one or more atoms or groups selected from the group consisting of halogen atoms and a cyclopropyl group or a propargyl group.

In the formula (1), compounds wherein R⁶ is a C1 to C6 alkyl group optionally having one or more atoms or groups selected from the group consisting of halogen atoms and a cyclopropyl group.

In the formula (1), compounds wherein R⁶ is a methyl group, an ethyl group, a propyl group, an isopropyl group, a butyl group, an isobutyl group, a hexyl group, a propargyl group, a cyclopropyl group, a cyclobutyl group, a cyclohexyl group, a cyclopropylmethyl group, a cyclobutylmethyl group, a 2,2,2-trifluoroethyl group, a benzyl group, a 6-chloropyridin-3-ylmethyl group, a 2-chlorothiazolyl-5-ylmethyl group, a methoxymethyl group, an ethoxymethyl group, a methoxycarbonyl group, or an ethoxycarbonyl group.

In the formula (1), compounds wherein R⁶ is a methyl group, an ethyl group, a propyl group, an isopropyl group, a butyl group, an isobutyl group, a hexyl group, a propargyl group, a cyclopropyl group, a cyclobutyl group, a cyclohexyl group, a cyclopropylmethyl group, a cyclobutylmethyl group, a 2,2,2-trifluoroethyl group, a benzyl group, a 6-chloropyridin-3-ylmethyl group, or a 2-chlorothiazolyl-5-ylmethyl group.

In the formula (1), compounds wherein R⁶ is a methyl group, an ethyl group, a propyl group, an isopropyl group, a butyl group, an isobutyl group, or a hexyl group.

In the formula (1), compounds wherein R⁶ is a cyclopropyl group, a cyclobutyl group, a cyclohexyl group, a cyclopropylmethyl group, or a cyclobutylmethyl group.

In the formula (1), compounds wherein R⁶ is a benzyl group, a 6-chloropyridin-3-ylmethyl group, or a 2-chlorothiazolyl-5-ylmethyl group.

In the formula (1), compounds wherein R⁶ is a methoxymethyl group, an ethoxymethyl group, a methoxycarbonyl group, or an ethoxycarbonyl group.

In the formula (1), compounds wherein R⁶ is a methyl group.

In the formula (1), compounds wherein R⁶ is an ethyl group.

In the formula (1), compounds wherein R⁶ is a cyclopropylmethyl group.

In the formula (1), compounds wherein R⁷ and R¹¹ are the same or different and are a C1 to C6 chain hydrocarbon group optionally having one or more atoms or groups selected from group X or a hydrogen atom.

In the formula (1), compounds wherein R⁷ and R¹¹ are the same or different and are a C1 to C6 chain hydrocarbon group optionally having one or more atoms or groups selected from group X, —OR¹⁶, —S(O)ₘR¹⁶, a fluorine atom, or a hydrogen atom.

In the formula (1), compounds wherein R⁷ and R¹¹ are the same or different and are a C1 to C6 alkyl group optionally having one or more halogen atoms, —OR¹⁶, —S(O)ₘR¹⁶, a fluorine atom, or a hydrogen atom.

In the formula (1), compounds wherein R⁷ and R¹¹ are the same or different and are a fluorine atom or a hydrogen atom.

In the formula (1), compounds wherein R⁷ and R¹¹ are a hydrogen atom.

In the formula (1), compounds wherein R⁸, R⁹ and R¹⁰ are the same or different and are a C1 to C6 chain hydrocarbon group optionally having one or more atoms or groups selected from group X, —OR¹², —S(O)ₘR¹², —SF₅, a halogen atom, or a hydrogen atom.

In the formula (1), compounds wherein R⁸, R⁹ and R¹⁰ are the same or different and are a phenyl group optionally having one or more atoms or groups selected from group Z, a 5- or 6-membered heterocyclic group optionally having one or more atoms or groups selected from group Z, or a hydrogen atom.

In the formula (1), compounds wherein R⁸, R⁹ and R¹⁰ are the same or different and are a C1 to C6 chain hydrocarbon group optionally having one or more atoms or groups selected from group X, a halogen atom, or a hydrogen atom.

In the formula (1), compounds wherein R⁸, R⁹ and R¹⁰ are the same or different and are —OR¹², —S(O)ₘR¹², a halogen atom, or a hydrogen atom.

In the formula (1), compounds wherein R⁸, R⁹ and R¹⁰ are the same or different and are a C1 to C6 alkyl group optionally having one or more halogen atoms, a C2 to C6 alkenyl group optionally having one or more halogen atoms, a C2 to C6 alkynyl group optionally having one or more halogen atoms, —OR¹², —S(O)ₘR¹², a halogen atom, or a hydrogen atom.

In the formula (1), compounds wherein R⁸, R⁹ and R¹⁰ are the same or different and are a C1 to C6 haloalkyl group, a C2 to C6 haloalkenyl group, a C1 to C6 haloalkoxy group, a C1 to C6 haloalkylsulfanyl group, a C1 to C6 haloalkylsulfinyl group, a C1 to C6 haloalkylsulfonyl group, a halogen atom, or a hydrogen atom.

In the formula (1), compounds wherein R⁸, R⁹ and R¹⁰ are the same or different and are a C1 to C2 haloalkyl group, a C2 to C6 haloalkenyl group, a C1 to C6 haloalkoxy group, a C1 to C6 haloalkylsulfanyl group, a C1 to C6 haloalkylsulfinyl group, a C1 to C6 haloalkylsulfonyl group, a halogen atom, or a hydrogen atom.

In the formula (1), compounds wherein R⁸, R⁹ and R¹⁰ are the same or different and are a C1 to C2 haloalkyl group, a C2 to C6 haloalkenyl group, a C1 to C6 haloalkylsulfanyl group, a C1 to C6 haloalkylsulfinyl group, a C1 to C6 haloalkylsulfonyl group, a halogen atom, or a hydrogen atom.

In the formula (1), compounds wherein R⁸, R⁹ and R¹⁰ are the same or different and are a C1 to C3 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.

In the formula (1), compounds wherein R⁸, R⁹ and R¹⁰ are the same or different and are a C1 to C2 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.

In the formula (1), compounds wherein R⁸, R⁹ and R¹⁰ are the same or different and are a C1 to C2 perfluoroalkyl group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.

In the formula (1), compounds wherein R⁸, R⁹ and R¹⁰ are the same or different and are a trifluoromethyl group, a pentafluoroethyl group, a heptafluoropropyl group, a heptafluoroisopropyl group, a trifluoromethoxy group, a trifluoromethylsulfanyl group, a trifluoromethylsulfinyl group or a trifluoromethylsulfonyl group, a fluorine atom, a chlorine atom, a bromine atom, or a hydrogen atom.

In the formula (1), compounds wherein R⁸, R⁹ and R¹⁰ are the same or different and are a trifluoromethyl group, a pentafluoroethyl group, a trifluoromethoxy group, a trifluoromethylsulfanyl group, a trifluoromethylsulfinyl group or a trifluoromethylsulfonyl group, a fluorine atom, a chlorine atom, a bromine atom, or a hydrogen atom.

In the formula (1), compounds wherein R⁸, R⁹ and R¹⁰ are the same or different and are a trifluoromethyl group, a pentafluoroethyl group, a trifluoromethylsulfanyl group, a trifluoromethylsulfinyl group or a trifluoromethylsulfonyl group, a fluorine atom, a chlorine atom, a bromine atom, or a hydrogen atom.

In the formula (1), compounds wherein R⁷ and R¹¹ are the same or different and are a C1 to C6 alkyl group optionally having one or more halogen atoms, —OR¹⁶, —S(O)ₘR¹⁶, a fluorine atom, or a hydrogen atom, and R⁸, R⁹ and R¹⁰ are the same or different and are a C1 to C6 chain hydrocarbon group optionally having one or more atoms or groups selected from group X, —OR¹², —S(O)ₘR¹², —SF₅, a halogen atom, or a hydrogen atom.

In the formula (1), compounds wherein R⁷ and R¹¹ are the same or different and are a fluorine atom, or a hydrogen atom, and R⁸, R⁹ and R¹⁰ are the same or different and are a C1 to C6 alkyl group optionally having one or more halogen atoms, a C2 to C6 alkenyl group optionally having one or more halogen atoms, a C2 to C6 alkynyl group optionally having one or more halogen atoms, —OR¹², —S(O)ₘR¹², a halogen atom, or a hydrogen atom.

In the formula (1), compounds wherein R⁷ and R¹¹ are a hydrogen atom, and R⁸, R⁹ and R¹⁰ are the same or different and are a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —S(O)₂NR¹²R¹³, —NR¹²R¹³, —NR¹²C(O) R¹³, —NR¹²CO₂R¹³, —NR¹²S(O)₂R¹⁴, —C(O)R¹², —CO₂R¹⁴, —C(O)NR¹⁵R¹⁶, —SF₅, a cyano group, a nitro group, a halogen atom, or a hydrogen atom.

In the formula (1), compounds wherein R⁷ and R¹¹ are a hydrogen atom, and R⁸, R⁹ and R¹⁰ are the same or different and are a C1 to C6 chain hydrocarbon group optionally having one or more atoms or groups selected from group X, —OR¹², —S(O)ₘR¹², —SF₅, a halogen atom, or a hydrogen atom.

In the formula (1), compounds wherein R⁷ and R¹¹ are a hydrogen atom, and R⁸, R⁹ and R¹⁰ are the same or different and are a C1 to C6 haloalkyl group, a C2 to C6 haloalkenyl group, a C1 to C6 haloalkoxy group, a C1 to C6 haloalkylsulfanyl group, a C1 to C6 haloalkylsulfinyl group, a C1 to C6 haloalkylsulfonyl group, a halogen atom, or a hydrogen atom.

In the formula (1), compounds wherein R⁷ and R¹¹ are a hydrogen atom, and R⁸, R⁹ and R¹⁰ are the same or different and are a C1 to C2 haloalkyl group, a C2 to C6 haloalkenyl group, a C1 to C6 haloalkoxy group, a C1 to C6 haloalkylsulfanyl group, a C1 to C6 haloalkylsulfinyl group, a C1 to C6 haloalkylsulfonyl group, a halogen atom, or a hydrogen atom.

In the formula (1), compounds wherein R⁷ and R¹¹ are a hydrogen atom, and R⁸, R⁹ and R¹⁰ are the same or different and are a C1 to C2 haloalkyl group, a C2 to C6 haloalkenyl group, a C1 to C6 haloalkylsulfanyl group, a C1 to C6 haloalkylsulfinyl group, a C1 to C6 haloalkylsulfonyl group, a halogen atom, or a hydrogen atom.

In the formula (1), compounds wherein R⁷ and R¹¹ are a hydrogen atom, and R⁸, R⁹ and R¹⁰ are the same or different and are a C1 to C3 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.

In the formula (1), compounds wherein R⁷ and R¹¹ are a hydrogen atom, and R⁸, R⁹ and R¹⁰ are the same or different and are a C1 to C2 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.

In the formula (1), compounds wherein R⁷ and R¹¹ are a hydrogen atom, and R⁸, R⁹ and R¹⁰ are the same or different and are a C1 to C2 perfluoroalkyl group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.

In the formula (1), compounds wherein R⁷ and R¹¹ are a hydrogen atom, and R⁸, R⁹ and R¹⁰ are the same or different and are a trifluoromethyl group, a pentafluoroethyl group, a heptafluoropropyl group, a heptafluoroisopropyl group, a trifluoromethoxy group, a trifluoromethylsulfanyl group, a trifluoromethylsulfinyl group or a trifluoromethylsulfonyl group, a fluorine atom, a chlorine atom, a bromine atom, or a hydrogen atom.

In the formula (1), compounds wherein R⁷ and R¹¹ are a hydrogen atom, and R⁸, R⁹ and R¹⁰ are the same or different and are a trifluoromethyl group, a pentafluoroethyl group, a trifluoromethoxy group, a trifluoromethylsulfanyl group, a trifluoromethylsulfinyl group or a trifluoromethylsulfonyl group, a fluorine atom, a chlorine atom, a bromine atom, or a hydrogen atom.

In the formula (1), compounds wherein R⁷ and R¹¹ are a hydrogen atom, and R⁸, R⁹ and R¹⁰ are the same or different and are a trifluoromethyl group, a pentafluoroethyl group, a trifluoromethylsulfanyl group, a trifluoromethylsulfinyl group or a trifluoromethylsulfonyl group, a fluorine atom, a chlorine atom, a bromine atom, or a hydrogen atom.

In the formula (1), compounds wherein R⁷ and R¹¹ are a hydrogen atom, and R⁸, R⁹ and R¹⁰ are the same or different and are a pentafluoroethyl group, a heptafluoroisopropyl group, a trifluoromethoxy group, a trifluoromethylsulfanyl group, a trifluoromethylsulfinyl group or a trifluoromethylsulfonyl group, or a hydrogen atom.

In the formula (1), compounds wherein R⁷ and R¹¹ are a hydrogen atom, and R⁸, R⁹ and R¹⁰ are the same or different and are a pentafluoroethyl group, a trifluoromethoxy group, a trifluoromethylsulfanyl group, a trifluoromethylsulfinyl group or a trifluoromethylsulfonyl group, or a hydrogen atom.

In the formula (1), compounds wherein R⁷ and R¹¹ are a hydrogen atom, and R⁸, R⁹ and R¹⁰ are the same or different and are a pentafluoroethyl group, a trifluoromethylsulfanyl group, a trifluoromethylsulfinyl group or a trifluoromethylsulfonyl group, or a hydrogen atom.

In the formula (1), compounds wherein R⁷, R⁸, R¹⁰ and R¹¹ are a hydrogen atom.

In the formula (1), compounds wherein R⁷, R⁹ and R¹¹ are a hydrogen atom.

In the formula (1), compounds wherein R⁷, R⁸, R¹⁰ and R¹¹ are a hydrogen atom, and R⁹ is a C1 to C3 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group or a halogen atom.

In the formula (1), compounds wherein R⁷, R⁹ and R¹¹ are a hydrogen atom, and R⁸ and R¹⁰ are the same or different and are a C1 to C3 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.

In the formula (1), compounds wherein Q is an oxygen atom.

In the formula (1), compounds wherein Q is a sulfur atom.

In the formula (1), compounds wherein m is 0.

In the formula (1), compounds wherein m is 1.

In the formula (1), compounds wherein m is 2.

In the formula (1), compounds wherein n is 0.

In the formula (1), compounds wherein n is 1.

In the formula (1), compounds wherein n is 2.

In the formula (1), compounds, wherein

R¹ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms, a C2 to C6 alkynyl group optionally having one or more halogen atoms, or a C3 to C6 cycloalkyl group optionally having one or more atoms or groups selected from the group consisting of halogen atoms and C1 to C3 alkyl groups,

R², R⁴ and R⁵ are the same or different and are a C1 to C6 alkyl group optionally having one or more halogen atoms, —S(O)ₘR¹², a halogen atom or a hydrogen atom,

R³ is a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —C(O)R¹², —CO₂R¹², —C(O)NR¹²R¹³, —SF₅, a cyano group, a nitro group, a halogen atom, or a hydrogen atom,

R⁶ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms, a C2 to C6 alkynyl group optionally having one or more halogen atoms, a C1 to C6 alkyl group having one thiazolyl group (wherein the thiazolyl group optionally has one or more atoms or substituents selected from the group consisting of halogen atoms, C1 to C3 alkyl groups optionally having a halogen atom, and C1 to C3 alkoxy groups optionally having a halogen atom.), a C1 to C6 alkyl group having one pyridyl group (wherein the pyridyl group optionally has one or more atoms or substituents selected from the group consisting of halogen atoms, C1 to C3 alkyl groups optionally having a halogen atom, and C1 to C3 alkoxy groups optionally having a halogen atom.), a (C1 to C6 alkoxy) C1 to C6 alkyl group optionally having one or more halogen atoms, —C(O)R¹² or —C(O)OR¹²,

R⁷ and R¹¹ are the same or different and are a C1 to C6 alkyl group optionally having one or more halogen atoms, —OR¹⁶, —S(O)ₘR¹⁶, a fluorine atom or a hydrogen atom,

R⁸, R⁹ and R¹⁰ are the same or different and are a C1 to C6 chain hydrocarbon group optionally having one or more atoms or groups selected from group X, —OR¹², —S(O)ₘR¹², —SF₅, a halogen atom or a hydrogen atom,

R¹² and R¹³ are the same or different and are a C1 to C6 alkyl group optionally having one or more halogen atoms, a phenyl group (wherein the phenyl group optionally has a C1 to C3 alkyl group optionally having one or more halogen atoms or one or more halogen atoms), a 5- or 6-membered heterocyclic group (wherein the 5- or 6-membered heterocyclic group optionally has a C1 to C3 alkyl group optionally having one or more halogen atoms or one or more halogen atoms) or a hydrogen atom, and

Q is an oxygen atom.

In the formula (1), compounds according to [1], wherein

R¹ is a C3 to C6 cycloalkyl group optionally having one or more atoms or groups selected from the group consisting of halogen atoms and C1 to C3 alkyl groups, a C2 to C6 alkyl group optionally having one or more halogen atoms or a (C3 to C6 cycloalkylalkyl)C1 to C3 alkyl group optionally having one or more halogen atoms,

R², R⁴ and R⁵ are the same or different and are a halogen atom or a hydrogen atom,

R³ is a C1 to C6 alkyl group optionally having one or more halogen atoms, a C2 to C6 alkenyl group optionally having one or more halogen atoms, a C2 to C6 alkynyl group optionally having one or more halogen atoms, a 5- or 6-membered heterocyclic group (wherein the 5- or 6-membered heterocyclic group optionally has a C1 to C3 alkyl group optionally having one or more halogen atoms or one or more halogen atoms), —OR¹², —S(O)ₘR¹², a halogen atom, or a hydrogen atom,

R⁶ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms or a C2 to C6 alkynyl group optionally having one or more halogen atoms,

R⁷ and R¹¹ are the same or different and are a fluorine atom or a hydrogen atom,

R⁸, R⁹ and R¹⁰ are the same or different and are a C1 to C6 alkyl group optionally having one or more halogen atoms, a C2 to C6 alkenyl group optionally having one or more halogen atoms, a C2 to C6 alkynyl group optionally having one or more halogen atoms, —OR¹², —S(O)ₘR¹², a halogen atom or a hydrogen atom,

R¹² is a C1 to C6 alkyl group optionally having one or more halogen atoms or a hydrogen atom, and

Q is an oxygen atom.

In the formula (1), compounds, wherein

R¹ is a C2 to C6 alkyl group, a cyclopropyl group or a cyclopropylmethyl group,

R², R⁴ and R⁵ are a hydrogen atom,

R³ is a C1 to C3 alkyl group optionally having one or more halogen atoms, a C1 to C3 alkoxy group optionally having one or more halogen atoms, a C1 to C3 alkylsulfanyl group optionally having one or more halogen atoms, a C1 to C3 alkylsulfinyl group optionally having one or more halogen atoms, a C1 to C3 alkylsulfonyl group optionally having one or more halogen atoms, a pyridyl group (wherein the pyridyl group optionally has one or more atoms or substituents selected from the group consisting of halogen atoms, C1 to C3 alkyl groups optionally having a halogen atom, and C1 to C3 alkoxy groups optionally having a halogen atom.), a pyrimidinyl group (wherein the pyrimidinyl group optionally has one or more atoms or substituents selected from the group consisting of halogen atoms, C1 to C3 alkyl groups optionally having a halogen atom, and C1 to C3 alkoxy groups optionally having a halogen atom.), a halogen atom or a hydrogen atom,

R⁶ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms or a C2 to C6 alkynyl group optionally having one or more halogen atoms,

R⁷ and R¹¹ are a hydrogen atom,

R⁸, R⁹ and R¹⁰ are the same or different and are a C1 to C2 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, a halogen atom or a hydrogen atom,

R¹² is a C1 to C6 alkyl group optionally having one or more halogen atoms or a hydrogen atom, and

Q is an oxygen atom.

In the formula (1), compounds, wherein

R¹ is an ethyl group, a cyclopropyl group or a cyclopropylmethyl group,

R², R⁴ and R⁵ are a hydrogen atom,

R⁵ is a C1 to C6 alkyl group optionally having one or more halogen atoms, a phenyl group optionally having one or more atoms or groups selected from group Z, a 5- or 6-membered heterocyclic group optionally having one or more atoms or groups selected from group Z, —OR¹², —S(O)ₘR¹⁴ or a halogen atom,

R⁶ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms or a C2 to C6 alkynyl group optionally having one or more halogen atoms,

R⁷ and R¹¹ are a hydrogen atom,

R⁸, R⁹ and R¹⁰ are the same or different and are a C1 to C6 perfluoroalkyl group, a C1 to C6 perfluoroalkoxy group, a C1 to C6 perfluoroalkylsulfanyl group, a C1 to C6 perfluoroalkylsulfinyl group, a C1 to C6 perfluoroalkylsulfonyl group, —SF⁵, a cyano group, a nitro group, a halogen atom, or a hydrogen atom,

R¹² is a C1 to C6 alkyl group optionally having one or more halogen atoms, a phenyl group optionally having one or more atoms or groups selected from group Z, a 5- or 6-membered heterocyclic group optionally having one or more atoms or groups selected from group Z or a hydrogen atom,

R¹⁴ is a C1 to C6 alkyl group optionally having one or more halogen atoms, a phenyl group optionally having one or more atoms or groups selected from group Z or a 5- or 6-membered heterocyclic group optionally having one or more atoms or groups selected from group Z, and

Q is an oxygen atom.

In the formula (1), compounds, wherein

R¹ is an ethyl group, a cyclopropyl group or a cyclopropylmethyl group,

R², R⁴ and R⁵ are a hydrogen atom,

R³ is a methyl group, an ethyl group, a vinyl group, a propyl group, an isopropyl group, a cyclopropyl group, a propargyl group, a trifluoromethyl group, a pentafluoroethyl group, a heptafluoropropyl group, a heptafluoroisopropyl group, a methoxy group, a trifluoromethoxy group, a methylsulfanyl group, a trifluoromethylsulfanyl group, a methylsulfinyl group, a trifluoromethylsulfinyl group, a methylsulfonyl group, a trifluoromethylsulfonyl group, an acetylamino group, a methoxycarbonylamino group, an ethoxycarbonylamino group, a tert-butoxycarbonylamino group, a methylsulfonylamino group, an amino group, an acetyl group, a methoxycarbonyl group, an ethoxycarbonyl group, a methylaminocarbonyl group, an ethylaminocarbonyl group, a dimethylaminocarbonyl group, a diethylaminocarbonyl group, an aminocarbonyl group, a 2-pyridyl group, a 2-pyrimidinyl group, a 5-trifluoromethyl-2-pyridyl group, a 3-chloro-5-trifluoromethyl-2-pyridyl group, a fluorine atom, a chlorine atom, a bromine atom, an iodine atom, or a hydrogen atom,

R⁶ is a methyl group, an ethyl group, a propyl group, an isopropyl group, a butyl group, an isobutyl group, a hexyl group, a propargyl group, a cyclopropyl group, a cyclobutyl group, a cyclohexyl group, a cyclopropylmethyl group, a cyclobutylmethyl group, a 2,2,2-trifluoroethyl group, a benzyl group, a 6-chloropyridin-3-ylmethyl group, a 2-chlorothiazolyl-5-ylmethyl group, a methoxymethyl group, an ethoxymethyl group, a methoxycarbonyl group, or an ethoxycarbonyl group,

R⁷ and R¹¹ are a hydrogen atom,

R⁸, R⁹ and R¹⁰ are the same or different and are a trifluoromethyl group, a pentafluoroethyl group, a heptafluoropropyl group, a heptafluoroisopropyl group, a trifluoromethoxy group, a trifluoromethylsulfanyl group, a trifluoromethylsulfinyl group or a trifluoromethylsulfonyl group, a fluorine atom, a chlorine atom, a bromine atom, or a hydrogen atom, and

Q is an oxygen atom.

In the formula (1), compounds, wherein

R¹ is an ethyl group, a cyclopropyl group or a cyclopropylmethyl group,

R², R⁴ and R⁵ are a hydrogen atom,

R³ is a methyl group, an ethyl group, a vinyl group, a propyl group, an isopropyl group, a cyclopropyl group, a propargyl group, a trifluoromethyl group, a pentafluoroethyl group, a heptafluoropropyl group, a heptafluoroisopropyl group, a methoxy group, a trifluoromethoxy group, a methylsulfanyl group, a trifluoromethylsulfanyl group, a methylsulfinyl group, a trifluoromethylsulfinyl group, a methylsulfonyl group, a trifluoromethylsulfonyl group, a 2-pyridyl group, a 2-pyrimidinyl group, a 5-trifluoromethyl-2-pyridyl group, a 3-chloro-5-trifluoromethyl-2-pyridyl group, a fluorine atom, a chlorine atom, a bromine atom, an iodine atom, or a hydrogen atom,

R⁶ is a methyl group, an ethyl group, a propyl group, an isopropyl group, a butyl group, an isobutyl group, a hexyl group, a propargyl group, a cyclopropyl group, a cyclobutyl group, a cyclohexyl group, a cyclopropylmethyl group, a cyclobutylmethyl group, a 2,2,2-trifluoroethyl group, a benzyl group, a 6-chloropyridin-3-ylmethyl group, a 2-chlorothiazolyl-5-ylmethyl group, a methoxymethyl group, an ethoxymethyl group, a methoxycarbonyl group, or an ethoxycarbonyl group,

R⁷ and R¹¹ are a hydrogen atom,

R⁸, R⁹ and R¹⁰ are the same or different and are a trifluoromethyl group, a pentafluoroethyl group, a heptafluoropropyl group, a heptafluoroisopropyl group, a trifluoromethoxy group, a trifluoromethylsulfanyl group, a trifluoromethylsulfinyl group or a trifluoromethylsulfonyl group, a fluorine atom, a chlorine atom, a bromine atom, or a hydrogen atom, and

Q is an oxygen atom.

In the formula (1), compounds wherein

R¹ is an ethyl group, a cyclopropyl group or a cyclopropylmethyl group,

R², R⁴ and R⁵ are a hydrogen atom,

R³ is a C1 to C3 perfluoroalkyl group, a C1 to C6 alkylcarbonylamino group optionally having one or more halogen atoms, a C1 to C6 alkoxycarbonylamino group optionally having one or more halogen atoms, a C2 to C6 alkoxycarbonyl group optionally having one or more halogen atoms, a C2 to C6 alkylaminocarbonyl group optionally having one or more halogen atoms, a halogen atom or a hydrogen atom,

R⁶ is a C1 to C3 alkyl group or a cyclopropylmethyl group,

R⁷ and R¹¹ are a hydrogen atom,

R⁸ and R¹⁰ are the same or different and are a C1 to C3 alkyl group optionally having one or more halogen atoms, a halogen atom or a hydrogen atom,

R⁹ is a C1 to C3 alkyl group optionally having one or more halogen atoms, a C1 to C3 alkoxy group optionally having one or more halogen atoms, a C1 to C3 alkylsulfanyl group optionally having one or more halogen atoms, a halogen atom or a hydrogen atom, and

Q is an oxygen atom.

In the formula (1), compounds wherein

R¹ is an ethyl group, a cyclopropyl group or a cyclopropylmethyl group,

R², R⁴ and R⁵ are a hydrogen atom,

R³ is a C1 to C3 perfluoroalkyl group, a halogen atom or a hydrogen atom,

R⁶ is a C1 to C3 alkyl group or a cyclopropylmethyl group,

R⁷ and R¹¹ are a hydrogen atom,

R⁸ and R¹⁰ are the same or different and are a C1 to C3 alkyl group optionally having one or more halogen atoms, a halogen atom or a hydrogen atom,

R⁹ is a C1 to C3 alkyl group optionally having one or more halogen atoms, a C1 to C3 alkoxy group optionally having one or more halogen atoms, a C1 to C3 alkylsulfanyl group optionally having one or more halogen atoms, a halogen atom or a hydrogen atom, and

Q is an oxygen atom.

Next, the method for producing the compound of the present invention will be described.

The compound of the present invention and an intermediate compound can be produced, for example, according to the following (Production Method 1) to (Production Method 13).

(Production Method 1)

The compound of the present invention (1-n1) or the compound of the present invention (1-n2) in which n is 1 or 2 in the formula (1) can be produced by oxidizing the compound of the present invention (1-n0) in which n is 0:

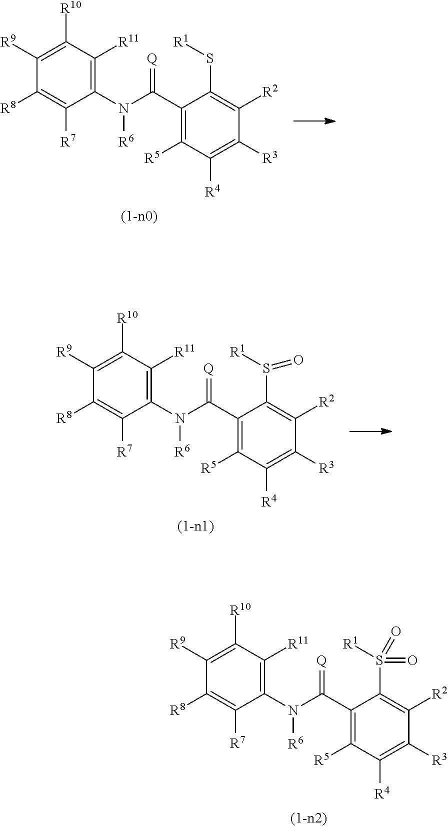

wherein symbols represent the same meaning as described above.

The compound of the present invention (1-n1) in which n is 1 in the formula (1) can be produced by subjecting the compound of the present invention (1-n0) in which n is 0 to an oxidation reaction.

The oxidation reaction is usually carried out in the presence of a solvent.

Examples of the solvent include aliphatic halogenated hydrocarbons such as dichloromethane and chloroform, alcohols such as methanol and ethanol, acetic acid, water, and mixtures thereof.

The oxidizing agent used in the oxidation reaction is, for example, sodium periodate or m-chloroperbenzoic acid.

In the reaction, the oxidizing agent is usually used in a ratio of 1 to 3 mol, based on 1 mol of the compound of the present invention (1-n0). Preferably, the oxidizing agent is used in a ratio of 1 to 1.2 mol, based on 1 mol of the compound of the present invention (1-n0).

The reaction temperature is usually within the range of −20 to 80° C. The reaction time is usually within the range of 0.1 to 12 hours.

After completion of the reaction, the reaction mixture is extracted with an organic solvent, and the organic layer is washed with an aqueous solution of a reducing agent (for example, sodium sulfite, sodium thiosulfate) and an aqueous solution of a base (for example, sodium bicarbonate) as necessary, and subjected to post-treatment operations such as drying and concentration, whereby the compound of the present invention (1-n1) can be isolated. The isolated compound of the present invention (1-n1) also can be further purified by chromatography, recrystallization, or the like. Alternatively, the compound of the present invention (1-n1) can be used in the next reaction as it is without isolation.

The compound of the present invention (1-n2) in which n is 2 in the formula (1) can be produced by subjecting the compound of the present invention (1-n1) in which n is 1 to an oxidation reaction.

The oxidation reaction is usually carried out in the presence of a solvent.

Examples of the solvent include aliphatic halogenated hydrocarbons such as dichloromethane and chloroform, alcohols such as methanol and ethanol, acetic acid, water, and mixtures thereof.

The oxidizing agent used in the oxidation reaction is, for example, m-chloroperbenzoic acid or aqueous hydrogen peroxide.

In the reaction, the oxidizing agent is usually used in a ratio of 1 to 4 mol, based on 1 mol of the compound of the present invention (1-n1). Preferably, the oxidizing agent is used in a ratio of 1 to 1.2 mol, based on 1 mol of the compound of the present invention (1-n1).

The reaction temperature is usually within the range of −20 to 120° C. The reaction time is usually within the range of 0.1 to 12 hours.

After completion of the reaction, the reaction mixture is extracted with an organic solvent, and the organic layer is washed with an aqueous solution of a reducing agent (for example, sodium sulfite, sodium thiosulfate) and an aqueous solution of a base (for example, sodium bicarbonate) as necessary, and subjected to post-treatment operations such as drying and concentration, whereby the compound of the present invention (1-n2) can be isolated. The compound of the present invention (1-n2) also can be further purified by chromatography, recrystallization, or the like.

The compound of the present invention (1-n2) in which n is 2 in the formula (1) can be produced by a one step reaction (one pot) by allowing the compound of the present invention (1-n0) in which n is 0 to react with an oxidizing agent.

The reaction is usually carried out in the presence of a solvent.

Examples of the solvent include aliphatic halogenated hydrocarbons such as dichloromethane and chloroform, alcohols such as methanol and ethanol, acetic acid, water, and mixtures thereof.

The oxidizing agent used in the reaction is, for example, m-chloroperbenzoic acid or aqueous hydrogen peroxide.

The reaction can be also carried out, in the presence of a catalyst, as necessary.

Examples of the catalyst used in the reaction include sodium tungstate.

In the reaction, the oxidizing agent is usually used in a ratio of 2 to 5 mol, and the catalyst is usually used in a ratio of 0.01 to 0.5 mol, based on 1 mol of the compound of the present invention (1-n0). Preferably, the oxidizing agent is used in a ratio of 2 to 2.2 mol, based on 1 mol of the compound of the present invention (1-n0).

The reaction temperature is usually within the range of 0 to 120° C. The reaction time is usually within the range of 0.1 to 12 hours.

After completion of the reaction, the reaction mixture is extracted with an organic solvent, and the organic layer is washed with an aqueous solution of a reducing agent (for example, sodium sulfite, sodium thiosulfate) and an aqueous solution of a base (for example, sodium bicarbonate) as necessary, and subjected to post-treatment operations such as drying and concentration, whereby the compound of the present invention (1-n2) can be isolated. The isolated compound of the present invention (1-n2) also can be further purified by chromatography, recrystallization, or the like.

(Production Method 2)

The compound of the present invention (P1) in which Q is an oxygen atom in the formula (1) can be produced by reacting the intermediate compound (M1) with the intermediate compound (M2):

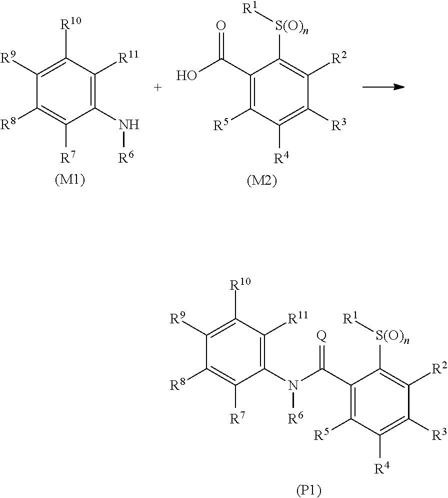

wherein symbols represent the same meaning as described above.

The compound of the present invention (P1) can be produced by reacting the intermediate compound (M1) with the intermediate compound (M2), in the presence of a condensing agent.

The reaction is usually carried out in the presence of a solvent. Examples of the solvent include ethers such as 1,4-dioxane, diethyl ether, tetrahydrofuran (hereinafter, referred to as THF) and tert-butyl methyl ether, halogenated hydrocarbons such as dichloromethane, chloroform, carbon tetrachloride, 1,2-dichloroethane and chlorobenzene, aromatic hydrocarbons such as toluene, benzene and xylene, esters such as ethyl acetate and butyl acetate, nitriles such as acetonitrile, aprotic polar solvents such as N,N-dimethylformamide (hereinafter, referred to as DMF), N-methyl pyrrolidone (hereinafter, referred to as NMP), 1,3-dimethyl-2-imidazolidinone and dimethyl sulfoxide (hereinafter, referred to as DMSO), nitrogen-containing aromatic compounds such as pyridine and quinolone, and mixtures thereof.

Examples of the condensing agent include carbodiimides such as 1-ethyl-3-(3-dimethylaminopropyl) carbodiimide hydrochloride (hereinafter, referred to as EDCI hydrochloride) and 1,3-dicyclohexylcarbodiimide.

The reaction can be also carried out by adding a catalyst, as necessary. Examples of the catalyst include 1-hydroxybenzotriazole (hereinafter, referred to as HOBt).

In the reaction, the intermediate compound (M2) is usually used in a ratio of 0.8 to 1.2 mol, the condensing agent is usually used in a ratio of 1 to 2 mol, and the catalyst is usually used in a ratio of 0.01 to 1 mol, based on 1 mol of the intermediate compound (M1).

The reaction temperature is usually within the range of 0 to 200° C. The reaction time is usually within the range of 0.1 to 24 hours.

After completion of the reaction, the compound of the present invention (P1) can be isolated by adding the reaction mixture to water, then extracting the mixture with an organic solvent, and concentrating the organic layer; collecting a solid generated by adding the reaction mixture to water by filtration; or collecting a solid generated in the reaction mixture by filtration. The isolated compound of the present invention (P1) also can be further purified by recrystallization, chromatography, or the like.

(Production Method 3)

The compound of the present invention (P1) in which Q is an oxygen atom in the formula (1) can be produced by reacting the intermediate compound (M1) with the intermediate compound (M3):

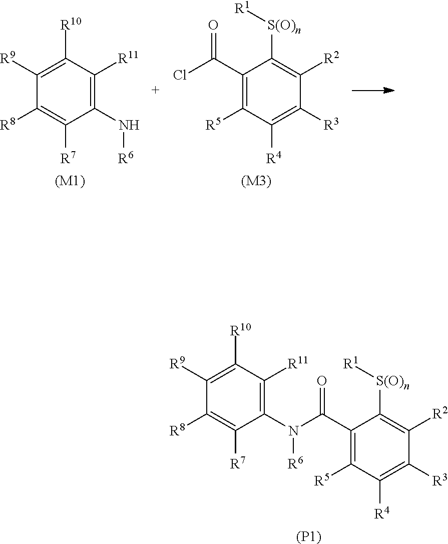

wherein symbols represent the same meaning as described above.

The compound of the present invention (P1) can be produced by reacting the compound (M1) with the intermediate compound (M3).

The reaction is usually carried out in the presence of a solvent.

Examples of the solvent include ethers such as THF, ethylene glycol dimethyl ether, tert-butyl methyl ether and 1,4-dioxane, aliphatic hydrocarbons such as hexane, heptane and octane, aromatic hydrocarbons such as toluene and xylene, halogenated hydrocarbons such as chlorobenzene, esters such as ethyl acetate and butyl acetate, nitriles such as acetonitrile, aprotic polar solvents such as DMF, NMP, and DMSO, and mixtures thereof.

The reaction can be also carried out by adding a base, as necessary. The base includes alkali metal carbonates such as sodium carbonate and potassium carbonate, tertiary amines such as triethylamine and N,N-diisopropylethylamine, and nitrogen-containing aromatic compounds such as pyridine, 4-dimethylaminopyridine, and the like.

In the reaction, the intermediate compound (M3) is usually used in a ratio of 0.8 to 1.2 mol, and the base is usually used in a ratio of 1 to 2 mol, based on 1 mol of the intermediate compound (M1).

The reaction temperature is usually within the range of −20 to 200° C. The reaction time is usually within the range of 0.1 to 24 hours.

After completion of the reaction, water is added to the reaction mixture, then the reaction mixture is extracted with an organic solvent, and the organic layer is subjected to post-treatment operations such as drying and concentration, whereby the compound of the present invention (P1) can be isolated. The isolated compound of the present invention (P1) also can be further purified by chromatography, recrystallization, or the like.

(Production Method 4)

The compound of the present invention (P1) in which Q is an oxygen atom in the formula (1) can be produced by reacting the intermediate compound (M4) with the intermediate compound (M5):

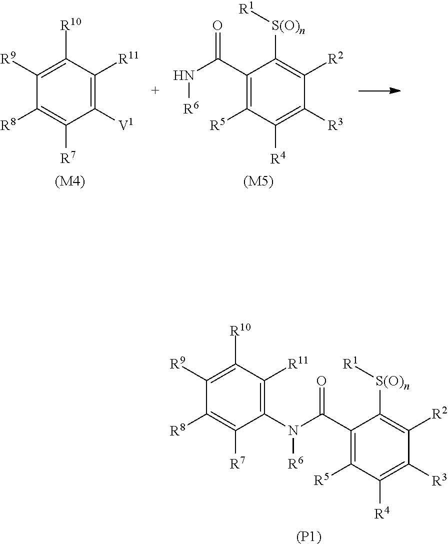

wherein V¹ represents a halogen atom, and other symbols represent the same meaning as described above.

When V¹ is a fluorine atom, the compound of the present invention (P1) in which Q is an oxygen atom in the formula (1) can be produced by reacting the intermediate compound (M4) with the intermediate compound (M5), in the presence of a base.

The reaction is usually carried out in the presence of a solvent.

Examples of the solvent include ethers such as THF, ethylene glycol dimethyl ether, tert-butyl methyl ether and 1,4-dioxane, aromatic hydrocarbons such as toluene and xylene, nitriles such as acetonitrile, aprotic polar solvents such as DMF, NMP and DMSO, water, and mixtures thereof.

Examples of the base include alkali metal carbonates such as sodium carbonate, cesium carbonate and potassium carbonate, and alkali metal hydrides such as sodium hydride.

In the reaction, the intermediate compound (M5) is usually used in a ratio of 0.8 to 1.2 mol, and the base is usually used in a ratio of 1 to 2 mol, based on 1 mol of the intermediate compound (M4).

The reaction temperature is usually within the range of 0 to 200° C. The reaction time is usually within the range of 0.5 to 24 hours.

After completion of the reaction, the reaction mixture is extracted with an organic solvent, and the organic layer is subjected to post-treatment operations such as drying and concentration, whereby the compound of the present invention (P1) can be isolated. The isolated compound of the present invention (P1) also can be further purified by chromatography, recrystallization, or the like.

When V¹ is a chlorine atom, a bromine atom or an iodine atom, the compound of the present invention (P1) in which Q is an oxygen atom in the formula (1) can be produced by reacting the intermediate compound (M4) with the intermediate compound (M5) and a base, in the presence of a copper catalyst or a palladium catalyst.

The reaction is usually carried out in the presence of a solvent.

Examples of the solvent include ethers such as THF, ethylene glycol dimethyl ether, tert-butyl methyl ether and 1,4-dioxane, aromatic hydrocarbons such as toluene and xylene, nitriles such as acetonitrile, aprotic polar solvents such as DMF, NMP and DMSO, water, and mixtures thereof.

Examples of the base include alkali metal carbonates such as sodium carbonate and potassium carbonate, phosphates such as tri sodium phosphate and tripotassium phosphate, alkali metal hydrides such as sodium hydride, tertiary amines such as triethylamine and N,N-diisopropylethylamine, and cyclic amines such as 1,4-diazabicyclo[2.2.2]octane (hereinafter, referred to as DABCO) and diazabicycloundecene (hereinafter, referred to as DBU).

Examples of the copper catalyst include copper(I) iodide, copper(I) bromide, copper(I) chloride, and copper(I) oxide.

Examples of the palladium catalyst include palladium(II)acetate and tris(dibenzylideneacetone)dipalladium(O).

The reaction can be also carried out by adding a ligand as necessary. Examples of the ligand include acetylacetone, salen, phenanthroline, triphenylphosphine, and 4,5′-bis(diphenylphosphino)-9,9′-dimethylxanthene.

In the reaction, the intermediate compound (M5) is usually used in a ratio of 0.8 to 1.2 mol, the base is usually used in a ratio of 1 to 2 mol, the copper catalyst is usually used in a ratio of 0.01 to 0.5 mol, the palladium catalyst is usually used in a ratio of 0.01 to 0.2 mol, and the ligand is usually used in a ratio of 0.01 to 0.5 mol, based on 1 mol of the intermediate compound (M4).

The reaction temperature is usually within the range of 0 to 200° C. The reaction time is usually within the range of 0.5 to 24 hours.

After completion of the reaction, the reaction mixture is extracted with an organic solvent, and the organic layer is subjected to post-treatment operations such as drying and concentration, whereby the compound of the present invention (P1) can be isolated. The isolated compound of the present invention (P1) also can be further purified by chromatography, recrystallization, or the like.

(Production Method 5)

The compound of the present invention (1) can be produced by reacting the intermediate compound (M6) with the intermediate compound (M7), in the presence of a base:

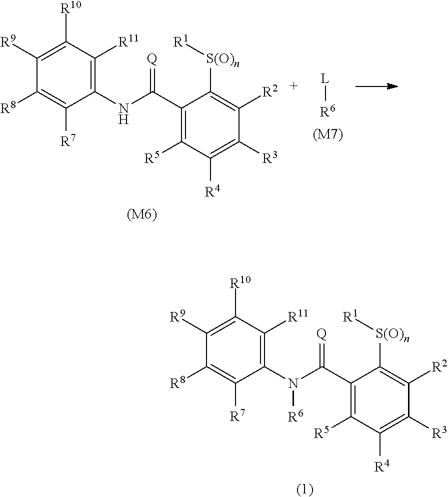

wherein L represents a leaving group such as a chlorine atom, a bromine atom, an iodine atom, a trifluoromethanesulfonyloxy group or a methanesulfonyloxy group, and other symbols represent the same meaning as described above.

The reaction is usually carried out in the presence of a solvent.

Examples of the solvent include ethers such as THF, ethylene glycol dimethyl ether, tert-butyl methyl ether and 1,4-dioxane, aromatic hydrocarbons such as toluene and xylene, nitriles such as acetonitrile, aprotic polar solvents such as DMF, NMP and DMSO, and mixtures thereof.

Examples of the base include hydrides of alkali metals or alkaline earth metals such as sodium hydride, potassium hydride and calcium hydride, inorganic bases such as sodium carbonate and potassium carbonate, organic bases such as triethylamine, and the like.

In the reaction, the intermediate compound (M7) is usually used in a ratio of 1 to 5 mol, and the base is usually used in a ratio of 1 to 2 mol, based on 1 mol of the intermediate compound (M6).

The reaction temperature is usually within the range of 0 to 100° C. The reaction time is usually within the range of 0.1 to 24 hours.

After completion of the reaction, the reaction mixture is extracted with an organic solvent, and the organic layer is subjected to post-treatment operations such as drying and concentration, whereby the compound of the present invention (1) can be isolated. The isolated compound of the present invention (1) also can be further purified by chromatography, recrystallization, or the like.

(Production Method 6)

The compound of the present invention (1) can be produced by reacting the intermediate compound (M6) with the intermediate compound (M8), in the presence of a condensing agent:

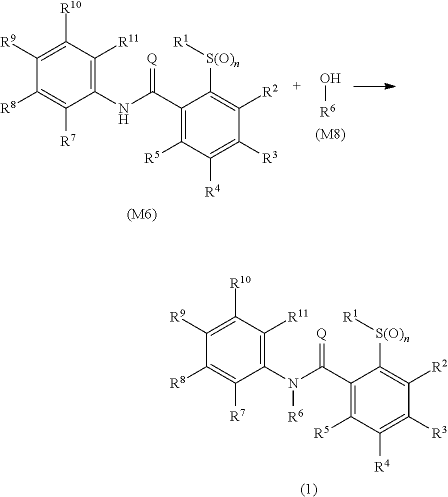

wherein symbols represent the same meaning as described above.

The reaction is usually carried out in the presence of a solvent.

Examples of the solvent include ethers such as THF, ethylene glycol dimethyl ether, tert-butyl methyl ether and 1,4-dioxane, aromatic hydrocarbons such as toluene and xylene, nitriles such as acetonitrile, aliphatic halogenated hydrocarbons such as dichloromethane and chloroform, aprotic polar solvents such as DMF, NMP and DMSO, and mixtures thereof.

Examples of the condensing agent include a mixture of diethyl azodicarboxylate and triphenylphosphine.

In the reaction, the intermediate compound (Me) is usually used in a ratio of 1 to 5 mol, and the condensing agent is usually used in a ratio of 1 to 2 mol, based on 1 mol of the intermediate compound (M6).

The reaction temperature is usually within the range of 0 to 150° C. The reaction time is usually within the range of 0.1 to 24 hours.

After completion of the reaction, the reaction mixture is extracted with an organic solvent, and the organic layer is subjected to post-treatment operations such as drying and concentration, whereby the compound of the present invention (1) can be isolated. The isolated compound of the present invention (1) also can be further purified by chromatography, recrystallization, or the like.

(Production Method 7)

The compound of the present invention (P2) in which Q is an oxygen atom and n is 0 in the formula (1) can be produced by reacting the intermediate compound (M11) with the intermediate compound (M12), in the presence of a base:

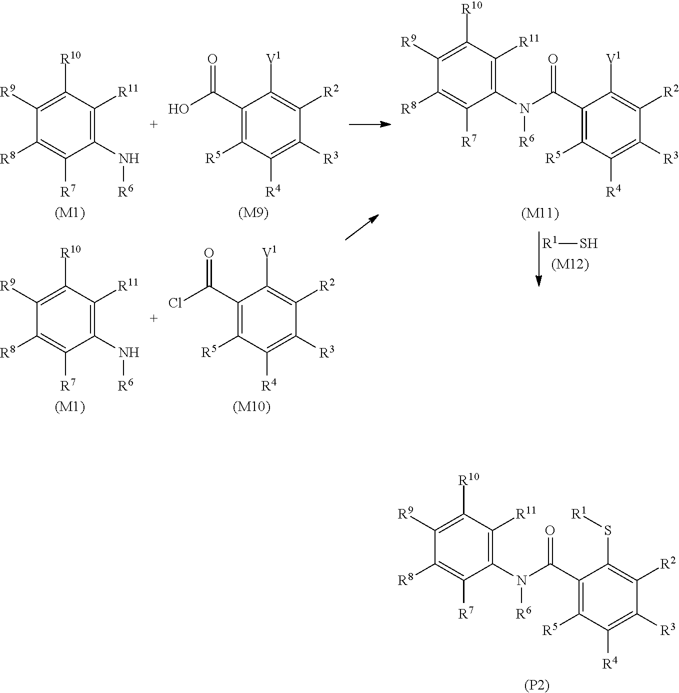

wherein symbols represent the same meaning as described above.

The intermediate compound (M11) can be produced, using the intermediate compound (M9) in place of the intermediate compound (M2), in accordance with the method of Production Method 2.

The intermediate compound (M11) can be produced, using the intermediate compound (M10) in place of the intermediate compound (M2), in accordance with the method of Production Method 3.

The compound of the present invention (P2) in which Q is an oxygen atom and n is 0 in the formula (1) can be produced by reacting the intermediate compound (M11) with the intermediate compound (M12), in the presence of a base.

The reaction is usually carried out in the presence of a solvent.

Examples of the solvent include ethers such as THF, ethylene glycol dimethyl ether, tert-butyl methyl ether and 1,4-dioxane, aromatic hydrocarbons such as toluene and xylene, nitriles such as acetonitrile, aprotic polar solvents such as DMF, NMP and DMSO, water, and mixtures thereof.

Examples of the base include alkali metal carbonates such as sodium carbonate and potassium carbonate, and alkali metal hydrides such as sodium hydride.

In the reaction, the compound (M12) is usually used in a ratio of 0.8 to 1.2 mol, and the base is usually used in a ratio of 1 to 2 mol, based on 1 mol of the intermediate compound (M11).

The reaction temperature is usually within the range of 0 to 150° C. The reaction time is usually within the range of 0.5 to 24 hours.

After completion of the reaction, the reaction mixture is extracted with an organic solvent, and the organic layer is subjected to post-treatment operations such as drying and concentration, whereby the compound of the present invention (P2) can be isolated. The isolated compound of the present invention (P2) also can be further purified by chromatography, recrystallization, or the like.

In the reaction, V¹ is preferably a fluorine atom or a chlorine atom.

(Production Method 8)

The compound of the present invention (P2) in which Q is an oxygen atom and n is 0 in the formula (1) can be produced by producing the intermediate compound (M13) using sodium sulfide, sodium hydrogen sulfide or hydrogen sulfide in place of the intermediate compound (M12), in accordance with the method of Production Method 7, and reacting the intermediate compound (M13) with the intermediate compound (M14), in the presence of a base:

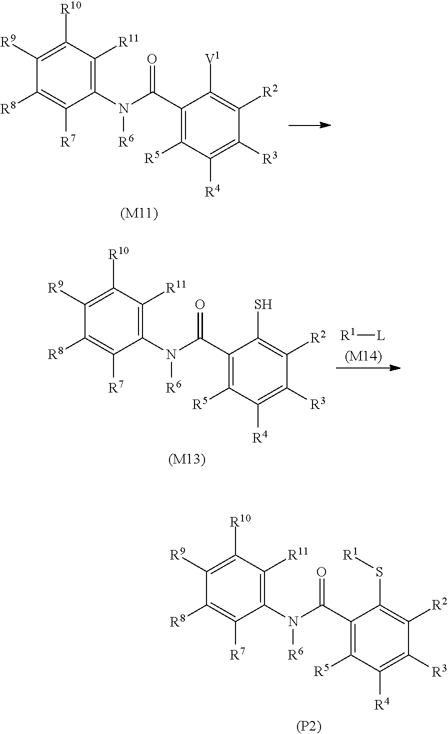

wherein symbols represent the same meaning as described above.

The intermediate compound (M13) can be produced, using sodium sulfide, sodium hydrogen sulfide or hydrogen sulfide in place of the intermediate compound (M12), in accordance with the method of Production Method 7. Here, when using sodium sulfide or sodium hydrogen sulfide in place of the intermediate compound (M12), the intermediate compound (M13) can be usually produced by the reaction in the absence of a base. Also, when using hydrogen sulfide in place of the intermediate compound (M12), the intermediate compound (M13) can be usually produced by the reaction in the presence of a base.

The compound of the present invention (P2) in which Q is an oxygen atom and n is 0 in the formula (1) can be produced by reacting the intermediate compound (M13) with the intermediate compound (M14), in the presence of a base.

The reaction is usually carried out in the presence of a solvent.

Examples of the solvent include ethers such as THF, ethylene glycol dimethyl ether, tert-butyl methyl ether and 1,4-dioxane, aromatic hydrocarbons such as toluene and xylene, nitriles such as acetonitrile, aprotic polar solvents such as DMF, NMP and DMSO, and mixtures thereof.

Examples of the base include hydrides of alkali metals or alkaline earth metals such as sodium hydride, potassium hydride and calcium hydride, inorganic bases such as sodium carbonate and potassium carbonate, and organic bases such as triethylamine.

In the reaction, the intermediate compound (M14) is usually used in a ratio of 1 to 5 mol, and the base is usually used in a ratio of 1 to 2 mol, based on 1 mol of the intermediate compound (M13).

The reaction temperature is usually within the range of 0 to 100° C. The reaction time is usually within the range of 0.1 to 24 hours.

After completion of the reaction, the reaction mixture is extracted with an organic solvent, and the organic layer is subjected to post-treatment operations such as drying and concentration, whereby the compound of the present invention (P2) can be isolated. The isolated compound of the present invention (P2) also can be further purified by chromatography, recrystallization, or the like.

(Production Method 9)

The compound of the present invention (P3) in which Q is a sulfur atom in the formula (1) can be produced by reacting the compound of the present invention (P1) in which Q is an oxygen atom in the formula (1) with a sulfurizing agent:

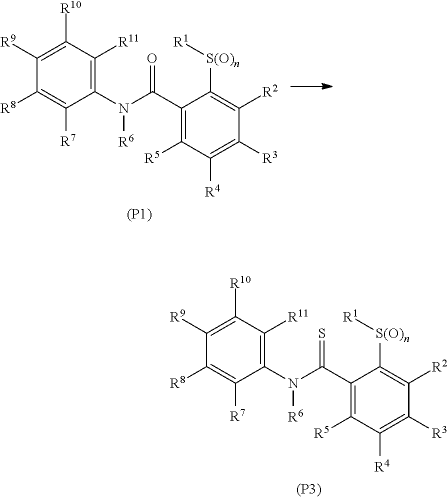

wherein symbols represent the same meaning as described above.

The reaction is usually carried out in the presence or absence of a solvent.

Examples of the solvent include ethers such as 1,4-dioxane, diethyl ether, tetrahydrofuran, tert-butyl methyl ether and diglyme, halogenated hydrocarbons such as dichloromethane, chloroform, carbon tetrachloride, 1,2-dichloroethane and chlorobenzene, aromatic hydrocarbons such as toluene, benzene and xylene, nitriles such as acetonitrile, nitrogen-containing aromatic compounds such as pyridine, picoline, lutidine and quinoline, and mixtures thereof.

The sulfurizing agent includes diphosphorus pentasulfide, Lawesson's reagent (2,4-bis-(4-methoxyphenyl)-1,3-dithia-2,4-diphosphetane-2,4-disulfide), and the like.

In the reaction, the sulfurizing agent is usually used in a ratio of 1 to 3 mol, based on 1 mol of the compound of the present invention (P1).

The reaction temperature is usually within the range of 0° C. to 200° C., and the reaction time is usually within the range of 1 to 24 hours.

After completion of the reaction, the compound of the present invention (P3) can be isolated by adding the reaction mixture to water, then extracting the mixture with an organic solvent, and concentrating the organic layer; collecting a solid generated by adding the reaction mixture to water by filtration; or collecting a solid generated in the reaction mixture by filtration. The isolated compound of the present invention (P3) also can be further purified by recrystallization, chromatography, or the like.

(Production Method 10)

The intermediate compound (M2) can be produced by hydrolyzing the intermediate compound (M15). Also, the intermediate compound (M3) can be produced by reacting the intermediate compound (M2) with a chlorinating agent:

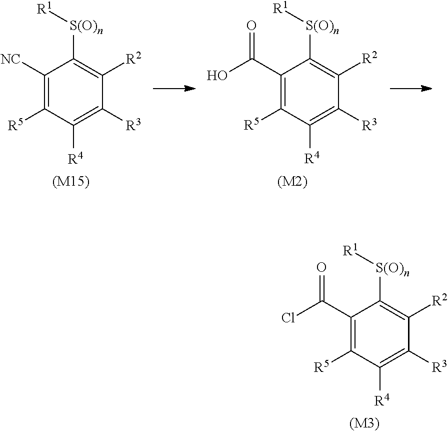

wherein symbols represent the same meaning as described above.

The intermediate compound (M2) can be produced by hydrolyzing the intermediate compound (M15).

When hydrolyzed by an acid, the hydrolysis is usually carried out using an aqueous solution of an acid as a solvent.

Examples of the acid used in the hydrolysis include mineral acids such as hydrochloric acid, nitric acid, phosphoric acid and sulfuric acid, carboxylic acids such as acetic acid and trifluoroacetic acid.

The reaction temperature is usually within the range of 0 to 150° C. The reaction time is usually within the range of 0.1 to 24 hours.

After completion of the reaction, the reaction mixture is extracted with an organic solvent, and the organic layer is subjected to post-treatment operations such as drying and concentration, whereby the intermediate compound (M2) can be isolated. The isolated intermediate compound (M2) also can be further purified by chromatography, recrystallization, or the like.

When hydrolyzed by a base, the hydrolysis is usually carried out in the presence of a solvent.

Examples of the solvent include ethers such as THF, ethylene glycol dimethyl ether, tert-butyl methyl ether and 1,4-dioxane, alcohols such as methanol and ethanol, water, and mixtures thereof.

Examples of the base include alkali metal hydroxides such as sodium hydroxide and potassium hydroxide.

In the hydrolysis, the base is usually used in a ratio of 1 to 10 mol, based on 1 mol of the intermediate compound (M15).

The reaction temperature is usually within the range of 0 to 120° C. The reaction time is usually within the range of 0.1 to 24 hours.

After completion of the reaction, the reaction solution is acidified, then the reaction mixture is extracted with an organic solvent, and the organic layer is subjected to post-treatment operations such as drying and concentration, whereby the intermediate compound (M2) can be isolated. The isolated intermediate compound (M2) also can be further purified by chromatography, recrystallization, or the like.

The intermediate compound (M3) can be produced by allowing the intermediate compound (M2) to react with a chlorinating agent.

The reaction is usually carried out in the presence of a solvent.

Examples of the solvent include ethers such as THF, ethylene glycol dimethyl ether, tert-butyl methyl ether and 1,4-dioxane, aromatic hydrocarbons such as toluene and xylene, aliphatic halogenated hydrocarbons such as dichloromethane and chloroform, and mixtures thereof.

The chlorinating agent includes thionyl chloride, oxalyl dichloride, phosphorus oxychloride, and the like.

The reaction can be also carried out by adding DMF as necessary.

In the reaction, the chlorinating agent is usually used in a ratio of 1 to 5 mol, and DMF is usually used in a ratio of 0.001 to 0.1 mol, based on 1 mol of the intermediate compound (M2).

The reaction temperature is usually within the range of 0 to 100° C. The reaction time is usually within the range of 0.1 to 24 hours.

After completion of the reaction, the intermediate compound (M3) can be isolated by distilling the solvent. Alternatively, the intermediate compound (M3) can be used in the next reaction as it is without isolation.

(Production Method 11)

The intermediate compound (M2-n0) in which n is 0 in the intermediate compound (M2) can be produced by reacting the intermediate compound (M16) with the intermediate compound (M12), in the presence of a base. Also, the intermediate compound (M2-n1) or intermediate compound (M2-n2) in which n is 1 or 2 in the intermediate compound (M2) can be produced by oxidizing the intermediate compound (M2) in which n is 0:

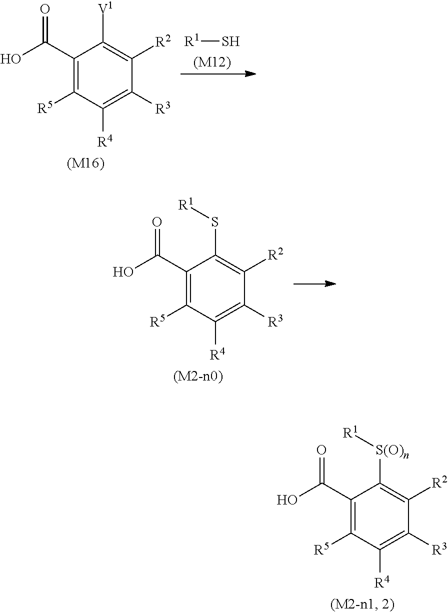

wherein symbols represent the same meaning as described above.

The intermediate compound (M2-n0) in which n is 0 can be produced, using the intermediate compound (M16) in place of the intermediate compound (M11), in accordance with the method of Production Method 7.

In the reaction, V¹ is preferably a fluorine atom or a chlorine atom.

The intermediate compound (M2-n1) or intermediate compound (M2-n2) in which n is 1 or 2 can be produced, using the intermediate compound (M2-n0) in which n is 0 in place of the compound of the present invention (1-n0) in which n is 0, in accordance with the method of Production Method 1.

(Production Method 12)

The intermediate compound (M6-QO) in which Q is an oxygen atom in the intermediate compound (M6) can be produced, using the intermediate compound (M17) in place of the intermediate compound (M1), in accordance with the method of Production Method 2. Also, the intermediate compound (M6) can be produced, using the intermediate compound (M17) in place of the intermediate compound (M1), in accordance with the method of Production Method 3.

The intermediate compound (M6-QS) in which Q is a sulfur atom in the intermediate compound (M6) can be produced, using the intermediate compound (M6-QO) in which Q is an oxygen atom in place of the compound of the present invention (P1), in accordance with the method of Production Method 9:

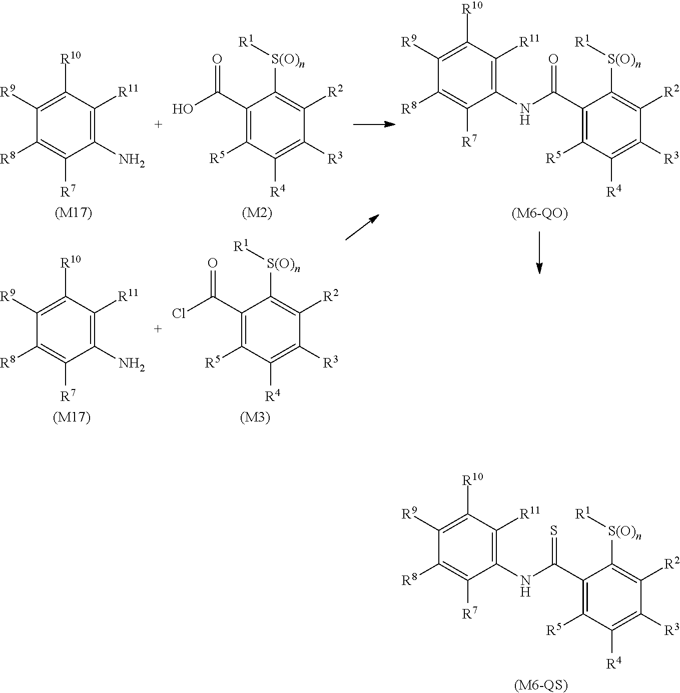

wherein symbols represent the same meaning as described above.

(Production Method 13)

Among the compounds of the present invention and the above-described intermediate compounds, a compound having a nitrogen-containing heterocyclic group having a lone pair of electrons on the nitrogen atom is reacted with an oxidizing agent, whereby an N-oxide in which the nitrogen atom is oxidized can be manufactured in some cases.

Examples of the nitrogen-containing heterocyclic group include a pyridine ring.

The reaction can be carried out by a known method, and is carried out using an oxidizing agent such as m-chloroperbenzoic acid or hydrogen peroxide, in a solvent, for example, a halogenated hydrocarbon such as dichloromethane, chloroform or chlorobenzene, an alcohol such as methanol or ethanol, acetic acid, water, and mixtures thereof.

Examples of the intermediate compound (M6) include following compounds.

Compounds represented by formula (M6):

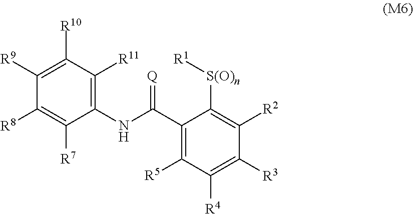

wherein symbols represent the same meaning as described above, or N-oxides thereof.

In the formula (M6), compounds, wherein

R¹ is an ethyl group, a cyclopropyl group or a cyclopropylmethyl group,

R², R⁴ and R⁵ are a hydrogen atom,

R³ is a C1 to C6 alkyl group optionally having one or more halogen atoms, a phenyl group optionally having one or more atoms or groups selected from group Z, a 5- or 6-membered heterocyclic group optionally having one or more atoms or groups selected from group Z, —OR¹², —S(O)ₘR¹⁴—NR¹²R¹³, —NR¹²C(O)R¹³, —NR¹²CO₂R¹³, —NR¹²S(O)₂R¹⁴, —C(O)R¹², —CO₂R¹², —C(O)NR¹²R¹³, a halogen atom or a hydrogen atom,

R⁷ and R¹¹ are a hydrogen atom,

R⁸, R⁹ and R¹⁰ are the same or different and are a C1 to C6 perfluoroalkyl group, a C1 to C6 perfluoroalkoxy group, a C1 to C6 perfluoroalkylsulfanyl group, a C1 to C6 perfluoroalkylsulfinyl group, a C1 to C6 perfluoroalkylsulfonyl group, —SF₅, a cyano group, a nitro group, a halogen atom, or a hydrogen atom (wherein R⁸, R⁹ and R¹⁰ do not simultaneously represent a hydrogen atom),

R¹² is a C1 to C6 alkyl group optionally having one or more halogen atoms, a phenyl group optionally having one or more atoms or groups selected from group Z, a 5- or 6-membered heterocyclic group optionally having one or more atoms or groups selected from group Z or a hydrogen atom,

R¹⁴ is a C1 to C6 alkyl group optionally having one or more halogen atoms, a phenyl group optionally having one or more atoms or groups selected from group Z or a 5- or 6-membered heterocyclic group optionally having one or more atoms or groups selected from group Z,

Q is an oxygen atom or a sulfur atom,

m is 0, 1 or 2, and

n is 0, 1 or 2.

In the formula (M6), compounds, wherein

R¹ is an ethyl group, a cyclopropyl group or a cyclopropylmethyl group,

R², R⁴ and R⁵ are a hydrogen atom,

R³ is a C1 to C6 alkyl group optionally having one or more halogen atoms, a phenyl group optionally having one or more atoms or groups selected from group Z, a 5- or 6-membered heterocyclic group optionally having one or more atoms or groups selected from group Z, —OR¹², —S(O)ₘR¹⁴ or a halogen atom,

R⁷ and R¹¹ are a hydrogen atom,

R⁸, R⁹ and R¹⁰ are the same or different and are a C1 to C6 perfluoroalkyl group, a C1 to C6 perfluoroalkoxy group, a C1 to C6 perfluoroalkylsulfanyl group, a C1 to C6 perfluoroalkylsulfinyl group, a C1 to C6 perfluoroalkylsulfonyl group, —SF₅, a cyano group, a nitro group, a halogen atom, or a hydrogen atom (wherein R⁸, R⁹ and R¹⁰ do not simultaneously represent a hydrogen atom),

R¹² is a C1 to C6 alkyl group optionally having one or more halogen atoms, a phenyl group optionally having one or more atoms or groups selected from group Z, a 5- or 6-membered heterocyclic group optionally having one or more atoms or groups selected from group Z or a hydrogen atom,

R¹⁴ is a C1 to C6 alkyl group optionally having one or more halogen atoms, a phenyl group optionally having one or more atoms or groups selected from group Z or a 5- or 6-membered heterocyclic group optionally having one or more atoms or groups selected from group Z,

Q is an oxygen atom or a sulfur atom,

m is 0, 1 or 2, and

n is 0, 1 or 2.

In the formula (M6), compounds wherein Q is an oxygen atom.

In the formula (M6), compounds, wherein

R¹ is an ethyl group, a cyclopropyl group or a cyclopropylmethyl group,

R², R⁴ and R⁵ are a hydrogen atom,

R³ is a methyl group, an ethyl group, a vinyl group, a propyl group, an isopropyl group, a cyclopropyl group, a propargyl group, a trifluoromethyl group, a pentafluoroethyl group, a heptafluoropropyl group, a heptafluoroisopropyl group, a methoxy group, a trifluoromethoxy group, a methylsulfanyl group, a trifluoromethylsulfanyl group, a methylsulfinyl group, a trifluoromethylsulfinyl group, a methylsulfonyl group, a trifluoromethylsulfonyl group, a 2-pyridyl group, a 2-pyrimidinyl group, a 5-trifluoromethyl-2-pyridyl group, a 3-chloro-5-trifluoromethyl-2-pyridyl group, a fluorine atom, a chlorine atom, a bromine atom, or an iodine atom,

R⁷ and R¹¹ are a hydrogen atom,

R⁸, R⁹ and R¹⁰ are the same or different and are a trifluoromethyl group, a pentafluoroethyl group, a heptafluoropropyl group, a heptafluoroisopropyl group, a trifluoromethoxy group, a trifluoromethylsulfanyl group, a trifluoromethylsulfinyl group or a trifluoromethylsulfonyl group, a fluorine atom, a chlorine atom, a bromine atom, or a hydrogen atom (wherein R⁸, R⁹ and R¹⁰ do not simultaneously represent a hydrogen atom), and

Q is an oxygen atom.

Next, specific examples of the compound of the present invention are shown below.

In the formula (1):

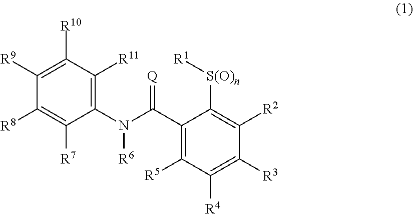

wherein n is 0, R², R³, R⁴, R⁵, R⁷ and R¹¹ are a hydrogen atom, Q is an oxygen atom, and R¹, R⁶, R⁸, R⁹ and R¹⁰ are the combinations shown in [Table 1] to [Table 24].

TABLE 1

| R¹ | R⁶ | R⁸ | R⁹ | R¹⁰ |
|---|---|---|---|---|
| Me | Me | H | CF₃ | H |
| Et | Me | H | CF₃ | H |
| Pr | Me | H | CF₃ | H |
| CH₂CF₃ | Me | H | CF₃ | H |
| CH₂C≡CH | Me | H | CF₃ | H |
| CycPr | Me | H | CF₃ | H |
| CH₂CycPr | Me | H | CF₃ | H |
| Me | Et | H | CF₃ | H |
| Et | Et | H | CF₃ | H |
| Pr | Et | H | CF₃ | H |
| CH₂CF₃ | Et | H | CF₃ | H |
| CH₂C≡CH | Et | H | CF₃ | H |
| CycPr | Et | H | CF₃ | H |
| CH₂CycPr | Et | H | CF₃ | H |
| Me | CycPr | H | CF₃ | H |
| Et | CycPr | H | CF₃ | H |
| Pr | CycPr | H | CF₃ | H |
| CH₂CF₃ | CycPr | H | CF₃ | H |
| CH₂C≡CH | CycPr | H | CF₃ | H |
| CycPr | CycPr | H | CF₃ | H |
| CH₂CycPr | CycPr | H | CF₃ | H |

TABLE 2

| R¹ | R⁶ | R⁸ | R⁹ | R¹⁰ |
|---|---|---|---|---|
| Me | CH₂CycPr | H | CF₃ | H |
| Et | CH₂CycPr | H | CF₃ | H |
| Pr | CH₂CycPr | H | CF₃ | H |
| CH₂CF₃ | CH₂CycPr | H | CF₃ | H |
| CH₂C≡CH | CH₂CycPr | H | CF₃ | H |
| CycPr | CH₂CycPr | H | CF₃ | H |
| CH₂CycPr | CH₂CycPr | H | CF₃ | H |
| Me | CH₂C≡CH | H | CF₃ | H |
| Et | CH₂C≡CH | H | CF₃ | H |
| Pr | CH₂C≡CH | H | CF₃ | H |
| CH₂CF₃ | CH₂C≡CH | H | CF₃ | H |
| CH₂C≡CH | CH₂C≡CH | H | CF₃ | H |
| CycPr | CH₂C≡CH | H | CF₃ | H |
| CH₂CycPr | CH₂C≡CH | H | CF₃ | H |
| Me | CH₂OCH₃ | H | CF₃ | H |
| Et | CH₂OCH₃ | H | CF₃ | H |
| Pr | CH₂OCH₃ | H | CF₃ | H |
| CH₂CF₃ | CH₂OCH₃ | H | CF₃ | H |
| CH₂C≡CH | CH₂OCH₃ | H | CF₃ | H |
| CycPr | CH₂OCH₃ | H | CF₃ | H |
| CH₂CycPr | CH₂OCH₃ | H | CF₃ | H |

TABLE 3

| R¹ | R⁶ | R⁸ | R⁹ | R¹⁰ |
|---|---|---|---|---|
| Me | CO₂CH₃ | H | CF₃ | H |
| Et | CO₂CH₃ | H | CF₃ | H |
| Pr | CO₂CH₃ | H | CF₃ | H |
| CH₂CF₃ | CO₂CH₃ | H | CF₃ | H |
| CH₂C≡CH | CO₂CH₃ | H | CF₃ | H |
| CycPr | CO₂CH₃ | H | CF₃ | H |
| CH₂CycPr | CO₂CH₃ | H | CF₃ | H |
| Me | 6-chloro-pyridin-3-ylmethyl | H | CF₃ | H |
| Et | 6-chloro-pyridin-3-ylmethyl | H | CF₃ | H |
| Pr | 6-chloro-pyridin-3-ylmethyl | H | CF₃ | H |
| CH₂CF₃ | 6-chloro-pyridin-3-ylmethyl | H | CF₃ | H |
| CH₂C≡CH | 6-chloro-pyridin-3-ylmethyl | H | CF₃ | H |
| CycPr | 6-chloro-pyridin-3-ylmethyl | H | CF₃ | H |
| CH₂CycPr | 6-chloro-pyridin-3-ylmethyl | H | CF₃ | H |
| Me | 2-chloro-thiazol-5-ylmethyl | H | CF₃ | H |
| Et | 2-chloro-thiazol-5-ylmethyl | H | CF₃ | H |
| Pr | 2-chloro-thiazol-5-ylmethyl | H | CF₃ | H |
| CH₂CF₃ | 2-chloro-thiazol-5-ylmethyl | H | CF₃ | H |
| CH₂C≡CH | 2-chloro-thiazol-5-ylmethyl | H | CF₃ | H |
| CycPr | 2-chloro-thiazol-5-ylmethyl | H | CF₃ | H |
| CH₂CycPr | 2-chloro-thiazol-5-ylmethyl | H | CF₃ | H |

TABLE 4

| R¹ | R⁶ | R⁸ | R⁹ | R¹⁰ |
|---|---|---|---|---|
| Me | Me | H | CF₂CF₃ | H |
| Et | Me | H | CF₂CF₃ | H |
| Pr | Me | H | CF₂CF₃ | H |
| CH₂CF₃ | Me | H | CF₂CF₃ | H |
| CH₂C≡CH | Me | H | CF₂CF₃ | H |
| CycPr | Me | H | CF₂CF₃ | H |
| CH₂CycPr | Me | H | CF₂CF₃ | H |
| Me | Et | H | CF₂CF₃ | H |
| Et | Et | H | CF₂CF₃ | H |
| Pr | Et | H | CF₂CF₃ | H |
| CH₂CF₃ | Et | H | CF₂CF₃ | H |
| CH₂C≡CH | Et | H | CF₂CF₃ | H |
| CycPr | Et | H | CF₂CF₃ | H |
| CH₂CycPr | Et | H | CF₂CF₃ | H |
| Me | CycPr | H | CF₂CF₃ | H |
| Et | CycPr | H | CF₂CF₃ | H |
| Pr | CycPr | H | CF₂CF₃ | H |
| CH₂CF₃ | CycPr | H | CF₂CF₃ | H |
| CH₂C≡CH | CycPr | H | CF₂CF₃ | H |
| CycPr | CycPr | H | CF₂CF₃ | H |
| CH₂CycPr | CycPr | H | CF₂CF₃ | H |

TABLE 5

| R¹ | R⁶ | R⁸ | R⁹ | R¹⁰ |
|---|---|---|---|---|
| Me | CH₂CycPr | H | CF₂CF₃ | H |
| Et | CH₂CycPr | H | CF₂CF₃ | H |
| Pr | CH₂CycPr | H | CF₂CF₃ | H |
| CH₂CF₃ | CH₂CycPr | H | CF₂CF₃ | H |
| CH₂C≡CH | CH₂CycPr | H | CF₂CF₃ | H |
| CycPr | CH₂CycPr | H | CF₂CF₃ | H |
| CH₂CycPr | CH₂CycPr | H | CF₂CF₃ | H |
| Me | CH₂C≡CH | H | CF₂CF₃ | H |
| Et | CH₂C≡CH | H | CF₂CF₃ | H |
| Pr | CH₂C≡CH | H | CF₂CF₃ | H |
| CH₂CF₃ | CH₂C≡CH | H | CF₂CF₃ | H |
| CH₂C≡CH | CH₂C≡CH | H | CF₂CF₃ | H |
| CycPr | CH₂C≡CH | H | CF₂CF₃ | H |
| CH₂CycPr | CH₂C≡CH | H | CF₂CF₃ | H |
| Me | CH₂OCH₃ | H | CF₂CF₃ | H |
| Et | CH₂OCH₃ | H | CF₂CF₃ | H |
| Pr | CH₂OCH₃ | H | CF₂CF₃ | H |
| CH₂CF₃ | CH₂OCH₃ | H | CF₂CF₃ | H |
| CH₂C≡CH | CH₂OCH₃ | H | CF₂CF₃ | H |
| CycPr | CH₂OCH₃ | H | CF₂CF₃ | H |
| CH₂CycPr | CH₂OCH₃ | H | CF₂CF₃ | H |

TABLE 6

| R¹ | R⁶ | R⁸ | R⁹ | R¹⁰ |
|---|---|---|---|---|
| Me | CO₂CH₃ | H | CF₂CF₃ | H |
| Et | CO₂CH₃ | H | CF₂CF₃ | H |
| Pr | CO₂CH₃ | H | CF₂CF₃ | H |
| CH₂CF₃ | CO₂CH₃ | H | CF₂CF₃ | H |
| CH₂C≡CH | CO₂CH₃ | H | CF₂CF₃ | H |
| CycPr | CO₂CH₃ | H | CF₂CF₃ | H |
| CH₂CycPr | CO₂CH₃ | H | CF₂CF₃ | H |
| Me | 6-chloro-pyridin-3-ylmethyl | H | CF₂CF₃ | H |
| Et | 6-chloro-pyridin-3-ylmethyl | H | CF₂CF₃ | H |
| Pr | 6-chloro-pyridin-3-ylmethyl | H | CF₂CF₃ | H |
| CH₂CF₃ | 6-chloro-pyridin-3-ylmethyl | H | CF₂CF₃ | H |
| CH₂C≡CH | 6-chloro-pyridin-3-ylmethyl | H | CF₂CF₃ | H |
| CycPr | 6-chloro-pyridin-3-ylmethyl | H | CF₂CF₃ | H |
| CH₂CycPr | 6-chloro-pyridin-3-ylmethyl | H | CF₂CF₃ | H |
| Me | 2-chloro-thiazol-5-ylmethyl | H | CF₂CF₃ | H |
| Et | 2-chloro-thiazol-5-ylmethyl | H | CF₂CF₃ | H |
| Pr | 2-chloro-thiazol-5-ylmethyl | H | CF₂CF₃ | H |
| CH₂CF₃ | 2-chloro-thiazol-5-ylmethyl | H | CF₂CF₃ | H |
| CH₂C≡CH | 2-chloro-thiazol-5-ylmethyl | H | CF₂CF₃ | H |
| CycPr | 2-chloro-thiazol-5-ylmethyl | H | CF₂CF₃ | H |
| CH₂CycPr | 2-chloro-thiazol-5-ylmethyl | H | CF₂CF₃ | H |

TABLE 7

| R¹ | R⁶ | R⁸ | R⁹ | R¹⁰ |
|---|---|---|---|---|
| Me | Me | H | CF(CF₃)₂ | H |
| Et | Me | H | CF(CF₃)₂ | H |
| Pr | Me | H | CF(CF₃)₂ | H |
| CH₂CF₃ | Me | H | CF(CF₃)₂ | H |
| CH₂C≡CH | Me | H | CF(CF₃)₂ | H |
| CycPr | Me | H | CF(CF₃)₂ | H |
| CH₂CycPr | Me | H | CF(CF₃)₂ | H |
| Me | Et | H | CF(CF₃)₂ | H |
| Et | Et | H | CF(CF₃)₂ | H |
| Pr | Et | H | CF(CF₃)₂ | H |
| CH₂CF₃ | Et | H | CF(CF₃)₂ | H |
| CH₂C≡CH | Et | H | CF(CF₃)₂ | H |
| CycPr | Et | H | CF(CF₃)₂ | H |
| CH₂CycPr | Et | H | CF(CF₃)₂ | H |
| Me | CycPr | H | CF(CF₃)₂ | H |
| Et | CycPr | H | CF(CF₃)₂ | H |
| Pr | CycPr | H | CF(CF₃)₂ | H |
| CH₂CF₃ | CycPr | H | CF(CF₃)₂ | H |
| CH₂C≡CH | CycPr | H | CF(CF₃)₂ | H |
| CycPr | CycPr | H | CF(CF₃)₂ | H |
| CH₂CycPr | CycPr | H | CF(CF₃)₂ | H |

TABLE 8

| R¹ | R⁶ | R⁸ | R⁹ | R¹⁰ |
|---|---|---|---|---|
| Me | CH₂CycPr | H | CF(CF₃)₂ | H |
| Et | CH₂CycPr | H | CF(CF₃)₂ | H |
| Pr | CH₂CycPr | H | CF(CF₃)₂ | H |
| CH₂CF₃ | CH₂CycPr | H | CF(CF₃)₂ | H |
| CH₂C≡CH | CH₂CycPr | H | CF(CF₃)₂ | H |
| CycPr | CH₂CycPr | H | CF(CF₃)₂ | H |
| CH₂CycPr | CH₂CycPr | H | CF(CF₃)₂ | H |
| Me | CH₂C≡CH | H | CF(CF₃)₂ | H |
| Et | CH₂C≡CH | H | CF(CF₃)₂ | H |
| Pr | CH₂C≡CH | H | CF(CF₃)₂ | H |
| CH₂CF₃ | CH₂C≡CH | H | CF(CF₃)₂ | H |
| CH₂C≡CH | CH₂C≡CH | H | CF(CF₃)₂ | H |
| CycPr | CH₂C≡CH | H | CF(CF₃)₂ | H |
| CH₂CycPr | CH₂C≡CH | H | CF(CF₃)₂ | H |
| Me | CH₂OCH₃ | H | CF(CF₃)₂ | H |
| Et | CH₂OCH₃ | H | CF(CF₃)₂ | H |
| Pr | CH₂OCH₃ | H | CF(CF₃)₂ | H |
| CH₂CF₃ | CH₂OCH₃ | H | CF(CF₃)₂ | H |
| CH₂C≡CH | CH₂OCH₃ | H | CF(CF₃)₂ | H |
| CycPr | CH₂OCH₃ | H | CF(CF₃)₂ | H |
| CH₂CycPr | CH₂OCH₃ | H | CF(CF₃)2 | H |

TABLE 9

| R¹ | R⁶ | R⁸ | R⁹ | R¹⁰ |
|---|---|---|---|---|
| Me | CO₂CH₃ | H | CF(CF₃)₂ | H |
| Et | CO₂CH₃ | H | CF(CF₃)₂ | H |
| Pr | CO₂CH₃ | H | CF(CF₃)₂ | H |
| CH₂CF₃ | CO₂CH₃ | H | CF(CF₃)₂ | H |
| CH₂C≡CH | CO₂CH₃ | H | CF(CF₃)₂ | H |
| CycPr | CO₂CH₃ | H | CF(CF₃)₂ | H |
| CH₂CycPr | CO₂CH₃ | H | CF(CF₃)₂ | H |
| Me | 6-chloro-pyridin-3-ylmethyl | H | CF(CF₃)₂ | H |
| Et | 6-chloro-pyridin-3-ylmethyl | H | CF(CF₃)₂ | H |
| Pr | 6-chloro-pyridin-3-ylmethyl | H | CF(CF₃)₂ | H |
| CH₂CF₃ | 6-chloro-pyridin-3-ylmethyl | H | CF(CF₃)₂ | H |
| CH₂C≡CH | 6-chloro-pyridin-3-ylmethyl | H | CF(CF₃)₂ | H |
| CycPr | 6-chloro-pyridin-3-ylmethyl | H | CF(CF₃)₂ | H |
| CH₂CycPr | 6-chloro-pyridin-3-ylmethyl | H | CF(CF₃)₂ | H |
| Me | 2-chloro-thiazol-5-ylmethyl | H | CF(CF₃)₂ | H |
| Et | 2-chloro-thiazol-5-ylmethyl | H | CF(CF₃)₂ | H |
| Pr | 2-chloro-thiazol-5-ylmethyl | H | CF(CF₃)₂ | H |
| CH₂CF₃ | 2-chloro-thiazol-5-ylmethyl | H | CF(CF₃)₂ | H |
| CH₂C≡CH | 2-chloro-thiazol-5-ylmethyl | H | CF(CF₃)₂ | H |
| CycPr | 2-chloro-thiazol-5-ylmethyl | H | CF(CF₃)₂ | H |
| CH₂CycPr | 2-chloro-thiazol-5-ylmethyl | H | CF(CF₃)₂ | H |

TABLE 10

| R¹ | R⁶ | R⁸ | R⁹ | R¹⁰ |
|---|---|---|---|---|
| Me | Me | H | OCF₃ | H |
| Et | Me | H | OCF₃ | H |
| Pr | Me | H | OCF₃ | H |
| CH₂CF₃ | Me | H | OCF₃ | H |
| CH₂C≡CH | Me | H | OCF₃ | H |
| CycPr | Me | H | OCF₃ | H |
| CH₂CycPr | Me | H | OCF₃ | H |
| Me | Et | H | OCF₃ | H |
| Et | Et | H | OCF₃ | H |
| Pr | Et | H | OCF₃ | H |
| CH₂CF₃ | Et | H | OCF₃ | H |
| CH₂C≡CH | Et | H | OCF₃ | H |
| CycPr | Et | H | OCF₃ | H |
| CH₂CycPr | Et | H | OCF₃ | H |
| Me | CycPr | H | OCF₃ | H |
| Et | CycPr | H | OCF₃ | H |
| Pr | CycPr | H | OCF₃ | H |
| CH₂CF₃ | CycPr | H | OCF₃ | H |
| CH₂C≡CH | CycPr | H | OCF₃ | H |
| CycPr | CycPr | H | OCF₃ | H |
| CH₂CycPr | CycPr | H | OCF₃ | H |

TABLE 11

| R¹ | R⁶ | R⁸ | R⁹ | R¹⁰ |
|---|---|---|---|---|
| Me | CH₂CycPr | H | OCF₃ | H |
| Et | CH₂CycPr | H | OCF₃ | H |
| Pr | CH₂CycPr | H | OCF₃ | H |
| CH₂CF₃ | CH₂CycPr | H | OCF₃ | H |
| CH₂C≡CH | CH₂CycPr | H | OCF₃ | H |
| CycPr | CH₂CycPr | H | OCF₃ | H |
| CH₂CycPr | CH₂CycPr | H | OCF₃ | H |
| Me | CH₂C≡CH | H | OCF₃ | H |
| Et | CH₂C≡CH | H | OCF₃ | H |
| Pr | CH₂C≡CH | H | OCF₃ | H |
| CH₂CF₃ | CH₂C≡CH | H | OCF₃ | H |
| CH₂C≡CH | CH₂C≡CH | H | OCF₃ | H |
| CycPr | CH₂C≡CH | H | OCF₃ | H |
| CH₂CycPr | CH₂C≡CH | H | OCF₃ | H |
| Me | CH₂OCH₃ | H | OCF₃ | H |
| Et | CH₂OCH₃ | H | OCF₃ | H |
| Pr | CH₂OCH₃ | H | OCF₃ | H |
| CH₂CF₃ | CH₂OCH₃ | H | OCF₃ | H |
| CH₂C≡CH | CH₂OCH₃ | H | OCF₃ | H |
| CycPr | CH₂OCH₃ | H | OCF₃ | H |
| CH₂CycPr | CH₂OCH₃ | H | OCF₃ | H |

TABLE 12

| R¹ | R⁶ | R⁸ | R⁹ | R¹⁰ |
|---|---|---|---|---|
| Me | CO₂CH₃ | H | OCF₃ | H |
| Et | CO₂CH₃ | H | OCF₃ | H |
| Pr | CO₂CH₃ | H | OCF₃ | H |
| CH₂CF₃ | CO₂CH₃ | H | OCF₃ | H |
| CH₂C≡CH | CO₂CH₃ | H | OCF₃ | H |
| CycPr | CO₂CH₃ | H | OCF₃ | H |
| CH₂CycPr | CO₂CH₃ | H | OCF₃ | H |
| Me | 6-chloro-pyridin-3-ylmethyl | H | OCF₃ | H |
| Et | 6-chloro-pyridin-3-ylmethyl | H | OCF₃ | H |
| Pr | 6-chloro-pyridin-3-ylmethyl | H | OCF₃ | H |
| CH₂CF₃ | 6-chloro-pyridin-3-ylmethyl | H | OCF₃ | H |
| CH₂C≡CH | 6-chloro-pyridin-3-ylmethyl | H | OCF₃ | H |
| CycPr | 6-chloro-pyridin-3-ylmethyl | H | OCF₃ | H |
| CH₂CycPr | 6-chloro-pyridin-3-ylmethyl | H | OCF₃ | H |
| Me | 2-chloro-thiazol-5-ylmethyl | H | OCF₃ | H |
| Et | 2-chloro-thiazol-5-ylmethyl | H | OCF₃ | H |
| Pr | 2-chloro-thiazol-5-ylmethyl | H | OCF₃ | H |
| CH₂CF₃ | 2-chloro-thiazol-5-ylmethyl | H | OCF₃ | H |
| CH₂C≡CH | 2-chloro-thiazol-5-ylmethyl | H | OCF₃ | H |
| CycPr | 2-chloro-thiazol-5-ylmethyl | H | OCF₃ | H |
| CH₂CycPr | 2-chloro-thiazol-5-ylmethyl | H | OCF₃ | H |

TABLE 13

| R¹ | R⁶ | R⁸ | R⁹ | R¹⁰ |
|---|---|---|---|---|
| Me | Me | H | SCF₃ | H |
| Et | Me | H | SCF₃ | H |
| Pr | Me | H | SCF₃ | H |
| CH₂CF₃ | Me | H | SCF₃ | H |
| CH₂C≡CH | Me | H | SCF₃ | H |
| CycPr | Me | H | SCF₃ | H |
| CH₂CycPr | Me | H | SCF₃ | H |
| Me | Et | H | SCF₃ | H |
| Et | Et | H | SCF₃ | H |
| Pr | Et | H | SCF₃ | H |
| CH₂CF₃ | Et | H | SCF₃ | H |
| CH₂C≡CH | Et | H | SCF₃ | H |
| CycPr | Et | H | SCF₃ | H |
| CH₂CycPr | Et | H | SCF₃ | H |
| Me | CycPr | H | SCF₃ | H |
| Et | CycPr | H | SCF₃ | H |
| Pr | CycPr | H | SCF₃ | H |
| CH₂CF₃ | CycPr | H | SCF₃ | H |
| CH₂C≡CH | CycPr | H | SCF₃ | H |
| CycPr | CycPr | H | SCF₃ | H |
| CH₂CycPr | CycPr | H | SCF₃ | H |

TABLE 14

| R¹ | R⁶ | R⁸ | R⁹ | R¹⁰ |
|---|---|---|---|---|
| Me | CH₂CycPr | H | SCF₃ | H |
| Et | CH₂CycPr | H | SCF₃ | H |
| Pr | CH₂CycPr | H | SCF₃ | H |
| CH₂CF₃ | CH₂CycPr | H | SCF₃ | H |
| CH₂C≡CH | CH₂CycPr | H | SCF₃ | H |
| CycPr | CH₂CycPr | H | SCF₃ | H |
| CH₂CycPr | CH₂CycPr | H | SCF₃ | H |
| Me | CH₂C≡CH | H | SCF₃ | H |
| Et | CH₂C≡CH | H | SCF₃ | H |
| Pr | CH₂C≡CH | H | SCF₃ | H |
| CH₂CF₃ | CH₂C≡CH | H | SCF₃ | H |
| CH₂C≡CH | CH₂C≡CH | H | SCF₃ | H |
| CycPr | CH₂C≡CH | H | SCF₃ | H |
| CH₂CycPr | CH₂C≡CH | H | SCF₃ | H |
| Me | CH₂OCH₃ | H | SCF₃ | H |
| Et | CH₂OCH₃ | H | SCF₃ | H |
| Pr | CH₂OCH₃ | H | SCF₃ | H |
| CH₂CF₃ | CH₂OCH₃ | H | SCF₃ | H |
| CH₂C≡CH | CH₂OCH₃ | H | SCF₃ | H |
| CycPr | CH₂OCH₃ | H | SCF₃ | H |
| CH₂CycPr | CH₂OCH₃ | H | SCF₃ | H |

TABLE 15

| R¹ | R⁶ | R⁸ | R⁹ | R¹⁰ |
|---|---|---|---|---|
| Me | CO₂CH₃ | H | SCF₃ | H |
| Et | CO₂CH₃ | H | SCF₃ | H |
| Pr | CO₂CH₃ | H | SCF₃ | H |
| CH₂CF₃ | CO₂CH₃ | H | SCF₃ | H |
| CH₂C≡CH | CO₂CH₃ | H | SCF₃ | H |
| CycPr | CO₂CH₃ | H | SCF₃ | H |
| CH₂CycPr | CO₂CH₃ | H | SCF₃ | H |
| Me | 6-chloro-pyridin-3-ylmethyl | H | SCF₃ | H |
| Et | 6-chloro-pyridin-3-ylmethyl | H | SCF₃ | H |
| Pr | 6-chloro-pyridin-3-ylmethyl | H | SCF₃ | H |
| CH₂CF₃ | 6-chloro-pyridin-3-ylmethyl | H | SCF₃ | H |
| CH₂C≡CH | 6-chloro-pyridin-3-ylmethyl | H | SCF₃ | H |
| CycPr | 6-chloro-pyridin-3-ylmethyl | H | SCF₃ | H |
| CH₂CycPr | 6-chloro-pyridin-3-ylmethyl | H | SCF₃ | H |
| Me | 2-chloro-thiazol-5-ylmethyl | H | SCF₃ | H |
| Et | 2-chloro-thiazol-5-ylmethyl | H | SCF₃ | H |
| Pr | 2-chloro-thiazol-5-ylmethyl | H | SCF₃ | H |
| CH₂CF₃ | 2-chloro-thiazol-5-ylmethyl | H | SCF₃ | H |
| CH₂C≡CH | 2-chloro-thiazol-5-ylmethyl | H | SCF₃ | H |
| CycPr | 2-chloro-thiazol-5-ylmethyl | H | SCF₃ | H |
| CH₂CycPr | 2-chloro-thiazol-5-ylmethyl | H | SCF₃ | H |

TABLE 16

| R¹ | R⁶ | R⁸ | R⁹ | R¹⁰ |
|---|---|---|---|---|
| Me | Me | H | S(O)CF₃ | H |
| Et | Me | H | S(O)CF₃ | H |
| Pr | Me | H | S(O)CF₃ | H |
| CH₂CF₃ | Me | H | S(O)CF₃ | H |
| CH₂C≡CH | Me | H | S(O)CF₃ | H |
| CycPr | Me | H | S(O)CF₃ | H |
| CH₂CycPr | Me | H | S(O)CF₃ | H |
| Me | Et | H | S(O)CF₃ | H |
| Et | Et | H | S(O)CF₃ | H |
| Pr | Et | H | S(O)CF₃ | H |
| CH₂CF₃ | Et | H | S(O)CF₃ | H |
| CH₂C≡CH | Et | H | S(O)CF₃ | H |
| CycPr | Et | H | S(O)CF₃ | H |
| CH₂CycPr | Et | H | S(O)CF₃ | H |
| Me | CycPr | H | S(O)CF₃ | H |
| Et | CycPr | H | S(O)CF₃ | H |
| Pr | CycPr | H | S(O)CF₃ | H |
| CH₂CF₃ | CycPr | H | S(O)CF₃ | H |
| CH₂C≡CH | CycPr | H | S(O)CF₃ | H |
| CycPr | CycPr | H | S(O)CF₃ | H |
| CH₂CycPr | CycPr | H | S(O)CF₃ | H |

TABLE 17

| R¹ | R⁶ | R⁸ | R⁹ | R¹⁰ |
|---|---|---|---|---|
| Me | CH₂CycPr | H | S(O)CF₃ | H |
| Et | CH₂CycPr | H | S(O)CF₃ | H |
| Pr | CH₂CycPr | H | S(O)CF₃ | H |
| CH₂CF₃ | CH₂CycPr | H | S(O)CF₃ | H |
| CH₂C≡CH | CH₂CycPr | H | S(O)CF₃ | H |
| CycPr | CH₂CycPr | H | S(O)CF₃ | H |
| CH₂CycPr | CH₂CycPr | H | S(O)CF₃ | H |
| Me | CH₂C≡CH | H | S(O)CF₃ | H |
| Et | CH₂C≡CH | H | S(O)CF₃ | H |
| Pr | CH₂C≡CH | H | S(O)CF₃ | H |
| CH₂CF₃ | CH₂C≡CH | H | S(O)CF₃ | H |
| CH₂C≡CH | CH₂C≡CH | H | S(O)CF₃ | H |
| CycPr | CH₂C≡CH | H | S(O)CF₃ | H |
| CH₂CycPr | CH₂C≡CH | H | S(O)CF₃ | H |
| Me | CH₂OCH₃ | H | S(O)CF₃ | H |
| Et | CH₂OCH₃ | H | S(O)CF₃ | H |
| Pr | CH₂OCH₃ | H | S(O)CF₃ | H |
| CH₂CF₃ | CH₂OCH₃ | H | S(O)CF₃ | H |
| CH₂C≡CH | CH₂OCH₃ | H | S(O)CF₃ | H |
| CycPr | CH₂OCH₃ | H | S(O)CF₃ | H |
| CH₂CycPr | CH₂OCH₃ | H | S(O)CF₃ | H |

TABLE 18

| R¹ | R⁶ | R⁸ | R⁹ | R¹⁰ |
|---|---|---|---|---|
| Me | CO₂CH₃ | H | S(O)CF₃ | H |
| Et | CO₂CH₃ | H | S(O)CF₃ | H |
| Pr | CO₂CH₃ | H | S(O)CF₃ | H |
| CH₂CF₃ | CO₂CH₃ | H | S(O)CF₃ | H |
| CH₂C≡CH | CO₂CH₃ | H | S(O)CF₃ | H |
| CycPr | CO₂CH₃ | H | S(O)CF₃ | H |
| CH₂CycPr | CO₂CH₃ | H | S(O)CF₃ | H |
| Me | 6-chloro-pyridin-3-ylmethyl | H | S(O)CF₃ | H |
| Et | 6-chloro-pyridin-3-ylmethyl | H | S(O)CF₃ | H |
| Pr | 6-chloro-pyridin-3-ylmethyl | H | S(O)CF₃ | H |
| CH₂CF₃ | 6-chloro-pyridin-3-ylmethyl | H | S(O)CF₃ | H |
| CH₂C≡CH | 6-chloro-pyridin-3-ylmethyl | H | S(O)CF₃ | H |
| CycPr | 6-chloro-pyridin-3-ylmethyl | H | S(O)CF₃ | H |
| CH₂CycPr | 6-chloro-pyridin-3-ylmethyl | H | S(O)CF₃ | H |
| Me | 2-chloro-thiazol-5-ylmethyl | H | S(O)CF₃ | H |
| Et | 2-chloro-thiazol-5-ylmethyl | H | S(O)CF₃ | H |
| Pr | 2-chloro-thiazol-5-ylmethyl | H | S(O)CF₃ | H |
| CH₂CF₃ | 2-chloro-thiazol-5-ylmethyl | H | S(O)CF₃ | H |
| CH₂C≡CH | 2-chloro-thiazol-5-ylmethyl | H | S(O)CF₃ | H |
| CycPr | 2-chloro-thiazol-5-ylmethyl | H | S(O)CF₃ | H |
| CH₂CycPr | 2-chloro-thiazol-5-ylmethyl | H | S(O)CF₃ | H |

TABLE 19

| R¹ | R⁶ | R⁸ | R⁹ | R¹⁰ |
|---|---|---|---|---|
| Me | Me | H | S(O)₂CF₃ | H |
| Et | Me | H | S(O)₂CF₃ | H |
| Pr | Me | H | S(O)₂CF₃ | H |
| CH₂CF₃ | Me | H | S(O)₂CF₃ | H |
| CH₂C≡CH | Me | H | S(O)₂CF₃ | H |
| CycPr | Me | H | S(O)₂CF₃ | H |
| CH₂CycPr | Me | H | S(O)₂CF₃ | H |
| Me | Et | H | S(O)₂CF₃ | H |
| Et | Et | H | S(O)₂CF₃ | H |
| Pr | Et | H | S(O)₂CF₃ | H |
| CH₂CF₃ | Et | H | S(O)₂CF₃ | H |
| CH₂C≡CH | Et | H | S(O)₂CF₃ | H |
| CycPr | Et | H | S(O)₂CF₃ | H |
| CH₂CycPr | Et | H | S(O)₂CF₃ | H |
| Me | CycPr | H | S(O)₂CF₃ | H |
| Et | CycPr | H | S(O)₂CF₃ | H |
| Pr | CycPr | H | S(O)₂CF₃ | H |
| CH₂CF₃ | CycPr | H | S(O)₂CF₃ | H |
| CH₂C≡CH | CycPr | H | S(O)₂CF₃ | H |
| CycPr | CycPr | H | S(O)₂CF₃ | H |
| CH₂CycPr | CycPr | H | S(O)₂CF₃ | H |

TABLE 20

| R¹ | R⁶ | R⁸ | R⁹ | R¹⁰ |
|---|---|---|---|---|
| Me | CH₂CycPr | H | S(O)₂CF₃ | H |
| Et | CH₂CycPr | H | S(O)₂CF₃ | H |
| Pr | CH₂CycPr | H | S(O)₂CF₃ | H |
| CH₂CF₃ | CH₂CycPr | H | S(O)₂CF₃ | H |
| CH₂C≡CH | CH₂CycPr | H | S(O)₂CF₃ | H |
| CycPr | CH₂CycPr | H | S(O)₂CF₃ | H |
| CH₂CycPr | CH₂CycPr | H | S(O)₂CF₃ | H |
| Me | CH₂C≡CH | H | S(O)₂CF₃ | H |
| Et | CH₂C≡CH | H | S(O)₂CF₃ | H |
| Pr | CH₂C≡CH | H | S(O)₂CF₃ | H |
| CH₂CF₃ | CH₂C≡CH | H | S(O)₂CF₃ | H |
| CH₂C≡CH | CH₂C≡CH | H | S(O)₂CF₃ | H |
| CycPr | CH₂C≡CH | H | S(O)₂CF₃ | H |
| CH₂CycPr | CH₂C≡CH | H | S(O)₂CF₃ | H |
| Me | CH₂OCH₃ | H | S(O)₂CF₃ | H |
| Et | CH₂OCH₃ | H | S(O)₂CF₃ | H |
| Pr | CH₂OCH₃ | H | S(O)₂CF₃ | H |
| CH₂CF₃ | CH₂OCH₃ | H | S(O)₂CF₃ | H |
| CH₂C≡CH | CH₂OCH₃ | H | S(O)₂CF₃ | H |
| CycPr | CH₂OCH₃ | H | S(O)₂CF₃ | H |
| CH₂CycPr | CH₂OCH₃ | H | S(O)₂CF₃ | H |

TABLE 21

| R¹ | R⁶ | R⁸ | R⁹ | R¹⁰ |
|---|---|---|---|---|
| Me | CO₂CH₃ | H | S(O)₂CF₃ | H |
| Et | CO₂CH₃ | H | S(O)₂CF₃ | H |
| Pr | CO₂CH₃ | H | S(O)₂CF₃ | H |
| CH₂CF₃ | CO₂CH₃ | H | S(O)₂CF₃ | H |
| CH₂C≡CH | CO₂CH₃ | H | S(O)₂CF₃ | H |
| CycPr | CO₂CH₃ | H | S(O)₂CF₃ | H |
| CH₂CycPr | CO₂CH₃ | H | S(O)₂CF₃ | H |
| Me | 6-chloro-pyridin-3-ylmethyl | H | S(O)₂CF₃ | H |
| Et | 6-chloro-pyridin-3-ylmethyl | H | S(O)₂CF₃ | H |
| Pr | 6-chloro-pyridin-3-ylmethyl | H | S(O)₂CF₃ | H |
| CH₂CF₃ | 6-chloro-pyridin-3-ylmethyl | H | S(O)₂CF₃ | H |
| CH₂C≡CH | 6-chloro-pyridin-3-ylmethyl | H | S(O)₂CF₃ | H |
| CycPr | 6-chloro-pyridin-3-ylmethyl | H | S(O)₂CF₃ | H |
| CH₂CycPr | 6-chloro-pyridin-3-ylmethyl | H | S(O)₂CF₃ | H |
| Me | 2-chloro-thiazol-5-ylmethyl | H | S(O)₂CF₃ | H |
| Et | 2-chloro-thiazol-5-ylmethyl | H | S(O)₂CF₃ | H |
| Pr | 2-chloro-thiazol-5-ylmethyl | H | S(O)₂CF₃ | H |
| CH₂CF₃ | 2-chloro-thiazol-5-ylmethyl | H | S(O)₂CF₃ | H |
| CH₂C≡CH | 2-chloro-thiazol-5-ylmethyl | H | S(O)₂CF₃ | H |
| CycPr | 2-chloro-thiazol-5-ylmethyl | H | S(O)₂CF₃ | H |
| CH₂CycPr | 2-chloro-thiazol-5-ylmethyl | H | S(O)₂CF₃ | H |

TABLE 22

| R¹ | R⁶ | R⁸ | R⁹ | R¹⁰ |
|---|---|---|---|---|
| Me | Me | CF₃ | H | CF₃ |
| Et | Me | CF₃ | H | CF₃ |
| Pr | Me | CF₃ | H | CF₃ |
| CH₂CF₃ | Me | CF₃ | H | CF₃ |
| CH₂C≡CH | Me | CF₃ | H | CF₃ |
| CycPr | Me | CF₃ | H | CF₃ |
| CH₂CycPr | Me | CF₃ | H | CF₃ |
| Me | Et | CF₃ | H | CF₃ |
| Et | Et | CF₃ | H | CF₃ |
| Pr | Et | CF₃ | H | CF₃ |
| CH₂CF₃ | Et | CF₃ | H | CF₃ |
| CH₂C≡CH | Et | CF₃ | H | CF₃ |
| CycPr | Et | CF₃ | H | CF₃ |
| CH₂CycPr | Et | CF₃ | H | CF₃ |
| Me | CycPr | CF₃ | H | CF₃ |
| Et | CycPr | CF₃ | H | CF₃ |
| Pr | CycPr | CF₃ | H | CF₃ |
| CH₂CF₃ | CycPr | CF₃ | H | CF₃ |
| CH₂C≡CH | CycPr | CF₃ | H | CF₃ |
| CycPr | CycPr | CF₃ | H | CF₃ |
| CH₂CycPr | CycPr | CF₃ | H | CF₃ |

TABLE 23

| R¹ | R⁶ | R⁸ | R⁹ | R¹⁰ |
|---|---|---|---|---|
| Me | CH₂CycPr | CF₃ | H | CF₃ |
| Et | CH₂CycPr | CF₃ | H | CF₃ |
| Pr | CH₂CycPr | CF₃ | H | CF₃ |
| CH₂CF₃ | CH₂CycPr | CF₃ | H | CF₃ |
| CH₂C≡CH | CH₂CycPr | CF₃ | H | CF₃ |
| CycPr | CH₂CycPr | CF₃ | H | CF₃ |
| CH₂CycPr | CH₂CycPr | CF₃ | H | CF₃ |
| Me | CH₂C≡CH | CF₃ | H | CF₃ |
| Et | CH₂C≡CH | CF₃ | H | CF₃ |
| Pr | CH₂C≡CH | CF₃ | H | CF₃ |
| CH₂CF₃ | CH₂C≡CH | CF₃ | H | CF₃ |
| CH₂C≡CH | CH₂C≡CH | CF₃ | H | CF₃ |
| CycPr | CH₂C≡CH | CF₃ | H | CF₃ |
| CH₂CycPr | CH₂C≡CH | CF₃ | H | CF₃ |
| Me | CH₂OCH₃ | CF₃ | H | CF₃ |
| Et | CH₂OCH₃ | CF₃ | H | CF₃ |
| Pr | CH₂OCH₃ | CF₃ | H | CF₃ |
| CH₂CF₃ | CH₂OCH₃ | CF₃ | H | CF₃ |
| CH₂C≡CH | CH₂OCH₃ | CF₃ | H | CF₃ |
| CycPr | CH₂OCH₃ | CF₃ | H | CF₃ |
| CH₂CycPr | CH₂OCH₃ | CF₃ | H | CF₃ |

TABLE 24

| R¹ | R⁶ | R⁸ | R⁹ | R¹⁰ |
|---|---|---|---|---|
| Me | CO₂CH₃ | CF₃ | H | CF₃ |
| Et | CO₂CH₃ | CF₃ | H | CF₃ |
| Pr | CO₂CH₃ | CF₃ | H | CF₃ |
| CH₂CF₃ | CO₂CH₃ | CF₃ | H | CF₃ |
| CH₂C≡CH | CO₂CH₃ | CF₃ | H | CF₃ |
| CycPr | CO₂CH₃ | CF₃ | H | CF₃ |
| CH₂CycPr | CO₂CH₃ | CF₃ | H | CF₃ |
| Me | 6-chloro-pyridin-3-ylmethyl | CF₃ | H | CF₃ |
| Et | 6-chloro-pyridin-3-ylmethyl | CF₃ | H | CF₃ |
| Pr | 6-chloro-pyridin-3-ylmethyl | CF₃ | H | CF₃ |
| CH₂CF₃ | 6-chloro-pyridin-3-ylmethyl | CF₃ | H | CF₃ |
| CH₂C≡CH | 6-chloro-pyridin-3-ylmethyl | CF₃ | H | CF₃ |
| CycPr | 6-chloro-pyridin-3-ylmethyl | CF₃ | H | CF₃ |
| CH₂CycPr | 6-chloro-pyridin-3-ylmethyl | CF₃ | H | CF₃ |
| Me | 2-chloro-thiazol-5-ylmethyl | CF₃ | H | CF₃ |
| Et | 2-chloro-thiazol-5-ylmethyl | CF₃ | H | CF₃ |
| Pr | 2-chloro-thiazol-5-ylmethyl | CF₃ | H | CF₃ |
| CH₂CF₃ | 2-chloro-thiazol-5-ylmethyl | CF₃ | H | CF₃ |
| CH₂C≡CH | 2-chloro-thiazol-5-ylmethyl | CF₃ | H | CF₃ |
| CycPr | 2-chloro-thiazol-5-ylmethyl | CF₃ | H | CF₃ |
| CH₂CycPr | 2-chloro-thiazol-5-ylmethyl | CF₃ | H | CF₃ |

(In [Table 1] to [Table 24] above, Me represents a methyl group, Et represents an ethyl group, Pr represents a n-propyl group, and CycPr represents a cyclopropyl group.)

In the formula (1), compounds of the present invention wherein n is 1, R², R³, R⁴, R⁵, R⁷ and R¹¹ are a hydrogen atom, Q is an oxygen atom, and R¹, R⁶, R⁹ and R¹⁰ are the combinations shown in [Table 1] to [Table 24].

In the formula (1), compounds of the present invention wherein n is 2, R², R³, R⁴, R⁵, R⁷ and R¹¹ are a hydrogen atom, Q is an oxygen atom, and R¹, R⁶, R⁸, R⁹ and R¹⁰ are the combinations shown in [Table 1] to [Table 24].

In the formula (1), compounds of the present invention wherein n is 0, R², R³, R⁴, R⁵, R⁷ and R¹¹ are a hydrogen atom, Q is a sulfur atom, and R¹, R⁶, R⁸, R⁹ and R¹⁰ are the combinations shown in [Table 1] to [Table 24].

In the formula (1), compounds of the present invention wherein n is 1, R², R³, R⁴, R⁵, R⁷ and R¹¹ are a hydrogen atom, Q is a sulfur atom, and R¹, R⁶, R⁸, R⁹ and R¹⁰ are the combinations shown in [Table 1] to [Table 24].

In the formula (1), compounds of the present invention wherein n is 2, R², R³, R⁴, R⁵, R⁷ and R¹¹ are a hydrogen atom, Q is a sulfur atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a fluorine atom, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is a fluorine atom, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a fluorine atom, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a chlorine atom, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is a chlorine atom, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a chlorine atom, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a bromine atom, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is a bromine atom, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a bromine atom, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a methyl group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is a methyl group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a methyl group, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a trifluoromethyl group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁹, R⁵, R⁷ and are a hydrogen atom, R³ is a trifluoromethyl group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a trifluoromethyl group, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a trifluoromethoxy group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is a trifluoromethoxy group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a trifluoromethoxy group, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a 2-pyridyl group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is a 2-pyridyl group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a 2-pyridyl group, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a 5-trifluoromethyl-2-pyridyl group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁴ and R¹¹ are a hydrogen atom, R³ is a 5-trifluoromethyl-2-pyridyl group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a 5-trifluoromethyl-2-pyridyl group, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a 2-pyrimidinyl group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is a 2-pyrimidinyl group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a 2-pyrimidinyl group, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a methoxycarbonyl group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is a methoxycarbonyl group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a methoxycarbonyl group, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is an ethoxycarbonyl group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is an ethoxycarbonyl group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is an ethoxycarbonyl group, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is an aminocarbonyl group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is an aminocarbonyl group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is an aminocarbonyl group, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, In the formula (1), n is 0, R², R⁴, R⁵, R⁷ and R¹¹ are a hydrogen atom, R³ is an amino group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is an amino group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is an amino group, Q is an oxygen atom, and R¹, R⁶, R⁸, R⁹ and R¹⁰ are the combinations shown in [Table 1] to [Table 24]. Compounds of the present invention wherein R³ is a methoxycarbonylamino group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is a methoxycarbonylamino group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a methoxycarbonylamino group, Q is an oxygen atom, and R¹, R⁶, R⁸, R⁹ and R¹⁰ are the combinations shown in [Table 1] to [Table 24].

In the formula (1), compounds of the present invention wherein n is 0, R², R⁴, R⁵, R⁷ and R¹¹ are a hydrogen atom, R³ is a tert-butoxycarbonylamino group, Q is an oxygen atom, and R¹, R⁶, R⁸, R⁹ and R¹⁰ are the combinations shown in [Table 1] to [Table 24].

In the formula (1), compounds of the present invention wherein n is 1, R², R⁴, R⁵, R⁷ and R¹¹ are a hydrogen atom, R³ is a tert-butoxycarbonylamino group, Q is an oxygen atom, and R¹, R⁶, R⁸, R⁹ and R¹⁰ are the combinations shown in [Table 1] to [Table 24].

In the formula (1), compounds of the present invention wherein n is 2, R², R⁴, R⁵, R⁷ and R¹¹ are a hydrogen atom, R³ is a tert-butoxycarbonylamino group, Q is an oxygen atom, and R¹, R⁶, R⁸, R⁹ and R¹⁰ are the combinations shown in [Table 1] to [Table 24].

Next, specific examples of the intermediate compound (M6) are shown below.

In the formula (M6), n is 0, R², R³, R⁴, R⁵, R⁷ and R¹¹ are a hydrogen atom, Q is an oxygen atom, and R¹, R⁸, R⁹ and R¹⁰ are the combinations shown in [Table 25] to [Table 27].

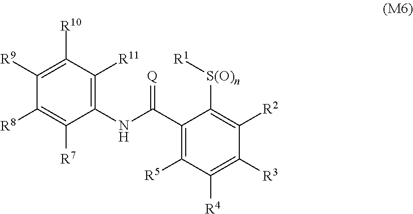

TABLE 25

| R¹ | R⁸ | R⁹ | R¹⁰ |
|---|---|---|---|
| Me | H | CF₃ | H |
| Et | H | CF₃ | H |
| Pr | H | CF₃ | H |
| CH₂CF₃ | H | CF₃ | H |
| CH₂C≡CH | H | CF₃ | H |
| CycPr | H | CF₃ | H |
| CH₂CycPr | H | CF₃ | H |
| Me | H | CF₂CF₃ | H |
| Et | H | CF₂CF₃ | H |
| Pr | H | CF₂CF₃ | H |
| CH₂CF₃ | H | CF₂CF₃ | H |
| CH₂C≡CH | H | CF₂CF₃ | H |
| CycPr | H | CF₂CF₃ | H |
| CH₂CycPr | H | CF₂CF₃ | H |
| Me | H | CF(CF₃)₂ | H |
| Et | H | CF(CF₃)₂ | H |
| Pr | H | CF(CF₃)₂ | H |
| CH₂CF₃ | H | CF(CF₃)₂ | H |
| CH₂C≡CH | H | CF(CF₃)₂ | H |
| CycPr | H | CF(CF₃)₂ | H |
| CH₂CycPr | H | CF(CF₃)₂ | H |

TABLE 26

| R¹ | R⁸ | R⁹ | R¹⁰ |
|---|---|---|---|
| Me | H | OCF₃ | H |
| Et | H | OCF₃ | H |
| Pr | H | OCF₃ | H |
| CH₂CF₃ | H | OCF₃ | H |
| CH₂C≡CH | H | OCF₃ | H |
| CycPr | H | OCF₃ | H |
| CH₂CycPr | H | OCF₃ | H |
| Me | H | SCF₃ | H |
| Et | H | SCF₃ | H |
| Pr | H | SCF₃ | H |
| CH₂CF₃ | H | SCF₃ | H |
| CH₂C≡CH | H | SCF₃ | H |
| CycPr | H | SCF₃ | H |
| CH₂CycPr | H | SCF₃ | H |
| Me | H | S(O)CF₃ | H |
| Et | H | S(O)CF₃ | H |
| Pr | H | S(O)CF₃ | H |
| CH₂CF₃ | H | S(O)CF₃ | H |
| CH₂C≡CH | H | S(O)CF₃ | H |
| CycPr | H | S(O)CF₃ | H |
| CH₂CycPr | H | S(O)CF₃ | H |

TABLE 27

| R¹ | R⁸ | R⁹ | R¹⁰ |
|---|---|---|---|
| Me | H | S(O)₂CF₃ | H |
| Et | H | S(O)₂CF₃ | H |
| Pr | H | S(O)₂CF₃ | H |
| CH₂CF₃ | H | S(O)₂CF₃ | H |
| CH₂C≡CH | H | S(O)₂CF₃ | H |
| CycPr | H | S(O)₂CF₃ | H |
| CH₂CycPr | H | S(O)₂CF₃ | H |
| Me | CF₃ | H | CF₃ |
| Et | CF₃ | H | CF₃ |
| Pr | CF₃ | H | CF₃ |
| CH₂CF₃ | CF₃ | H | CF₃ |
| CH₂C≡CH | CF₃ | H | CF₃ |
| CycPr | CF₃ | H | CF₃ |
| CH₂CycPr | CF₃ | H | CF₃ |

(In [Table 25] to [Table 27] above, Me represents a methyl group, Et represents an ethyl group, Pr represents a n-propyl group, and CycPr represents a cyclopropyl group.)
In the formula (M6), compounds of the present invention wherein n is 1, R², R³, R⁴, R⁵, R⁷ and R¹¹ are a hydrogen atom, Q is an oxygen atom, and R¹, R⁸, R⁹ and R¹⁰ are the combinations shown in [Table 25] to [Table 27].
In the formula (M6), compounds of the present invention wherein n is 2, R², R³, R⁴, R⁵, R⁷ and R¹¹ are a hydrogen atom, Q is an oxygen atom, and R¹, R⁸, R⁹ and R¹⁰ are the combinations shown in [Table 25] to [Table 27].

In the formula (M6), compounds of the present invention wherein n is 0, R², R³, R⁴, R⁵, R⁷ and R¹¹ are a hydrogen atom, Q is a sulfur atom, and R¹, R⁸, R⁹ and R¹⁰ are the combinations shown in [Table 25] to [Table 27].

In the formula (M6), compounds of the present invention wherein n is 1, R², R³, R⁴, R⁵, R⁷ and R¹¹ are a hydrogen atom, Q is a sulfur atom, and R¹, R⁸, R⁹ and R¹⁰ are the combinations shown in [Table 25] to [Table 27].
In the formula (M6), compounds of the present invention wherein n is 2, R², R³, R⁴, R⁵, R⁷ and R¹¹ are a hydrogen atom, Q is a sulfur atom, and R¹, R⁸, R⁹ and R¹⁰ are the combinations shown in [Table 25] to [Table 27].

In the formula (M6), compounds of the present invention wherein n is 0, R², R⁴, R⁵, R⁷ and R¹¹ are a hydrogen atom, R³ is a fluorine atom, Q is an oxygen atom, and R¹, R⁸, R⁹ and R¹⁰ are the combinations shown in [Table 25] to [Table 27].

In the formula (M6), compounds of the present invention wherein n is 1, R², R⁴, R⁵, R⁷ and R¹¹ are a hydrogen atom, R³ is a fluorine atom, Q is an oxygen atom, and R¹, R⁸, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is a fluorine atom, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is a chlorine atom, Q is an oxygen atom, and R¹, R⁸, R⁹ and R¹⁰ are the combinations shown in [Table 25] to [Table 27].

In the formula (M6), compounds of the present invention wherein n is 1, R², R⁴, R⁵, R⁷ and R¹¹ are a hydrogen atom, R³ is a chlorine atom, Q is an oxygen atom, and R¹, R⁸, R⁹ and R¹⁰ are the combinations shown in [Table 25] to [Table 27].

In the formula (M6), compounds of the present invention wherein n is 2, R², R⁴, R⁵, R⁷ and R¹¹ are a hydrogen atom, R³ is a chlorine atom, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is a bromine atom, Q is an oxygen atom, and R¹, R⁸, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is a bromine atom, Q is an oxygen atom, and R¹, R⁸, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is a bromine atom, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is a methyl group, Q is an oxygen atom, and R¹, R⁸, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is a methyl group, Q is an oxygen atom, and R¹, R⁶, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is a methyl group, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is a trifluoromethyl group, Q is an oxygen atom, and R¹, R⁸, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is a trifluoromethyl group, Q is an oxygen atom, and R¹, R⁸, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is a trifluoromethyl group, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is a trifluoromethoxy group, Q is an oxygen atom, and R¹, R⁶, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is a trifluoromethoxy group, Q is an oxygen atom, and R¹, R⁸, R⁹ and R²⁰ are the combinations shown in [Table 25] to [Table 27].
In the formula (M6), compounds of the present invention wherein n is 2, R², R⁴, R⁵, R⁷ and R¹¹ are a hydrogen atom, R³ is a trifluoromethoxy group, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is a 2-pyridyl group, Q is an oxygen atom, and R², R⁸, R⁹ and R¹⁰ are the combinations shown in [Table 25 to [Table 27].
In the formula (M6), compounds of the present invention wherein n is 1, R², R⁴, R⁵, R⁷ and R¹¹ are a hydrogen atom, R³ is a 2-pyridyl group, Q is an oxygen atom, and R², R⁸, R⁹ and R¹⁰ are the combinations shown in [Table 25 to [Table 27].
In the formula (M6), compounds of the present invention wherein n is 2, R², R⁴, R⁵, R⁷ and R¹¹ are a hydrogen atom, R³ is a 2-pyridyl group, Q is an oxygen atom, and R¹, R⁸, R⁹ and R¹⁰ are the combinations shown in [Table 25 to [Table 27].
In the formula (M6), compounds of the present invention wherein n is 0, R², R⁴, R⁵, R⁷ and R¹¹ are a hydrogen atom, R³ is a 5-trifluoromethyl-2-pyridyl group, Q is an oxygen atom, and R¹, R⁸, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is a 5-trifluoromethyl-2-pyridyl group, Q is an oxygen atom, and R¹, R⁸, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is a 5-trifluoromethyl-2-pyridyl group, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is a 2-pyrimidinyl group, Q is an oxygen atom, and R¹, R⁸, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is a 2-pyrimidinyl group, Q is an oxygen atom, and R¹, R⁸, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is a 2-pyrimidinyl group, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is a methoxycarbonyl group, Q is an oxygen atom, and R¹, R⁸, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is a methoxycarbonyl group, Q is an oxygen atom, and R¹, R⁸, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is a methoxycarbonyl group, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is an ethoxycarbonyl group, Q is an oxygen atom, and R⁴, R⁸, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is an ethoxycarbonyl group, Q is an oxygen atom, and R⁴, R⁸, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is an ethoxycarbonyl group, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is an aminocarbonyl group, Q is an oxygen atom, and R¹, R⁸, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is an aminocarbonyl group, Q is an oxygen atom, and R¹, R⁸, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is an aminocarbonyl group, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is a methoxycarbonylamino group, Q is an oxygen atom, and R¹, R⁸, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is a methoxycarbonylamino group, Q is an oxygen atom, and R¹, R⁸, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is a methoxycarbonylamino group, Q is an oxygen atom, and R¹, R⁸, R⁹ and R¹⁰ are the combinations shown in [Table 25] to [Table 27].
In the formula (M6), compounds of the present invention wherein n is 0, R², R⁴, R⁵, R⁷ and R¹¹ are a hydrogen atom, R³ is a tert-butoxycarbonylamino group, Q is an oxygen atom, and R¹, R⁸, R⁹ and R¹⁰ are the combinations shown in [Table 25] to [Table 27].
In the formula (M6), compounds of the present invention wherein n is 1, R², R⁴, R⁵, R⁷ and R¹¹ are a hydrogen atom, R³ is a tert-butoxycarbonylamino group, Q is an oxygen atom, and R¹, R⁸, R⁹ and R¹⁰ are the combinations shown in [Table 25] to [Table 27].
In the formula (M6), compounds of the present invention wherein n is 2, R², R⁴, R⁵, R⁷ and R¹¹ are a hydrogen atom, R³ is a tert-butoxycarbonylamino group, Q is an oxygen atom, and R¹, R⁸, R⁹ and R¹⁰ are the combinations shown in [Table 25] to [Table 27].

Examples of the pest on which the composition of the present invention has an effect include arthropod pests such as pest insects and pest mites and nematoda. Specifically, examples of the pests include those shown below.

Hemiptera: Delphacidae such as Laodelphax striatellus, Nilaparvata lugens, and Sogatella furcifera, Deltocephalidae such as Nephotettix cincticeps, Nephotettix virescens, and Empoasca onukii, Aphididae such as Aphis gossypii, Myzus persicae, Brevicoryne brassicae, Aphis spiraecola, Macrosiphum euphorbiae, Aulacorthum solani, Rhopalosiphum padi, Toxoptera citricidus, and Hyalopterus pruni, Pentatomidae such as Nezara antennata, Riptortus clavetus, Leptocorisa chinensis, Eysarcoris parvus, and Halyomorpha mista, Aleyrodidae such as Trialeurodes vaporariorum, Bemisia tabaci, Dialeurodes citri, and Aleurocanthus spiniferus, Coccidae such as Aonidiella aurantii, Comstockaspis perniciosa, Unaspis citri, Ceroplastes rubens, Icerya purchasi, Planococcus kraunhiae, Pseudococcus longispinis, and Pseudaulacaspis pentagona, Tingidae, Cimicoidea such as Cimex lectularius, and Psyliidae.
Lepidoptera: Pyralidae such as Chilo suppressalis, Tryporyza incertulas, Cnaphalocrocis medinalis, Notarcha derogata, Plodia interpunctella, Ostrinia furnacalis, Hellula undalis, and Pediasia teterrellus, Noctuidae such as Spodoptera litura, Spodoptera exigua, Pseudaletia separata, Mamestra brassicae, Agrotis ipsilon, Plusia nigrisigna, Trichoplusia spp., Heliothis spp., and Helicoverpa spp., Pieridae such as Pieris rapae, Adoxophyes spp., Tortricidae such as Grapholitamolesta, Leguminivora glycinivorella, Matsumuraeses azukivora, Adoxophyes orana fasciata, Adoxophyes honmai., Homona magnanima, Archips fuscocupreanus, and Cydia pomonella, Gracillariidae such as Caloptilia theivora and Phyllonorycter ringoneella, Carposinidae such as Carposina niponensis, Lyonetiidae such as Lyonetia spp., Lymantriidae such as Lymantria spp. and Euproctis spp., Yponomeutidae such as Plutella xylostella, Gelechiidae such as Pectinophora gossypiella and Phthorimaea operculella, Arctiidae such as Hyphantria cunea, and Tineidae such as Tinea translucens and Tineola bisselliella.

Thysanoptera: Thripidae such as Frankliniella occidentalis, Thrips parmi, Scirtothrips dorsalis, Thrips tabaci, and Frankliniella intonsa.

Diptera: Culex such as Culex pipiens pallens, Culex tritaeniorhynchus, and Culex guinguefasciatus, Aedes spp. such as Aedes aegypti and Aedes albopictus, Anopheles spp. such as Anopheles sinensis, Chironomidae, Muscidae such as Musca domestica and Muscina stabulans, Calliphoridae, Sarcophagidae, Fanniidae, Anthomyiidae such as Delia platura and Delia antiqua, Agromyzidae such as Agromyza oryzae, Hydrellia griseola, Liriomyza sativae, Liriomyza trifolii, and Chromatomyia horticola, Chloropidae such as Chlorops oryzae, Tephritidae such as Dacus cucurbitae and Ceratitis capitata, Drosophilidae, Phoridae such as Megaselia spiracularis, Psychodidae such as Clogmia albipunctata, Sciaridae, Simuliidae, Tabanidae such as Tabanus trigonus, Stomoxys, and Stomoxyidae.

Coleoptera: Corn rootworm such as Diabrotica virgifera virgifera and Diabrotica undecimpunctata howardi, Scarabaeidae such as Anomala cuprea, Anomala rufocuprea, and Popillia japonica, Curculionidae such as Sitophilus zeamais, Lissorhoptrus oryzophilus, Callosobruchuys chienensis, Echinocnemus squameus, Anthonomus grandis, and Sphenophorus venatus, Tenebrionidae such as Tenebrio molitor and Tribolium castaneum, Chrysomelidae such as Oulema oryzae, Aulacophora femoralis, Phyllotreta striolata, and Leptinotarsa decemlineata, Dermestidae such as Anthrenus verbasci and Dermestes maculates, Anobiidae such as Lasioderma serricorne, Epilachna such as Epilachna vigintioctopunctata, Lyctidae such as Lyctus brunneus and Tomicus piniperda, Bostrychidae, Ptinidae, Cerambycidae such as Anoplophora malasiaca, Agriotes spp., and Paederus fuscipes.

Orthoptera: Locusta migratoria, Gryllotalpa africana, Oxya yezoensis, Oxya japonica, and Grylloidea.

Siphonaptera: Ctenocephalides felis, Ctenocephalides canis, Pulex irritans, Xenopsylla cheopis, and the like.

Anoplura: Pediculus humanus corporis, Pediculus humanus humanus, Phthirus pubis, Haematopinus eurysternus, Dalmalinia ovis, Haematopinus suis, Linognathus setosus, and the like. Mallophaga: Dalmalinia ovis, Dalmalinia bovis, Menopon gallinae, Trichodectes canis, Felicola subrostrata, and the like.

Hymenoptera: Formicidae such as Monomorium pharaosis, Formica furca japonica, Ochetellus glaber, Pristomyrmex pungens, Pheidole noda, Acromyrmex spp., Solenopsis spp., and Linepithema humile, Vespidae, Bethylidae, and Tenthredinidae such as Athalia rosae and Athalia japonica.

Nematoda: Aphelenchoides besseyi, Nothotylenchus acris, Meloidogyne incognita, Meloidogyne hapla, Meloidogyne javanica, Heterodera glycines, Globodera rostochiensis, Pratylenchus coffeae, and Pratylenchus neglectus.

Blattodea: Blattella germanica, Periplaneta fuliginosa, Periplaneta americana, Periplaneta brunnea, and Blatta orientalis.

Isoptera: Reticulitermes speratus, Coptotermes formosanus, Incisitermes minor, Cryptotermes domesticus, Odontotermes formosanus, Neotermes koshunensis, Glyptotermes satsumensis, Glyptotermes nakajimai, Glyptotermes fuscus, Glyptotermes kodamai, Glyptotermes kushimensis, Hodotermopsis japonica, Coptotermes guangzhoensis, Reticulitermes miyatakei, Reticulitermes flaviceps amamianus, Reticulitermes sp., Nasutitermes takasagoensis, Pericapritermes nitobei, Sinocapritermes mushae, and the like.

Acarina: Tetranychidae such as Tetranychus urticae, Tetranychus kanzawai, Panonychus citri, Panonychus ulmi, and Oligonychus spp., Eriophyidae such as Aculops pelekassi, Phyllocoptruta citri, Aculops lycopersici, Calacarus carinatus, Acaphylla theavagrans, Eriophyes chibaensis, and Aculus schlechtendali, Tarsonemidae such as Polyphagotarsonemus latus, Tenuipalpidae such as Brevipalpus phoenicis, Tuckerellidae, Metastigmata such as Haemaphysalis longicornis, Haemaphysalis flava, Dermacentor taiwanicus, Dermacentor variabilis, Ixodes ovatus, Ixodes persulcatus, Ixodes scapularis, Amblyomma americanum, Boophilus microplus, and Rhipicephalus sanguineus, Acaridae such as Tyrophagus putrescentiae and Tyrophagus similis, Pyroglyphidae such as Dermatophagoides farinae and Dermatophagoides ptrenyssnus, Cheyletidae such as Cheyletus eruditus, Cheyletus malaccensis, Cheyletus moorei, and Cheyletiella yasguri, Sarcoptidae such as Octodectes cynotis and Sacroptes scabiei, Demodicidae such as Demodex canis, Listrophoridae, Oribatei, Dermanyssidae such as Ornithonyssus bacoti, Ornithonyssus sylvairum, and Dermanyssus gallinae, Trombiculidae such as Leptotrombidium akamushi, Arachnida such as Chiracanthium japonicum and Latrodectus hasseltii, and the like.

Chilopoda: Thereuonema hilgendorfi, Scolopendra subspinipes, and the like.

Diplopoda: Oxidus gracilis, Nedyopus tambanus, and the like.

Isopoda: Armadillidium vulgare, and the like.

Gastropoda: Limax marginatus, Limaxflavus, and the like.

The pest control agent of the present invention contains the compound of the present invention and an inert carrier. The pest control agent of the present invention is usually obtained by mixing the compound of the present invention and an inert carrier such as a solid carrier, a liquid carrier or a gaseous carrier, and adding a surfactant or other auxiliaries for formulation as necessary, to be formulated into emulsifiable concentrates, oil formulations, dust formulations, granules, wettable powders, flowables, microcapsule formulations, aerosols, smoking agents, poisonous baits, resin formulations, shampoo agents, paste formulations, foam agents, carbon dioxide preparations, tablets, and the like. These formulations may be processed into mosquito repellent coils, electric mosquito repellent mats, mosquito repellent liquid formulations, smoking agents, fumigants, sheet formulations, spot-on agents, or oral treatment agents, and used.

The pest control agent of the present invention usually contains the compound of the present invention in an amount of 0.01 to 95% by weight.

Examples of the solid carrier which is used in the formulation include fine powder and granules of clays (kaolin clay, diatomaceous earth, bentonite, Fubasami clay, acid clay, etc.), synthetic hydrated silicon oxide, talc, ceramics, other inorganic minerals (sericite, quartz, sulfur, activated carbon, calcium carbonate, hydrated silica, etc.), fine powder and granulated substances of chemical fertilizers (ammonium sulfate, ammonium phosphate, ammonium nitrate, urea, ammonium chloride, etc.) and the like, and synthetic resins (polyester resins such as polypropylene, polyacrylonitrile, polymethylmethacrylate and polyethylene terephthalate, nylon resins such as nylon-6, nylon-11 and nylon-66, polyamide resin, polyvinyl chloride, polyvinylidene chloride, vinyl chloride-propylene copolymer, and the like).

Examples of the liquid carrier include water, alcohols (methanol, ethanol, isopropyl alcohol, butanol, hexanol, benzyl alcohol, ethylene glycol, propylene glycol, phenoxyethanol, etc.), ketones (acetone, methyl ethyl ketone, cyclohexanone, etc.), aromatic hydrocarbons (toluene, xylene, ethylbenzene, dodecylbenzene, phenylxylylethane, methylnaphthalene, etc.), aliphatic hydrocarbons (hexane, cyclohexane, kerosene, light oil, etc.), esters (ethyl acetate, butyl acetate, isopropyl myristate, ethyl oleate, diisopropyl adipate, diisobutyl adipate, propylene glycol monomethyl ether acetate, etc.), nitriles (acetonitrile, isobutyronitrile, etc.), ethers (diisopropyl ether, 1,4-dioxane, ethylene glycol dimethyl ether, diethylene glycol dimethyl ether, diethylene glycol monomethyl ether, propylene glycol monomethyl ether, dipropylene glycol monomethyl ether, 3-methoxy-3-methyl-1-butanol, etc.), acid amides (DMF, N,N-dimethylacetamide, etc.), halogenated hydrocarbons (dichloromethane, trichloroethane, carbon tetrachloride, etc.), sulfoxides (DMSO, etc.), and propylene carbonate and vegetable oils (soybean oil, cottonseed oil, etc.). Examples of the gaseous carrier include fluorocarbon, butane gas, LPG (liquefied petroleum gas), dimethyl ether, and carbon dioxide.

Examples of the surfactant include nonionic surfactants such as polyoxyethylene alkyl ether, polyoxyethylene alkylaryl ether and polyethylene glycol fatty acid ester, and anionic surfactants such as alkyl sulfonates, alkylbenzene sulfonates and alkylsulfates.

The other auxiliaries for formulation include such as fixing agents, dispersants, colorants and stabilizers, specifically, for example, casein, gelatin, polysaccharides (starch, arabic gum, cellulose derivatives, alginic acid, etc.), lignin derivatives, bentonite, synthetic water-soluble polymers (polyvinyl alcohol, polyvinyl pyrrolidone, polyacrylic acid, etc.), PAP (isopropyl acid phosphate), BHT (2,6-di-tert-butyl-4-methylphenol) and PHA (mixtures of 2-tert-butyl-4-methoxyphenol and 3-tert-butyl-4-methoxyphenol).

Examples of a base material of the resin formulation include vinyl chloride polymer, polyurethane and the like, and a plasticizer such as ester phthalates (dimethyl phthalate, dioctyl phthalate, etc.), ester adipates or stearic acid may be added to these base materials as necessary. The resin formulation is obtained by kneading a compound into the base material using an ordinary kneading apparatus, and then molding it by injection molding, extrusion molding, press molding or the like, and can be processed into a plate, film, taped, reticular or string resin formulation by further undergoing molding or cutting step as necessary. These resin formulation is processed into, for example, a collar for animal, an ear tag for animal, a sheet formulation, an induction cord, and a gardening pole.

Examples of a base material of the poisonous bait include grain powder, vegetable oil, sugar, crystalline cellulose and the like, and further, an antioxidant such as dibutylhydroxytoluene and nordihydroguaiaretic acid, a preservative such as dehydroacetic acid, a substance for preventing accidental ingestion by children and pets such as red pepper powder, a pest attractant such as cheese flavor, onion flavor and peanut oil or the like are added as necessary.

The method for controlling pests of the present invention is carried out by applying an effective amount of the compound of the present invention to a pest directly and/or a pest-infested area (plants, soil, in-house, animal body, etc.). In the method for controlling pests of the present invention, the compound is usually used in the form of the pest control agent of the present invention.

When the pest control agent of the present invention is used in pest controlling in the agricultural field, the application amount is usually 1 to 10000 g per the amount of the compound of the present invention per 10000 m². When the pest control agent of the present invention is formulated into an emulsifiable concentrate, a wettable powder, a flowable or the like, the pest control agent is usually diluted with water for an application so as to have a concentration of the active ingredient of 0.01 to 10000 ppm, and dust formulations, granules and the like are usually applied as they are.

These formulations and formulation solutions diluted with water may be directly applied by being sprayed on a pest or a plant such as crops which should be protected from pests, and also may be applied on a soil in order to control a pest that infests in the soil of cultivated land.

Also, the resin formulation processed into a sheet or string can be also applied by a method such as winding it around crops, spreading it in the vicinity of crops, or spreading it to the soil around crop roots.

When the pest control agent of the present invention is used in controlling the pest that inhabits in the house, the application amount is usually 0.01 to 1000 mg in an amount of the compound of the present invention per 1 m² of an area to be treated, in the case of using it on a planar area, and is usually 0.01 to 500 mg in an amount of the compound of the present invention per 1 m³ of a space to be treated, in the case of using it in a space. When the pest control agent of the present invention is formulated into an emulsifiable concentrate, a wettable powder, a flowable or the like, the pest control agent is usually diluted with water for an application so as to have a concentration of the active ingredient of 0.1 to 10000 ppm, and oil formulations, aerosols, smoking agents, poisonous baits and the like are applied as they are.

When the arthropod pest control agent of the present invention is used in the control of external parasites on livestock such as cows, horses, pigs, sheep, goats and chickens, and small animals such as dogs, cats, rats and mice, veterinary known methods can be applied to the animals. As specific methods, the formulation is administered, for example, byway of a tablet, mixing in feed, a suppository and injection (intramuscular, subcutaneous, intravenous, intraperitoneal injections, etc.), when systemic control is intended, and the formulation is used, for example, by way of spraying an oil solution or aqueous solution, pour-on or spot-on treatment, washing an animal with a shampoo formulation, or putting a collar or ear tag made of a resin formulation on to an animal, when non-systemic control is intended. The amount of the compound of the present invention when administered to an animal body is usually in the range from 0.1 to 1000 mg per 1 kg of the weight of an animal.

The pest control agent of the present invention can be used in the farmland where the following “crops” are grown.

Crops: corn, rice, wheat, barley, rye, oat, sorghum, cotton, soybean, peanut, sarrazin, sugar beet, rapeseed, sunflower, sugar cane, tobacco, etc.

Vegetables: Solanaceae vegetables (eggplant, tomato, green pepper, hot pepper, potato, etc.), Cucurbitaceae vegetables (cucumber, pumpkin, zucchini, watermelon, melon, etc.), Cruciferae vegetables (Japanese radish, turnip, horseradish, kohlrabi, Chinese cabbage, cabbage, brown mustard, broccoli, cauliflower, etc.), Compositae vegetables (burdock, garland chrysanthemum, artichoke, lettuce, etc.), Liliaceae vegetables (Welsh onion, onion, garlic, asparagus, etc.), Umbelliferae vegetables (carrot, parsley, celery, parsnip, etc.), Chenopodiaceae vegetables (spinach, Swiss chard, etc.), Labiatae vegetables (Japanese mint, mint, basil, etc.), strawberry, sweat potato, yam, aroid, etc.

Fruit trees: pomaceous fruits (apple, common pear, Japanese pear, Chinese quince, quince, etc.), stone fleshy fruits (peach, plum, nectarine, Japanese plum, cherry, apricot, prune, etc.), citrus plants (Satsuma mandarin, orange, lemon, lime, grapefruits, etc.), nuts (chestnut, walnut, hazel nut, almond, pistachio, cashew nut, macadamia nut, etc.), berry fruits (blueberry, cranberry, blackberry, raspberry, etc.), grape, persimmon, olive, loquat, banana, coffee, date, coconut, oil palm, etc.

Trees other than fruit trees: tea, mulberry, flowering trees and shrubs (azalea, camellia, hydrangea, sasanqua, Illicium religiosum, cherry tree, tulip tree, crape myrtle, fragrant olive, etc.), street trees (ash tree, birch, dogwood, eucalyptus, ginkgo, lilac, maple tree, oak, poplar, cercis, Chinese sweet gum, plane tree, zelkova, Japanese arborvitae, fir tree, Japanese hemlock, needle juniper, pine, spruce, yew, elm, horse-chestnut, etc.), sweet viburnum, Podocarpus macrophyllus, Japanese cedar, Japanese cypress, croton, spindle tree, Chainese howthorn, etc.

Lawn: zoysia (Japanese lawn grass, mascarene grass, etc.), Bermuda grass (Cynodon dactylon, etc.), bent grass (creeping bent grass, Agrostis stolonifera, Agrostis tennis, etc.), bluegrass (Kentucky bluegrass, rough bluegrass, etc.), fescue (tall fescue, chewing fescue, creeping fescue, etc.), ryegrass (darnel, perennial ryegrass, etc.), cocksfoot, timothy grass, etc.

Others: flowers (rose, carnation, chrysanthemum, Eustoma grandiflorum. Shinners (prairie gentian), gypsophila, gerbera, pot marigold, salvia, petunia, verbena, tulip, aster, gentian, lily, pansy, cyclamen, orchid, lily of the valley, lavender, stock, ornamental kale, primula, poinsttia, gladiolus, cattleya, daisy, cymbidium, begonia, etc.), bio-fuel plants (Jatropha, curcas, safflower, Camelina alyssum, switchgrass, miscanthus, reed canary grass, Arundo donax, kenaf, cassava, willow, algae, etc.), foliage plants, etc.

The “crops” also contains genetically modified crops.

The pest control agent of the present invention can be used as a mixture with or in combination with other insecticide, miticide, nematicide, fungicide, plant growth regulator, herbicide or synergist. Examples of the active ingredient of said insecticide, miticide, nematicide, fungicide, herbicide and synergist are shown below.

### Active Ingredients of Insecticide

### (1) Organic Phosphorus Compounds

acephate, Aluminium phosphide, butathiofos, cadusafos, chlorethoxyfos, chlorfenvinphos, chlorpyrifos, chlorpyrifos-methyl, cyanophos: CYAP, diazinon, DCIP (dichlorodiisopropyl ether), dichlofenthion: ECP, dichlorvos: DDVP, dimethoate, dimethylvinphos, disulfoton, EPN, ethion, ethoprophos, etrimfos, fenthion: MPP, fenitrothion: MEP, fosthiazate, formothion, Hydrogen phosphide, isofenphos, isoxathion, malathion, mesulfenfos, methidathion: DMTP, monocrotophos, naled: PRP, oxydeprofos: ESP, parathion, phosalone, phosmet: PMP, pirimiphos-methyl, pyridafenthion, quinalphos, phenthoate: PAP, profenofos, propaphos, prothiofos, pyraclorfos, salithion, sulprofos, tebupirimfos, temephos, tetrachloryinphos, terbufos, thiometon, trichlorphon: DEP, vamidothion, phorate, and cadusafos.

### (2) Carbamate Compounds

alanycarb, bendiocarb, benfuracarb, BPMC, carbaryl, carbofuran, carbosulfan, cloethocarb, ethiofencarb, fenobucarb, fenothiocarb, fenoxycarb, furathiocarb, isoprocarb: MIPC, metolcarb, methomyl, methiocarb, NAC, oxamyl, pirimicarb, propoxur: PHC, XMC, thiodicarb, xylylcarb, and aldicarb.

### (3) Pyrethroid Compounds

acrinathrin, allethrin, benfluthrin, beta-cyfluthrin, bifenthrin, cycloprothrin, cyfluthrin, cyhalothrin, cypermethrin, deltamethrin, esfenvalerate, ethofenprox, fenpropathrin, fenvalerate, flucythrinate, flufenoprox, flumethrin, fluvalinate, halfenprox, imiprothrin, permethrin, prallethrin, pyrethrins, resmethrin, sigma-cypermethrin, silafluofen, tefluthrin, tralomethrin, transfluthrin, tetramethrin, phenothrin, cyphenothrin, alpha-cypermethrin, zeta-cypermethrin, lambda-cyhalothrin, gamma-cyhalothrin, furamethrin, tau-fluvalinate, metofluthrin, profluthrin, dimefluthrin, 2,3,5,6-tetrafluoro-4-(methoxymethyl)benzyl (EZ)-(1RS,3RS;1RS,3SR)-2,2-dimethyl-3-prop-1-enylcyclopropanecarboxylate, 2,3,5,6-tetrafluoro-4-methylbenzyl (EZ)-(1RS, 3RS; 1RS, 3SR)-2,2-dimethyl-3-prop-1-enylcyclopropanecarboxylate, and 2,3,5,6-tetrafluoro-4-(methoxymethyl)benzyl (1RS, 3RS; 1RS, 3SR)-2,2-dimethyl-3-(2-methyl-1-propenyl)cyclopropanecarboxylate, 2,3,5,6-tetrafluoro-4-(methoxymethyl)benzyl(EZ)-(1RS, 3RS; 1RS, 3SR)-2,2-dimethyl-3-(2-cyano-1-propenyl)cyclopropanecarboxylate.

### (4) Nereistoxin Compounds

cartap, bensultap, thiocyclam, monosultap, and bisultap.

### (5) Neonicotinoid Compounds

imidacloprid, nitenpyram, acetamiprid, thiamethoxam, thiacloprid, dinotefuran, and clothianidin.

### (6) Benzoyl Urea Compounds

chlorfluazuron, bistrifluron, diafenthiuron, diflubenzuron, fluazuron, flucycloxuron, flufenoxuron, hexaflumuron, lufenuron, novaluron, noviflumuron, teflubenzuron, ttiflumuron, and triazuron.

### (7) Phenylpyrazole-Based Compounds

acetoprole, ethiprole, fipronil, vaniliprole, pyriprole, and pyrafluprole.

### (8) Bt Toxins

Living spores derived from Bacillus thuringiensis and produced crystalline toxins and mixtures thereof.

### (9) Hydrazine Compounds

chromafenozide, halofenozide, methoxyfenozide, and tebufenozide.

### (10) Organic Chlorine Compounds

aldrin, dieldrin, dienochlor, endosulfan, and methoxychlor.

### (11) Other Active Ingredients of Insecticide

machine oil, nicotine-sulfate; avermectin-B, bromopropylate, buprofezin, chlorphenapyr, cyantraniliprole, cyromazine, D-D(1,3-Dichloropropene), emamectin-benzoate, fenazaquin, flupyrazofos, hydroprene, methoprene, indoxacarb, metoxadiazone, milbemycin-A, pymetrozine, pyridalyl, pyriproxyfen, spinosad, sulfluramid, tolfenpyrad, triazamate, flubendiamide, lepimectin, Arsenic acid, benclothiaz, Calcium cyanamide, Calcium polysulfide, chlordane, DDT, DSP, flufenerim, flonicamid, flurimfen, formetanate, metam-ammonium, metam-sodium, Methyl bromide, Potassium oleate, protrifenbute, spiromesifen, sulfoxaflor, Sulfur, metaflumizone, spirotetramat, pyrifluquinazone, spinetoram, chlorantraniliprole, tralopyril, cyantraniliprole, compounds represented by the following formula (K):

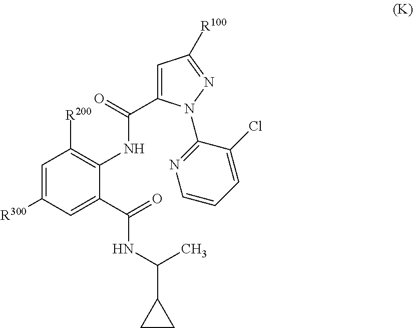

wherein R¹⁰⁰ represents chlorine, bromine or a trifluoromethyl group,

R²⁰⁰ represents chlorine, bromine or a methyl group, and

R³⁰⁰ represents chlorine, bromine or a cyano group, and compounds represented by the following formula (L):

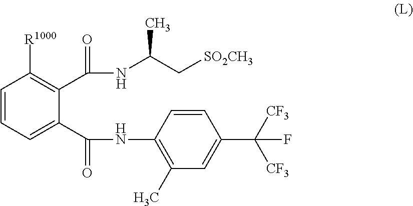

wherein R¹⁰⁰⁰ represents chlorine, bromine or iodine.
Active ingredients of Miticide

acequinocyl, amitraz, benzoximate, bifenaate, bromopropylate, chinomethionat, chlorobenzilate, CPCBS (chlorfenson), clofentezine, cyflumetofen, kelthane (dicofol), etoxazole, fenbutatin oxide, fenothiocarb, fenpyroximate, fluacrypyrim, fluproxyfen, hexythiazox, propargite: BPPS, polynactins, pyridaben, Pyrimidifen, tebufenpyrad, tetradifon, spirodiclofen, spiromesifen, spirotetramat, amidoflumet, and cyenopyrafen.

Active ingredients of Nematicide

DCIP, fosthiazate, levamisol, methyisothiocyanate, morantel tartarate, and imicyafos.

Active ingredients of Fungicide

azole fungicidal compounds such as propiconazole, prothioconazole, triadimenol, prochloraz, penconazole, tebuconazole, flusilazole, diniconazole, bromuconazole, epoxiconazole, difenoconazole, cyproconazole, metconazole, triflumizole, tetraconazole, myclobutanil, fenbuconazole, hexaconazole, fluquinconazole, triticonazole, bitertanol, imazalil, and flutriafol;

Cyclic amine fungicidal compounds such as fenpropimorph, tridemorph, and fenpropidin;
Benzimidazole fungicidal compounds such as carbendezim, benomyl, thiabendazole, and thiophanate-methyl; procymidone; cyprodinil; pyrimethanil; diethofencarb; thiuram; fluazinam; mancozeb; iprodione; vinclozolin; chlorothalonil; captan; mepanipyrim; fenpiclonil; fludioxonil; dichlofluanid; folpet; kresoxim-methyl; azoxystrobin; trifloxystrobin; fluoxastrobin; picoxystrobin; pyraclostrobin; dimoxystrobin; pyribencarb; spiroxamine; quinoxyfen; fenhexamid; famoxadone; fenamidone; zoxamide; ethaboxam; amisulbrom; iprovalicarb; benthiavalicarb; cyazofamid; mandipropamid; boscalid; penthiopyrad; metrafenone; fluopiran; bixafen; cyflufenamid; proquinazid; isotianil and tiadinil;
Active ingredients of Herbicide

### (1) Phenoxy Fatty Acid Herbicidal Compounds

2,4-PA, MCP, MCPB, phenothiol, mecoprop, fluroxypyr, triclopyr, clomeprop, and naproanilide.

### (2) Benzoate Herbicidal Compounds

2,3,6-TBA, dicamba, clopyralid, picloram, aminopyralid, quinclorac, and quinmerac.

### (3) Urea Herbicidal Compounds

diuron, linuron, chlortoluron, isoproturon, fluometuron, isouron, tebuthiuron, methabenzthiazuron, cumyluron, daimuron, and methyl-daimuron.

### (4) Triazine Herbicidal Compounds

atrazine, ametoryn, cyanazine, simazine, propazine, simetryn, dimethametryn, prometryn, metribuzin, triaziflam, and indaziflam.

### (5) Pipyridinium Herbicidal Compounds

paraquat, and diquat.

### (6) Hydroxybenzonitrile Herbicidal Compounds

bromoxynil, and ioxynil.

### (7) Dinitroaniline Herbicidal Compounds

pendimethalin, prodiamine, and trifluralin.

### (8) Organophosphorus Herbicidal Compounds

amiprofos-methyl, butamifos, bensulide, piperophos, anilofos, glyphosate, glufosinate, glufosinate-P, and bialaphos.

### (9) Carbamate Herbicidal Compounds

di-allate, tri-allate, EPIC, butylate, benthiocarb, esprocarb, molinate, dimepiperate, swep, chlorpropham, phenmedipham, phenisopham, pyributicarb, and asulam.

### (10) Acid Amide Herbicidal Compounds

propanil, propyzamide, bromobutide, and etobenzanid.

### (11) Chloroacetanilide Herbicidal Compounds

acetochlor, alachlor, butachlor, dimethenamid, propachlor, metazachlor, metolachlor, pretilachlor, thenylchlor, and pethoxamid.

### (12) Diphenyl Ether Herbicidal Compounds

acifluorfen-sodium, bifenox, oxyfluorfen, lactofen, fomesafen, chlomethoxynil, and aclonifen.

### (13) Cyclic Imide Herbicidal Compounds

oxadiazon, cinidon-ethyl, carfentrazone-ethyl, surfentrazone, flumiclorac-pentyl, flumioxazin, pyraflufen-ethyl, oxadiargyl, pentoxazone, fluthiacet-methyl, butafenacil, benzfendizone, bencarbazone, and saflufenacil.

### (14) Pyrazole Herbicidal Compounds

benzofenap, pyrazolate, pyrazoxyfen, topramezone, and pyrasulfotole.

### (15) Triketone Herbicidal Compounds

isoxaflutole, benzobicyclon, sulcotrione, mesotrione, tembotrione, and tefuryltrione.

### (16) Aryloxyphenoxypropionate Herbicidal Compounds

clodinafop-propargyl, cyhalofop-butyl, diclofop-methyl, fenoxaprop-ethyl, fluazifop-butyl, haloxyfop-methyl, and quizalofop-ethyl, metamifop.

### (17) Triune Oxime Herbicidal Compounds

alloxydim-sodium, sethoxydim, butroxydim, clethodim, cloproxydim, cycloxydim, tepraloxydim, tralkoxydim, and profoxydim.

### (18) Sulfonyl Urea Herbicidal Compounds

chlorsulfuron, sulfometuron-methyl, metsulfuron-methyl, chlorimuron-ethyl, tribenuron-methyl, triasulfuron, metsulfuron-methyl, thifensulfuron-methyl, pyrazosulfuron-ethyl, primisulfuron-methyl, nicosulfuron, amidosulfuron, cinosulfuron, imazosulfuron, rimsulfuron, halosulfuron-methyl, prosulfuron, ethametsulfuron-methyl, triflusulfuron-methyl, flazasulfuron, cyclosulfamuron, flupyrsulfuron, sulfosulfuron, azimsulfuron, ethoxysulfuron, oxasulfuron, iodosulfuron-methyl-sodium, foramsulfuron, mesosulfuron-methyl, trifloxysulfuron, tritosulfuron, orthosulfamuron, flucetosulfuron, and propyrisulfuron.

### (19) Imidazolinone Herbicidal Compounds

imazamethabenz-methyl, imazamethapyr, imazamox, imazapyr, imazaquin, and imazethapyr.

### (20) Sulfonamide Herbicidal Compounds

flumetsulam, metosulam, diclosulam, florasulam, cloransulam-methyl, penoxsulam, and pyroxsulam.

### (21) Pyrimidinyloxybenzoate Herbicidal Compounds

pyrithiobac-sodium, bispyribac-sodium, pyriminobac-methyl, pyribenzoxim, pyriftalid, and pyrimisulfan.

### (22) Other Herbicidal Compounds

bentazon, bromacil, terbacil, chlorthiamid, isoxaben, dinoseb, amitrole, cinmethylin, tridiphane, dalapon, diflufenzopyr-sodium, dithiopyr, thiazopyr, flucarbazone-sodium, propoxycarbazone-sodium, mefenacet, flufenacet, fentrazamide, cafenstrole, indanofan, oxaziclomefone, benfuresate, ACN, pyridate, chloridazon, norflurazon, flurtamone, diflufenican, picolinafen, beflubutamid, clomazone, amicarbazone, pinoxaden, pyraclonil, pyroxasulfone, thiencarbazone-methyl, aminocyclopyrachlor, ipfencarbazone, and methiozolin.
Active ingredients of Synergist

piperonyl butoxide, sesamex, sulfoxide, N-(2-ethylhexyl)-8,9,10-trinorborn-5-ene-2,3-dicarboximide (MGK 264), N-declyimidazole), WARF-antiresistant, TEPT, TPP, IEP, PSCP, CH₃I, t-phenylbutenone, diethylmaleate, DMC, FDMC, ETP, and ETN.

### EXAMPLES

Hereinbelow, the present invention will be further described in detail by production examples, reference examples, formulation examples, test examples, and the like. However, the present invention is not limited to these examples.

First, the production examples for the production of the compounds of the present invention are shown below.

### Production Example 1 (1)

1.5 mL of ethyl mercaptan was added to a mixture of 3.83 g of 2-fluoro-4-trifluoromethylbenzoic acid, 10 mL of NMP and 15 g of cesium carbonate, and the mixture was stirred at 60° C. for 4 hours. A saturated aqueous sodium bicarbonate solution was poured to the cooled reaction mixture, and the mixture was extracted with ethyl acetate. Hydrochloric acid was added to the aqueous layer, and the precipitated solid was filtered and dried under reduced pressure to obtain 4.6 g of 2-ethylsulfanyl-4-trifluoromethylbenzoic acid.

### 2-Ethylsulfanyl-4-trifluoromethylbenzoic acid

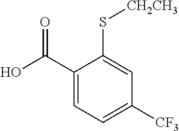 [SMILES: CCSc1cc(C(F)(F)F)ccc1C(=O)O]

¹H-NMR (CDCl₃) δ: 8.22 (1H, d), 7.55 (1H, s), 7.43 (1H, d), 3.02 (2H, q), 1.43 (3H, t).

### Production Example 1 (2)

A mixture of 0.32 g of 4-trifluoromethylaniline, 0.50 g of 2-ethylsulfanyl-4-trifluoromethylbenzoic acid, 0.57 g of EDCI hydrochloride, 0.02 g of HOBt and 5 ml of pyridine was stirred at 60° C. for 2 hours. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting solid was washed with hexane to obtain 0.72 g of 2-ethylsulfanyl-4-trifluoromethyl-N-(4-trifluoromethyl-phen yl)-benzamide.

### 2-Ethylsulfanyl-4-trifluoromethyl-N-(4-trifluoromethyl-phen yl)-benzamide

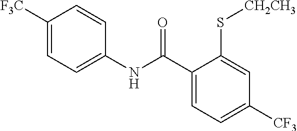 [SMILES: CCSc1cc(C(F)(F)F)ccc1C(=O)Nc1ccc(C(F)(F)F)cc1]

¹H-NMR (CDCl₃) δ: 8.75 (1H, s), 7.92 (1H, d), 7.79 (2H, d), 7.70 (1H, s), 7.65 (2H, d), 7.58 (1H, d), 3.01 (2H, q), 1.33 (3H, t).

### Production Example 1 (3)

0.23 g of potassium carbonate and 0.35 mL of methyl iodide were added to a mixture of 0.45 g of 2-ethylsulfanyl-4-trifluoromethyl-N-(4-trifluoromethyl-phen yl)-benzamide and 6 mL of acetone, and the mixture was stirred at 60° C. for 5 hours. 0.35 mL of methyl iodide and 2 mL of DMF were added to the cooled reaction mixture, and the mixture was stirred at 60° C. for 5 hours. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting residue was applied to a silica gel column chromatography to obtain 0.38 g of 2-ethylsulfanyl-N-methyl-4-trifluoromethyl-N-(4-trifluoromethyl-phenyl)-benzamide (hereinafter, referred to as Compound of Present Invention 1).

### Compound of Present Invention 1

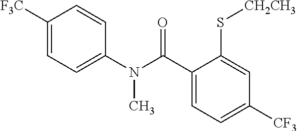 [SMILES: CCSc1cc(C(F)(F)F)ccc1C(=O)N(C)c1ccc(C(F)(F)F)cc1]

¹H-NMR (CDCl₃) δ: 7.75-6.92 (7H, m), 3.50 (3H, brs), 3.09-2.90 (2H, m), 1.40-1.30 (3H, m).

### Production Example 2 (1)

A mixture of 0.35 g of 4-trifluoromethoxyaniline, 0.50 g of 2-ethylsulfanyl-4-trifluoromethylbenzoic acid, 0.57 g of EDCI hydrochloride, 0.02 g of HOBt and 5 ml of pyridine was stirred at 60° C. for 2 hours. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting solid was washed with hexane to obtain 0.75 g of 2-ethylsulfanyl-N-(4-trifluoromethoxyl-phenyl)-4-trifluoromethyl-benzamide.

### 2-Ethylsulfanyl-N-(4-trifluoromethoxyl-phenyl)-4-trifluoromethyl-benzamide

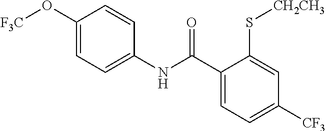 [SMILES: CCSc1cc(C(F)(F)F)ccc1C(=O)Nc1ccc(OC(F)(F)F)cc1]

¹H-NMR (CDCl₃) δ: 8.60 (1H, s), 7.90 (1H, d), 7.74-7.64 (3H, m), 7.57 (1H, d), 7.25 (2H, d), 3.01 (2H, q), 1.33 (3H, t).

### Production Example 2 (2)

0.22 g of potassium carbonate and 0.34 mL of methyl iodide were added to a mixture of 0.45 g of 2-ethylsulfanyl-N-(4-trifluoromethoxyl-phenyl)-4-trifluoromethyl-benzamide and 6 mL of acetone, and the mixture was stirred at 60° C. for 5 hours. 0.34 mL of methyl iodide and 2 mL of DMF were added to the cooled reaction mixture, and the mixture was stirred at 60° C. for 5 hours. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting residue was applied to a silica gel column chromatography to obtain 0.42 g of 2-ethylsulfanyl-N-methyl-N-(4-trifluoromethoxyl-phenyl)-4-t rifluoromethyl-benzamide (hereinafter, referred to as Compound of Present Invention 2).

### Compound of Present Invention 2

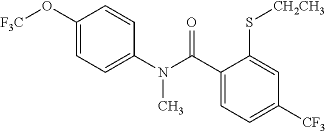 [SMILES: CCSc1cc(C(F)(F)F)ccc1C(=O)N(C)c1ccc(OC(F)(F)F)cc1]

¹H-NMR (CDCl₃) δ: 7.70-6.92 (7H, m), 3.47 (3H, brs), 2.99 (2H, q), 1.33 (3H, t).

### Production Example 3 (1)

A mixture of 0.42 g of 4-pentafluoroethylaniline, 0.50 g of 2-ethylsulfanyl-4-trifluoromethylbenzoic acid, 0.57 g of EDCI hydrochloride, 0.02 g of HOBt and 5 ml of pyridine was stirred at 60° C. for 2 hours. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting solid was washed with hexane to obtain 0.75 g of 2-ethylsulfanyl-N-(4-pentafluoroethyl-phenyl)-4-trifluoromethyl-benzamide.

### 2-Ethylsulfanyl-N-(4-pentafluoroethyl-phenyl)-4-trifluoromethyl-benzamide

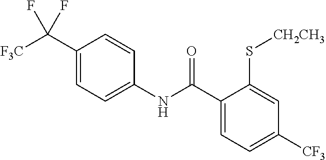 [SMILES: CCSc1cc(C(F)(F)F)ccc1C(=O)Nc1ccc(C(F)(F)C(F)(F)F)cc1]

¹H-NMR (CDCl₃) δ: 8.75 (1H, s), 7.92 (1H, d), 7.81 (2H, d), 7.69 (1H, s), 7.62 (2H, d), 7.59 (1H, d), 3.02 (2H, q), 1.34 (3H, t).

### Production Example 3 (2)

0.25 g of potassium carbonate and 0.38 mL of methyl iodide were added to a mixture of 0.50 g of 2-ethylsulfanyl-N-(4-pentafluoroethyl-phenyl)-4-trifluoromethyl-benzamide and 6 mL of acetone, and the mixture was stirred at 60° C. for 5 hours. 0.38 mL of methyl iodide and 2 mL of DMF were added to the cooled reaction mixture, and the mixture was stirred at 60° C. for 5 hours. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting residue was applied to a silica gel column chromatography to obtain 0.52 g of 2-ethylsulfanyl-N-methyl-N-(4-pentafluoroethyl-phenyl)-4-trifluoromethyl-benzamide (hereinafter, referred to as Compound of Present Invention 3).

### Compound of Present Invention 3

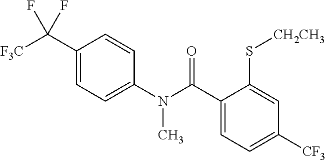 [SMILES: CCSc1cc(C(F)(F)F)ccc1C(=O)N(C)c1ccc(C(F)(F)C(F)(F)F)cc1]

¹H-NMR (CDCl₃) δ: 7.78-7.09 (7H, m), 3.47 (3H, brs), 2.97 (2H, q), 1.32 (3H, t).

### Production Example 4 (1)

A mixture of 0.38 g of 4-trifluoromethylsulfanylaniline, 0.50 g of 2-ethylsulfanyl-4-trifluoromethylbenzoic acid, 0.57 g of EDCI hydrochloride, 0.02 g of HOBt and 5 ml of pyridine was stirred at 60° C. for 2 hours. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting solid was washed with hexane to obtain 0.74 g of 2-ethylsulfanyl-N-(4-trifluoromethylsulfanyl-phenyl)-4-trifluoromethyl-benzamide.

### 2-Ethylsulfanyl-N-(4-trifluoromethylsulfanyl-phenyl)-4-trifluoromethyl-benzamide

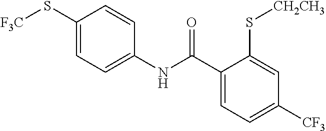 [SMILES: CCSc1cc(C(F)(F)F)ccc1C(=O)Nc1ccc(SC(F)(F)F)cc1]

¹H-NMR (CDCl₃) δ: 8.71 (1H, s), 7.91 (1H, d), 7.75 (2H, d), 7.71-7.65 (3H, m), 7.58 (1H, d), 3.01 (2H, q), 1.34 (3H, t).

### Production Example 4 (2)

0.25 g of potassium carbonate and 0.38 mL of methyl iodide were added to a mixture of 0.50 g of 2-ethylsulfanyl-N-(4-trifluoromethylsulfanyl-phenyl)-4-trifluoromethyl-benzamide and 6 mL of acetone, and the mixture was stirred at 60° C. for 5 hours. 0.38 mL of methyl iodide and 2 mL of DMF were added to the cooled reaction mixture, and the mixture was stirred at 60° C. for 5 hours. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting residue was applied to a silica gel column chromatography to obtain 0.53 g of 2-ethylsulfanyl-N-methyl-4-trifluoromethyl-N-(4-trifluoromethylsulfanyl-phenyl)-benzamide (hereinafter, referred to as Compound of Present Invention 4).

### Compound of Present Invention 4

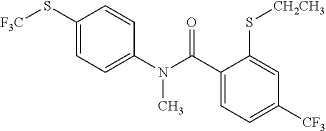 [SMILES: CCSc1cc(C(F)(F)F)ccc1C(=O)N(C)c1ccc(SC(F)(F)F)cc1]

¹H-NMR (CDCl₃) δ: 7.69-7.08 (7H, m), 3.48 (3H, brs), 2.98 (2H, q), 1.32 (3H, t).

### Production Example 5 (1)

A mixture of 0.45 g of 3,5-bis(trifluoromethyl)-aniline, 0.50 g of 2-ethylsulfanyl-4-trifluoromethylbenzoic acid, 0.57 g of EDCI hydrochloride, 0.02 g of HOBt and 5 ml of pyridine was stirred at 60° C. for 2 hours. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting solid was washed with hexane to obtain 0.80 g of N-(3,5-bis-trifluoromethyl-phenyl)-2-ethylsulfanyl-4-trifluoromethyl-benzamide.

### N-(3,5-Bis-trifluoromethyl-phenyl)-2-ethylsulfanyl-4-trifluoromethyl-benzamide

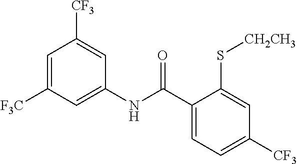 [SMILES: CCSc1cc(C(F)(F)F)ccc1C(=O)Nc1cc(C(F)(F)F)cc(C(F)(F)F)c1]

¹H-NMR (CDCl₃) δ: 9.00 (1H, s), 8.17 (2H, s), 7.95 (1H, d), 7.72 (1H, s), 7.69 (1H, s), 7.61 (1H, d), 3.04 (2H, q), 1.35 (3H, t).

### Production Example 5 (2)

0.25 g of potassium carbonate and 0.38 mL of methyl iodide were added to a mixture of 0.50 g of N-(3,5-bis-trifluoromethyl-phenyl)-2-ethylsulfanyl-4-trifluoromethyl-benzamide and 6 mL of acetone, and the mixture was stirred at 60° C. for 5 hours. 0.38 mL of methyl iodide and 2 mL of DMF were added to the cooled reaction mixture, and the mixture was stirred at 60° C. for 5 hours. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting residue was applied to a silica gel column chromatography to obtain 0.37 g of N-(3,5-bis-trifluoromethyl-phenyl)-2-ethylsulfanyl-N-methyl-4-trifluoromethyl-benzamide (hereinafter, referred to as Compound of Present Invention 5).

### Compound of Present Invention 5

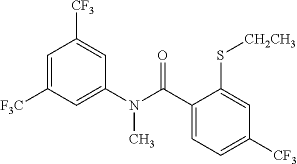 [SMILES: CCSc1cc(C(F)(F)F)ccc1C(=O)N(C)c1cc(C(F)(F)F)cc(C(F)(F)F)c1]

¹H-NMR (CDCl₃) δ: 7.77-7.19 (6H, m), 3.50 (3H, brs), 3.05-2.85 (2H, m), 1.32 (3H, t).

### Production Example 6 (1)

7.9 g of thionyl chloride was added to a mixture of 7.23 g of 4-bromo-2-fluorobenzoic acid, 30 mL of toluene and 0.1 ml of DMF, and the mixture was stirred at 80° C. for 1 hour. The reaction mixture was concentrated under reduced pressure, and 30 ml of toluene, 6.22 g of N-methyl-4-trifluoromethylsulfanylaniline and 7.74 g of diisopropylethylamine were added, and the mixture was stirred at 80° C. for 1 hour. A saturated aqueous sodium bicarbonate solution was poured to the cooled reaction mixture, and the mixture was extracted with ethyl acetate. The organic layer was dried over anhydrous sodium sulfate and then concentrated under reduced pressure, and the resulting residue was applied to a silica gel column chromatography to obtain 12.84 g of N-(4-trifluoromethylsulfanyl-phenyl)-4-bromo-2-fluoro-N-methyl-benzamide.

### N-(4-Trifluoromethylsulfanyl-phenyl)-4-bromo-2-fluoro-N-methyl-benzamide

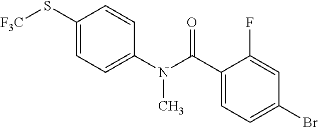 [SMILES: CN(C(=O)c1ccc(Br)cc1F)c1ccc(SC(F)(F)F)cc1]

¹H-NMR (CDCl₃) δ: 7.53 (2H, d), 7.26-7.22 (2H, m), 7.16-7.08 (2H, m), 7.07-7.00 (1H, m), 3.49 (3H, s).

### Production Example 6 (2)

1.05 g of sodium ethanethiolate was added to a mixture of 4.08 g of N-(4-trifluoromethylsulfanyl-phenyl)-4-bromo-2-fluoro-N-methyl-benzamide and 30 ml of DMF under ice cooling, and the mixture was stirred at room temperature for 2 hours. A saturated aqueous sodium chloride solution was poured to the reaction mixture, and the mixture was extracted with ethyl acetate. The organic layer was dried over anhydrous sodium sulfate and then concentrated under reduced pressure, and the resulting residue was applied to a silica gel column chromatography to obtain 3.80 g of a mixture of N-(4-tri fluoromethylsulfanyl-phenyl)-4-bromo-2-fluoro-N-methyl-benzamide, N-(4-tri fluoromethylsulfanyl-phenyl)-4-bromo-2-ethylsulfonyl-N-methyl-benzamide, and N-(4-tri fluoromethylsulfanyl-phenyl)-2-fluoro-4-ethylsulfanyl-N-methyl-benzamide.

3.22 g of m-chloroperbenzoic acid (purity of 68%) was added to a mixture of the crude product obtained above and 20 ml of chloroform under ice cooling, and the mixture was stirred at room temperature for 2 hours. A saturated aqueous sodium bicarbonate solution and a saturated aqueous sodium thiosulfate solution were added to the reaction mixture, and the mixture was extracted with ethyl acetate. The organic layer was dried over anhydrous sodium sulfate and then concentrated under reduced pressure, and the resulting residue was applied to a silica gel column chromatography to obtain 2.00 g of N-(4-trifluoromethylsulfanyl-phenyl)-4-bromo-2-ethylsulfonyl-N-methyl-benzamide (hereinafter, referred to as Compound of Present Invention 88).

### Compound of Present Invention 88

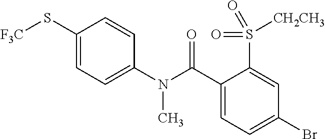 [SMILES: CCS(=O)(=O)c1cc(Br)ccc1C(=O)N(C)c1ccc(SC(F)(F)F)cc1]

¹H-NMR (CDCl₃) δ: 8.24-6.81 (7H, m), 3.66-3.15 (5H, m), 1.39-1.29 (3H, m).

### Production Example 7

Carbon monoxide at 10 atmospheric pressure was introduced to a mixture of 1.77 g of Compound of Present Invention 88, 25 mg of dichlorobis(triphenylphosphine) palladium(II), 60 mg of 1,1′-bis(diphenylphosphino)ferrocene, 0.55 g of potassium carbonate, 1 ml of ethanol and 10 ml of toluene, and the mixture was stirred at 105° C. for 6 hours. The cooled reaction mixture was released into water, and the mixture was extracted with ethyl acetate. The organic layer was dried over anhydrous sodium sulfate and then concentrated under reduced pressure, and the resulting residue was applied to a silica gel column chromatography to obtain 0.27 g of 3-ethylsulfonyl-N-methyl-N-(4-trifluoromethylsulfanyl-phenyl)-ethyl terephthalate (hereinafter, referred to as Compound of Present Invention 87).

### Compound of Present Invention 87

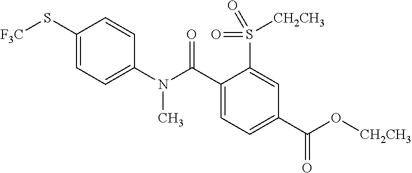 [SMILES: CCOC(=O)c1ccc(C(=O)N(C)c2ccc(SC(F)(F)F)cc2)c(S(=O)(=O)CC)c1]

¹H-NMR (CDCl₃) δ: 8.73-7.00 (7H, m), 4.54-4.27 (2H, m), 3.68-3.15 (5H, m), 1.50-1.23 (3H, m).

### Production Example 8

A mixture of 1.42 g of Compound of Present Invention 87, 0.14 g of lithium hydroxide, 6 ml of THF, 3 ml of methanol and 3 ml of water was stirred at room temperature for 2 hours. A saturated aqueous ammonium chloride solution and a 2 N hydrochloric acid were added to the reaction mixture, and the mixture was extracted with chloroform. The organic layer was dried over anhydrous sodium sulfate and then concentrated under reduced pressure to obtain 1.34 g of 3-ethylsulfonyl-N-methyl-N-(4-trifluoromethylsulfanyl-phenyl)-terephthalic acid (hereinafter, referred to as Compound of Present Invention 91).

### Compound of Present Invention 91

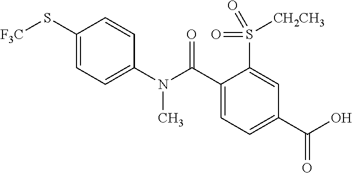 [SMILES: CCS(=O)(=O)c1cc(C(=O)O)ccc1C(=O)N(C)c1ccc(SC(F)(F)F)cc1]

¹H-NMR (CDCl₃) δ: 8.77-8.61 (1H, m), 8.48-8.00 (1H, m), 7.78-7.09 (5H, m), 3.81-3.18 (5H, m), 1.91-1.85 (1H, m), 1.38-1.29 (3H, m).

### Production Method 9

0.66 g of oxalyl chloride was added to a mixture of 1.16 g of Compound of Present Invention 91, 15 ml of chloroform and 0.1 mL of DMF under ice cooling, and the mixture was stirred at room temperature for 2 hours. The reaction mixture was concentrated under reduced pressure, and 9 mL of chloroform was added. 3 ml of the reaction mixture was transferred to another reaction vessel, 0.52 g of aqueous ammonia (28%) was added, and the mixture was stirred at room temperature for 1 hour. A saturated aqueous sodium bicarbonate solution was poured to the reaction mixture, and the mixture was extracted with ethyl acetate. The organic layer was dried over anhydrous sodium sulfate and then concentrated under reduced pressure, and the resulting residue was applied to a silica gel column chromatography to obtain 0.26 g of 3-ethylsulfonyl(4-trifluoromethylsulfanyl-phenyl)-terephthalamide (hereinafter, referred to as Compound of Present Invention 94).

### Compound of Present Invention 94

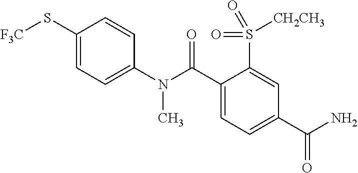 [SMILES: CCS(=O)(=O)c1cc(C(N)=O)ccc1C(=O)N(C)c1ccc(SC(F)(F)F)cc1]

¹H-NMR (CDCl₃) δ: 8.46-8.29 (1H, m), 7.88-7.09 (6H, m), 6.52-5.75 (2H, m), 3.71-3.18 (5H, m), 1.37-1.29 (3H, m).

The compounds described in the production examples described above and the compounds manufactured by the manufacturing method according to the method described in the production examples described above are shown in the tables. The compounds of the present invention represented by the formula (1):

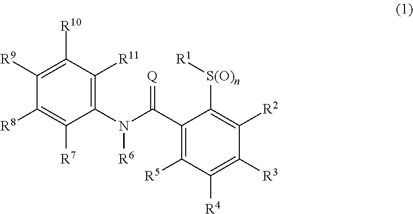

wherein R¹, R², R³, R⁴, R⁵, R⁶, R⁷, R⁸, R⁹, R¹⁰, R¹¹, n and Q represent the combinations shown in [Table 28] to [Table 32] shown below.

TABLE 28

| Compound | | | | | | | | | | | | | |
|---|---|---|---|---|---|---|---|---|---|---|---|---|---|
| of Present | | | | | | | | | | | | | |
| Invention | R¹ | R² | R³ | R⁴ | R⁵ | R⁶ | R⁷ | R⁸ | R⁹ | R¹⁰ | R¹¹ | n | Q |
| 1 | Et | H | CF₃ | H | H | CH₃ | H | H | CF₃ | H | H | 0 | 0 |
| 2 | Et | H | CF₃ | H | H | CH₃ | H | H | OCF₃ | H | H | 0 | 0 |
| 3 | Et | H | CF₃ | H | H | CH₃ | H | H | CF₂CF₃ | H | H | 0 | 0 |
| 4 | Et | H | CF₃ | H | H | CH₃ | H | H | SCF₃ | H | H | 0 | 0 |
| 5 | Et | H | CF₃ | H | H | CH₃ | H | CF₃ | H | CF₃ | H | 0 | 0 |
| 6 | Et | H | CF₃ | H | H | CH₃ | H | Cl | H | Cl | H | 0 | 0 |
| 7 | Et | H | CF₃ | H | H | CH₃ | H | F | H | F | H | 0 | 0 |
| 8 | Et | H | CF₃ | H | H | CH₃ | H | H | CF₃ | H | H | 2 | 0 |
| 9 | Et | H | CF₃ | H | H | CH₃ | H | H | OCF₃ | H | H | 2 | 0 |
| 10 | Et | H | CF₃ | H | H | CH₃ | H | H | CF₂CF₃ | H | H | 2 | 0 |
| 11 | Et | H | CF₃ | H | H | CH₃ | H | H | SCF₃ | H | H | 2 | 0 |
| 12 | Et | H | CF₃ | H | H | CH₃ | H | H | SOCF₃ | H | H | 2 | 0 |
| 13 | Et | H | CF₃ | H | H | CH₃ | H | CF₃ | H | CF₃ | H | 2 | 0 |
| 14 | Et | H | CF₃ | H | H | CH₃ | H | Cl | H | Cl | H | 2 | 0 |
| 15 | Et | H | CF₃ | H | H | CH₃ | H | F | H | F | H | 2 | 0 |
| 16 | Et | H | H | H | H | CH₃ | H | H | CF₃ | H | H | 0 | 0 |
| 17 | Et | H | H | H | H | CH₃ | H | H | OCF₃ | H | H | 0 | 0 |
| 18 | Et | H | H | H | H | CH₃ | H | H | CF₂CF₃ | H | H | 0 | 0 |
| 19 | Et | H | H | H | H | CH₃ | H | H | SCF₃ | H | H | 0 | 0 |
| 20 | Et | H | H | H | H | CH₃ | H | CF₃ | H | CF₃ | H | 0 | 0 |
| 21 | Et | H | H | H | H | CH₃ | H | Cl | H | Cl | H | 0 | 0 |
| 22 | Et | H | H | H | H | CH₃ | H | F | H | F | H | 0 | 0 |

TABLE 29

| Compound | | | | | | | | | | | | | |
|---|---|---|---|---|---|---|---|---|---|---|---|---|---|
| of Present | | | | | | | | | | | | | |
| Invention | R¹ | R² | R³ | R⁴ | R⁵ | R⁶ | R⁷ | R⁸ | R⁹ | R¹⁰ | R¹¹ | n | Q |
| 23 | Et | H | H | H | H | CH₃ | H | H | CF₃ | H | H | 2 | 0 |
| 24 | Et | H | H | H | H | CH₃ | H | H | OCF₃ | H | H | 2 | 0 |
| 25 | Et | H | H | H | H | CH₃ | H | H | CF₂CF₃ | H | H | 2 | 0 |
| 26 | Et | H | H | H | H | CH₃ | H | H | SCF₃ | H | H | 2 | 0 |
| 27 | Et | H | H | H | H | CH₃ | H | CF₃ | H | CF₃ | H | 2 | 0 |
| 28 | Et | H | H | H | H | CH₃ | H | Cl | H | Cl | H | 2 | 0 |
| 29 | Et | H | H | H | H | CH₃ | H | F | H | F | H | 2 | 0 |
| 30 | Et | H | F | H | H | CH₃ | H | H | CF₂CF₃ | H | H | 2 | 0 |
| 31 | Et | H | Cl | H | H | CH₃ | H | H | CF₂CF₃ | H | H | 2 | 0 |
| 32 | Et | H | Br | H | H | CH₃ | H | H | CF₂CF₃ | H | H | 2 | 0 |
| 33 | Et | H | l | H | H | CH₃ | H | H | CF₂CF₃ | H | H | 2 | 0 |
| 34 | Et | H | 2-pyridyl | H | H | CH₃ | H | H | CF₂CF₃ | H | H | 2 | 0 |
| 35 | Et | H | 2-pyrimidyl | H | H | CH₃ | H | H | CF₂CF₃ | H | H | 2 | 0 |
| 36 | Et | H | CH₃ | H | H | CH₃ | H | H | CF₂CF₃ | H | H | 2 | 0 |
| 37 | Et | H | OCH₃ | H | H | CH₃ | H | H | CF₂CF₃ | H | H | 2 | 0 |
| 38 | Et | H | CF₃ | H | H | CH₂CH₃ | H | H | CF₂CF₃ | H | H | 2 | 0 |
| 39 | Et | H | CF₃ | H | H | CycPr | H | H | CF₂CF₃ | H | H | 2 | 0 |
| 40 | Et | H | CF₃ | H | H | CH₂CycPr | H | H | CF₂CF₃ | H | H | 2 | 0 |
| 41 | Et | H | CF₃ | H | H | CH₂CCH | H | H | CF₂CF₃ | H | H | 2 | 0 |
| 42 | Et | H | CF₃ | H | H | COCH₃ | H | H | CF₂CF₃ | H | H | 2 | 0 |
| 43 | Et | H | CF₃ | H | H | CH₃OCH₃ | H | H | CF₂CF₃ | H | H | 2 | 0 |
| 44 | Et | H | CF₃ | H | H | CH₃ | H | H | Br | H | H | 0 | 0 |

TABLE 30

| Compound | | | | | | | | | | | | | |
|---|---|---|---|---|---|---|---|---|---|---|---|---|---|
| of Present | | | | | | | | | | | | | |
| Invention | R¹ | R² | R³ | R⁴ | R⁵ | R⁶ | R⁷ | R⁸ | R⁹ | R¹⁰ | R¹¹ | n | Q |
| 45 | Et | H | CF₃ | H | H | CH₃ | H | H | Br | H | H | 2 | 0 |
| 46 | Et | H | CF₃ | H | H | CH₃ | H | H | CH₃ | H | H | 0 | 0 |
| 47 | Et | H | CF₃ | H | H | CH₃ | H | H | CH₃ | H | H | 2 | 0 |
| 48 | Et | H | CF₃ | H | H | CH₃ | H | H | CH₂CH₃ | H | H | 0 | 0 |
| 49 | Et | H | CF₃ | H | H | CH₃ | H | H | CH₂CH₃ | H | H | 2 | 0 |
| 50 | Et | H | CF₃ | H | H | CH₃ | H | H | CH(CH₃)₂ | H | H | 0 | 0 |
| 51 | Et | H | CF₃ | H | H | CH₃ | H | F | CH(CH₃)₂ | H | H | 2 | 0 |
| 52 | Et | H | CF₃ | H | H | CH₃ | H | H | CF₃ | H | F | 0 | 0 |
| 53 | Et | H | CF₃ | H | H | CH₃ | H | CF₃ | F | H | H | 0 | 0 |
| 54 | Et | H | CF₃ | H | H | CH₃ | H | CF₃ | Cl | H | H | 0 | 0 |
| 55 | Et | H | CF₃ | H | H | CH₃ | H | CF₃ | Br | H | H | 0 | 0 |
| 56 | Et | H | CF₃ | H | H | CH₃ | H | CF₃ | CH₃ | H | H | 0 | 0 |
| 57 | Et | H | CF₃ | H | H | CH₃ | H | CF₃ | OCH₃ | H | H | 0 | 0 |
| 58 | Et | H | CF₃ | H | H | CH₃ | H | OCF₃ | H | H | H | 0 | 0 |
| 59 | Et | H | CF₃ | H | H | CH₃ | H | OCF₂CF₂H | H | H | H | 0 | 0 |
| 60 | Et | H | CF₃ | H | H | CH₃ | H | Br | OCF₃ | H | H | 0 | 0 |
| 61 | Et | H | CF₃ | H | H | CH₃ | H | H | SCH₃ | H | H | 0 | 0 |
| 62 | Et | H | CF₃ | H | H | CH₃ | H | H | OCH₃ | H | H | 0 | 0 |
| 63 | Et | H | CF₃ | H | H | CH₃ | H | CF₃ | CH₃ | H | H | 2 | 0 |
| 64 | Et | H | CF₃ | H | H | CH₃ | H | H | F | H | H | 0 | 0 |
| 65 | Et | H | CF₃ | H | H | CH₃ | H | H | F | H | H | 2 | 0 |
| 66 | Et | H | CF₃ | H | H | CH₃ | H | H | CN | H | H | 0 | 0 |

TABLE 31

| Compound | | | | | | | | | | | | | |
|---|---|---|---|---|---|---|---|---|---|---|---|---|---|
| of Present | | | | | | | | | | | | | |
| Invention | R¹ | R² | R³ | R⁴ | R⁵ | R⁶ | R⁷ | R⁸ | R⁹ | R¹⁰ | R¹¹ | n | Q |
| 67 | Et | H | CF₃ | H | H | CH₃ | H | H | CycHex | H | H | 0 | 0 |
| 68 | Et | H | CF₃ | H | H | CH₃ | H | Cl | CF₃ | H | H | 0 | 0 |
| 69 | Et | H | CF₃ | H | H | CH₃ | H | Cl | CF₃ | H | H | 2 | 0 |
| 70 | Et | H | CF₃ | H | H | CH₃ | H | H | CF₃ | H | F | 2 | 0 |
| 71 | Et | H | CF₃ | H | H | CH₃ | H | F | CF₃ | H | F | 2 | 0 |
| 72 | Et | H | CF₃ | H | H | CH₃ | H | CF₃ | F | H | H | 2 | 0 |
| 73 | Et | H | CF₃ | H | H | CH₃ | H | CF₃ | Cl | H | H | 2 | 0 |
| 74 | Et | H | CF₃ | H | H | CH₃ | H | CF₃ | Br | H | H | 2 | 0 |
| 75 | Et | H | CF₃ | H | H | CH₃ | H | CF₃ | OCH₃ | H | H | 2 | 0 |
| 76 | Et | H | CF₃ | H | H | CH₃ | H | OCF₃ | H | H | H | 2 | 0 |
| 77 | Et | H | CF₃ | H | H | CH₃ | H | OCF₂CF₂H | H | H | H | 2 | 0 |
| 78 | Et | H | CF₃ | H | H | CH₃ | H | Br | OCF₃ | H | H | 2 | 0 |
| 79 | Et | H | CF₃ | H | H | CH₃ | H | H | SO₂CH₃ | H | H | 2 | 0 |
| 80 | Et | H | CF₃ | H | H | CH₃ | H | H | OCH₃ | H | H | 2 | 0 |
| 81 | Et | H | CF₃ | H | H | CH₃ | H | H | OCH₂CH₃ | H | H | 2 | 0 |
| 82 | Et | H | CF₃ | H | H | CH₃ | H | H | OPh | H | H | 2 | 0 |
| 83 | Et | H | CF₃ | H | H | CH₃ | H | H | CN | H | H | 2 | 0 |
| 84 | Et | H | CF₃ | H | H | CH₃ | H | H | CycHex | H | H | 2 | 0 |
| 85 | Et | H | CF₃ | H | H | CH₃ | H | CF₃ | CF₃ | H | H | 2 | 0 |
| 86 | Et | H | CF₃ | H | H | CH₃ | H | F | CF₃ | F | H | 2 | 0 |
| 87 | Et | H | CO₂Et | H | H | CH₃ | H | H | SCF₃ | H | H | 2 | 0 |
| 88 | Et | H | Br | H | H | CH₃ | H | H | SCF₃ | H | H | 2 | 0 |

TABLE 32

| Compound | | | | | | | | | | | | | |
|---|---|---|---|---|---|---|---|---|---|---|---|---|---|
| of Present | | | | | | | | | | | | | |
| Invention | R¹ | R² | R³ | R⁴ | R⁵ | R⁶ | R⁷ | R⁸ | R⁹ | R¹⁰ | R¹¹ | n | Q |
| 89 | Et | H | CF₃ | H | H | CH₃ | H | CF₃ | H | H | H | 2 | 0 |
| 90 | Et | H | CN | H | H | CH₃ | H | H | SCF₃ | H | H | 2 | 0 |
| 91 | Et | H | CO₂H | H | H | CH₃ | H | H | SCF₃ | H | H | 2 | 0 |
| 92 | Et | H | CON(CH₃)₂ | H | H | CH₃ | H | H | SCF₃ | H | H | 2 | 0 |
| 93 | Et | H | CONH(CH₃) | H | H | CH₃ | H | H | SCF₃ | H | H | 2 | 0 |
| 94 | Et | H | CONH₂ | H | H | CH₃ | H | H | SCF₃ | H | H | 2 | 0 |
| 95 | Et | H | NHCO₂CH₃ | H | H | CH₃ | H | H | SCF₃ | H | H | 2 | 0 |
| 96 | Et | H | NHCO₂C(CH₃)₃ | H | H | CH₃ | H | H | SCF₃ | H | H | 2 | 0 |
| 97 | Et | H | NH₂ | H | H | CH₃ | H | H | SCF₃ | H | H | 2 | 0 |
| 98 | Et | H | CF₃ | H | H | CH₃ | H | F | CF₃ | H | F | 0 | 0 |
| 99 | Et | H | CF₃ | H | H | CH₃ | H | CF₃ | CF₃ | H | H | 0 | 0 |
| 100 | Et | H | CF₃ | H | H | CH₃ | H | F | CF₃ | F | H | 0 | 0 |
| 101 | Et | H | CF₃ | H | H | CH₃ | H | H | OCH₂CH₃ | H | H | 0 | 0 |
| 102 | Et | H | CF₃ | H | H | CH₃ | H | H | OPh | H | H | 0 | 0 |
| 103 | Et | H | CO₂Et | H | H | CH₃ | H | H | SCF₃ | H | H | 0 | 0 |

(In [Table 28] to [Table 32] above, Et represents an ethyl group, Pr represents a n-propyl group, CycPr represents a cyclopropyl group, CycHex represents a cyclohexyl group, and Ph represents a phenyl group.)
¹H-NMR data of the compound of the present invention shown in [Table 28] to [Table 32] are shown below.

### Compound of Present Invention 6

¹H-NMR (CDCl₃) δ: 7.65-6.94 (6H, m), 3.42 (3H, brs), 3.01 (2H, q), 1.35 (3H, t).

### Compound of Present Invention 7

¹H-NMR (CDCl₃) δ: 7.54-7.44 (1H, m), 7.40-7.11 (2H, m), 6.91-6.72 (2H, m), 6.69-6.57 (1H, m), 3.42 (3H, bra), 3.01 (2H, q), 1.34 (3H, t).

### Compound of Present Invention 8

¹H-NMR (CDCl₃) δ: 8.38-7.09 (7H, m), 3.70-3.18 (5H, m), 1.39-1.28 (3H, m).

### Compound of Present Invention 9

¹H-NMR (CDCl₃) δ: 8.38-7.03 (7H, m), 3.72-3.15 (5H, m), 1.38-1.29 (3H, m).

### Compound of Present Invention 10

¹H-NMR (CDCl₃) δ: 8.37-7.10 (7H, m), 3.72-3.17 (5H, m), 1.39-1.29 (3H, m).

### Compound of Present Invention 11

¹H-NMR (CDCl₃) δ: 8.48-6.95 (7H, m), 3.76-3.10 (5H, m), 1.47-1.30 (3H, m).

### Compound of Present Invention 12

¹H-NMR (CDCl₃) δ: 8.39-7.08 (7H, m), 3.71-3.19 (5H, m), 1.39-1.29 (3H, m).

### Compound of Present Invention 13

¹H-NMR (CDCl₃) δ: 8.38-6.59 (6H, m), 3.67-3.10 (5H, m), 1.40-1.25 (3H, m).

### Compound of Present Invention 14

¹H-NMR (CDCl₃) δ: 8.38-7.13 (6H, m), 3.68-3.11 (5H, m), 1.40-1.29 (3H, m).

### Compound of Present Invention 15

¹H-NMR (CDCl₃) δ: 8.38-7.13 (6H, m), 3.68-3.11 (5H, m), 1.40-1.29 (3H, m).

Compounds represented by the formula (M6).

### Compound of Present Invention 18

¹H-NMR (CDCl₃) δ: 8.69-6.79 (8H, m), 3.66-2.41 (5H, m), 1.45-1.03 (3H, m).

### Compound of Present Invention 19

¹H-NMR (CDCl₃) δ: 7.80-6.98 (8H, m), 3.54-2.48 (5H, m), 1.34-1.22 (3H, m).

### Compound of Present Invention 25

¹H-NMR (CDCl₃) δ: 8.48-6.95 (8H, m), 3.75-3.16 (5H, m), 1.38-1.26 (3H, m).

### Compound of Present Invention 26

¹H-NMR (CDCl₃) δ: 8.15-6.94 (8H, m), 3.71-3.17 (5H, m), 1.39-1.29 (3H, m).

### Compound of Present Invention 44

¹H-NMR (CDCl₃) δ: 7.94-6.93 (7H, m), 3.48 (3H, s), 3.04-2.93 (2H, m), 1.36-1.29 (3H, m).

### Compound of Present Invention 45

¹H-NMR (CDCl₃) δ: 8.35-7.14 (7H, m), 3.72-3.14 (5H, m), 1.38-1.26 (3H, m).

### Compound of Present Invention 46

¹H-NMR (CDCl₃) δ: 7.47-6.89 (7H, m), 3.53-3.11 (3H, m), 2.98 (2H, q), 2.41-2.19 (3H, m), 1.34 (3H, t).

### Compound of Present Invention 47

¹H-NMR (CDCl₃) δ: 8.39-6.90 (7H, m), 3.71-3.10 (5H, m), 2.46-2.17 (3H, m), 1.34 (3H, t).

### Compound of Present Invention 48

¹H-NMR (CDCl₃) δ: 8.09-6.86 (7H, m), 4.42-3.40 (3H, m), 3.26-2.86 (2H, m), 2.77-2.43 (2H, m), 1.40-1.26 (3H, m), 1.26-1.06 (3H, m).

### Compound of Present Invention 49

¹H-NMR (CDCl₃) δ: 8.37-8.14 (1H, m), 8.08-6.92 (6H, m), 4.45-3.14 (5H, m), 2.73-2.48 (2H, m), 1.40-1.08 (6H, m).

### Compound of Present Invention 50

¹H-NMR (CDCl₃) δ: 7.67-6.94 (7H, m), 4.42-2.72 (6H, m), 1.37-1.07 (9H, m).

### Compound of Present Invention 51

¹H-NMR (CDCl₃) δ: 8.35-8.16 (1H, m), 8.02-7.02 (6H, m), 4.40-3.16 (5H, m), 2.99-2.73 (1H, m), 1.37-1.11 (9H, m).

### Compound of Present Invention 52

¹H-NMR (CDCl₃) δ: 7.81-7.06 (6H, m), 3.62-2.86 (5H, m), 1.34 (3H, t).

### Compound of Present Invention 53

¹H-NMR (CDCl₃) δ: 7.83-6.82 (6H, m), 3.69-2.81 (5H, m), 1.33 (3H, t).

### Compound of Present Invention 54

¹H-NMR (CDCl₃) δ: 7.74-7.00 (6H, m), 3.46 (3H, s), 2.98 (2H, d), 1.33 (3H, t).

### Compound of Present Invention 55

¹H-NMR (CDCl₃) δ: 7.71-6.97 (6H, m), 3.46 (3H, s), 2.97 (2H, q), 1.32 (3H, t).

### Compound of Present Invention 56

¹H-NMR (CDCl₃) δ: 7.77-6.92 (6H, m), 3.60-2.88 (5H, m), 2.38 (3H, s), 1.33 (3H, t).

### Compound of Present Invention 57

¹H-NMR (CDCl₃) δ: 7.71-6.72 (6H, m), 3.98-2.89 (8H, m), 1.33 (3H, t).

### Compound of Present Invention 58

¹H-NMR (CDCl₃) δ: 7.48-6.92 (7H, m), 3.50 (3H, s), 2.98 (2H, q), 1.33 (3H, t).

### Compound of Present Invention 59

¹H-NMR (CDCl₃) δ: 7.64-6.89 (7H, m), 5.83 (1H, t), 3.50 (3H, s), 2.97 (2H, q), 1.33 (3H, t).

### Compound of Present Invention 60

¹H-NMR (CDCl₃) δ: 7.73-6.96 (6H, m), 3.44 (3H, s), 2.99 (2H, q), 1.34 (3H, t).

### Compound of Present Invention 61

¹H-NMR (CDCl₃) δ: 7.47-6.95 (7H, m), 3.47 (3H, s), 2.98 (2H, q), 2.40 (3H, s), 1.33 (3H, t).

### Compound of Present Invention 62

¹H-NMR (CDCl₃) δ: 7.40 (1H, s), 7.20 (1H, d), 7.13 (1H, d), 7.07 (2H, d), 6.68 (2H, d), 3.71 (3H, s), 3.46 (3H, s), 2.98 (2H, q), 1.33 (3H, t).

### Compound of Present Invention 63

¹H-NMR (DMSO-D₆) δ: 8.31-7.26 (6H, m), 3.61-3.35 (5H, m), 2.30 (3H, s), 1.22-1.16 (3H, m).

### Compound of Present Invention 64

¹H-NMR (CDCl₃) δ: 7.71-6.74 (7H, m), 3.55-2.89 (5H, m), 1.33 (3H, t).

### Compound of Present Invention 65

¹H-NMR (CDCl₃) δ: 8.36-6.85 (7H, m), 3.69-3.13 (5H, m), 1.38-1.28 (3H, m).

### Compound of Present Invention 66

¹H-NMR (CDCl₃) δ: 7.55 (2H, d), 7.47 (1H, s), 7.37-7.19 (4H, m), 3.46 (3H, s), 2.98 (2H, q), 1.33 (3H, t).

### Compound of Present Invention 67

¹H-NMR (CDCl₃) δ: 7.67-6.84 (7H, m), 3.55-2.89 (5H, m), 2.44-2.32 (1H, m), 1.90-1.08 (8H, m).

### Compound of Present Invention 68

¹H-NMR (CDCl₃) δ: 7.56 (1H, d), 7.50 (1H, s), 7.43 (1H, s), 7.37 (1H, d), 7.30-7.24 (1H, m), 7.22-7.15 (1H, m), 3.43 (3H, s), 3.00 (2H, q), 1.34 (3H, t).

### Compound of Present Invention 69

¹H-NMR (CDCl₃) δ: 8.37-7.16 (6H, m), 3.67-3.16 (5H, m), 1.39-1.29 (3H, m).

### Compound of Present Invention 70

¹H-NMR (CDCl₃) δ: 8.40-7.19 (6H, m), 3.70-3.13 (5H, m), 1.38-1.29 (3H, m).

### Compound of Present Invention 71

¹H-NMR (CDCl₃) δ: 8.37-8.19 (1H, m), 8.08-7.30 (4H, m), 3.61-3.12 (5H, m), 1.42-1.29 (3H, m).

### Compound of Present Invention 72

¹H-NMR (CDCl₃) δ: 8.37-6.99 (6H, m), 3.64-3.16 (5H, m), 1.40-1.29 (3H, m).

### Compound of Present Invention 73

¹H-NMR (CDCl₃) δ: 8.36-8.20 (1H, m), 8.05-7.15 (5H, m), 3.64-3.17 (5H, m), 1.38-1.29 (3H, m).

### Compound of Present Invention 74

¹H-NMR (CDCl₃) δ: 8.36-8.20 (1H, m), 8.06-7.15 (5H, m), 3.64-3.17 (5H, m), 1.39-1.29 (3H, m).

### Compound of Present Invention 75

¹H-NMR (DMSO-D₆) δ: 8.25-7.09 (6H, m), 3.96-3.36 (8H, m), 1.21-1.14 (3H, m).

### Compound of Present Invention 76

¹H-NMR (CDCl₃) δ: 8.38-8.18 (1H, m), 8.06-6.95 (6H, m), 3.69-3.17 (5H, m), 1.38-1.29 (3H, m).

### Compound of Present Invention 77

¹H-NMR (CDCl₃) δ: 8.37-8.16 (1H, m), 8.12-6.92 (6H, m), 6.09-5.67 (1H, m), 3.67-3.15 (5H, m), 1.38-1.29 (3H, m).

### Compound of Present Invention 78

¹H-NMR (CDCl₃) δ: 8.38-8.19 (1H, m), 8.11-7.06 (5H, m), 3.65-3.14 (5H, m), 1.38-1.29 (3H, m).

### Compound of Present Invention 80

¹H-NMR (CDCl₃) δ: 8.35-6.54 (7H, m), 3.86-3.12 (8H, m), 1.38-1.28 (3H, m).

### Compound of Present Invention 81

¹H-NMR (CDCl₃) δ: 8.36-6.53 (7H, m), 4.07-3.13 (7H, m), 1.44-1.28 (6H, m).

### Compound of Present Invention 82

¹H-NMR (CDCl₃) δ: 8.35-6.78 (12H, m), 3.67-3.15 (5H, m), 1.37-1.28 (3H, m).

### Compound of Present Invention 83

¹H-NMR (CDCl₃) δ: 8.37-7.09 (7H, m), 3.67-3.19 (5H, m), 1.39-1.29 (3H, m).

### Compound of Present Invention 84

¹H-NMR (CDCl₃) δ: 8.36-7.00 (7H, m), 3.68-3.14 (5H, m), 2.56-2.33 (1H, m), 1.93-1.14 (8H, m).

### Compound of Present Invention 85

¹H-NMR (CDCl₃) δ: 8.37-7.12 (6H, m), 4.03-3.21 (5H, m), 1.43-1.28 (3H, m).

### Compound of Present Invention 86

¹H-NMR (CDCl₃) δ: 8.36-7.05 (5H, m), 4.03-3.17 (5H, m), 1.41-1.30 (3H, m).

### Compound of Present Invention 89

¹H-NMR (CDCl₃) δ: 8.38-7.10 (7H, m), 3.68-3.18 (5H, m), 1.38-1.28 (3H, m).

### Compound of Present Invention 90

¹H-NMR (CDCl₃) δ: 8.41-7.04 (7H, m), 3.74-3.14 (5H, m), 1.37-1.28 (3H, m).

### Compound of Present Invention 92

¹H-NMR (CDCl₃) δ: 8.12-7.97 (1H, m), 7.87-7.02 (6H, m), 3.72-2.73 (11H, m), 1.36-1.29 (3H, m).

### Compound of Present Invention 93

¹H-NMR (CDCl₃) δ: 8.40-7.83 (2H, m), 7.77-7.08 (5H, m), 6.70-6.42 (1H, m), 3.71-3.17 (5H, m), 3.09-2.96 (3H, m), 1.35-1.28 (3H, m).

### Compound of Present Invention 95

¹H-NMR (CDCl₃) δ: 7.90-6.79 (7H, m), 3.86-3.16 (8H, m), 1.37-1.28 (3H, m).

### Compound of Present Invention 96

¹H-NMR (CDCl₃) δ: 7.89-6.61 (7H, m), 3.69-3.17 (5H, m), 1.56-1.46 (9H, m), 1.36-1.28 (3H, m).

### Compound of Present Invention 98

¹H-NMR (CDCl₃) δ: 7.64-7.14 (5H, m), 3.38 (3H, s), 3.02 (2H, q), 1.35 (3H, t).

### Compound of Present Invention 99

¹H-NMR (CDCl₃) δ: 8.06-7.27 (6H, m), 3.47 (3H, s), 2.98 (2H, q), 1.32 (3H, t).

### Compound of Present Invention 100

¹H-NMR (CDCl₃) δ: 8.08-6.92 (5H, m), 3.39 (3H, s), 3.02 (3H, q), 1.34 (3H, t).

### Compound of Present Invention 101

¹H-NMR (CDCl₃) δ: 7.39 (1H, s), 7.20 (1H, d), 7.13 (1H, d), 7.05 (2H, d), 6.67 (2H, d), 3.92 (2H, q), 3.46 (3H, s), 2.97 (2H, q), 1.38-1.30 (6H, m).

### Compound of Present Invention 102

¹H-NMR (CDCl₃) δ: 7.52-6.64 (12H, m), 3.55-3.13 (3H, m), 2.98 (2H, q), 1.32 (3H, t).

### Compound of Present Invention 103

¹H-NMR (CDCl₃) δ: 7.94-7.09 (7H, m), 4.35 (2H, q), 3.55-3.42 (3H, m), 2.98 (2H, q), 1.37 (3H, t), 1.31 (3H, t).

Compounds represented by the formula (M6):

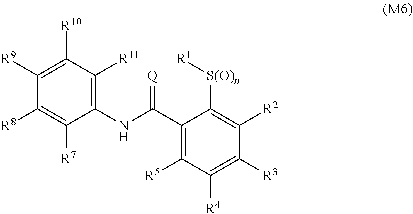

wherein R¹, R², R³, R⁴, R⁵, R⁷, R⁸, R⁹, R¹⁰, R¹¹, n and Q represent the combinations shown in [Table 33] to [Table 35] shown below.

TABLE 33

| Intermediate | | | | | | | | | | | | |
|---|---|---|---|---|---|---|---|---|---|---|---|---|
| Compound | R¹ | R² | R³ | R⁴ | R⁵ | R⁷ | R⁸ | R⁹ | R¹⁰ | R¹¹ | n | Q |
| M6-1 | Et | H | CF₃ | H | H | H | H | CF₃ | H | H | 0 | 0 |
| M6-2 | Et | H | CF₃ | H | H | H | H | OCF₃ | H | H | 0 | 0 |
| M6-3 | Et | H | CF₃ | H | H | H | H | CF₂CF₃ | H | H | 0 | 0 |
| M6-4 | Et | H | CF₃ | H | H | H | H | SCF₃ | H | H | 0 | 0 |
| M6-5 | Et | H | CF₃ | H | H | H | CF₃ | H | CF₃ | H | 0 | 0 |
| M6-6 | Et | H | CF₃ | H | H | H | Cl | H | Cl | H | 0 | 0 |
| M6-7 | Et | H | CF₃ | H | H | H | F | H | F | H | 0 | 0 |
| M6-8 | Et | H | CF₃ | H | H | H | H | CF₃ | H | H | 2 | 0 |
| M6-9 | Et | H | CF₃ | H | H | H | H | OCF₃ | H | H | 2 | 0 |
| M6-10 | Et | H | CF₃ | H | H | H | H | CF₂CF₃ | H | H | 2 | 0 |
| M6-11 | Et | H | CF₃ | H | H | H | H | SCF₃ | H | H | 2 | 0 |
| M6-12 | Et | H | CF₃ | H | H | H | CF₃ | H | CF₃ | H | 2 | 0 |
| M6-13 | Et | H | CF₃ | H | H | H | Cl | H | Cl | H | 2 | 0 |
| M6-14 | Et | H | CF₃ | H | H | H | F | H | F | H | 2 | 0 |
| M6-15 | Et | H | F | H | H | H | H | CF₂CF₃ | H | H | 2 | 0 |
| M6-16 | Et | H | Cl | H | H | H | H | CF₂CF₃ | H | H | 2 | 0 |
| M6-17 | Et | H | Br | H | H | H | H | CF₂CF₃ | H | H | 2 | 0 |
| M6-18 | Et | H | l | H | H | H | H | CF₂CF₃ | H | H | 2 | 0 |
| M6-19 | Et | H | 2-pyridyl | H | H | H | H | CF₂CF₃ | H | H | 2 | 0 |
| M6-20 | Et | H | 2-pyrimidyl | H | H | H | H | CF₂CF₃ | H | H | 2 | 0 |
| M6-21 | Et | H | CH₃ | H | H | H | H | CF₂CF₃ | H | H | 2 | 0 |
| M6-22 | Et | H | OCH₃ | H | H | H | H | CF₂CF₃ | H | H | 2 | 0 |

TABLE 34

| Intermediate | | | | | | | | | | | | |
|---|---|---|---|---|---|---|---|---|---|---|---|---|
| Compound | R¹ | R² | R³ | R⁴ | R⁵ | R⁷ | R⁸ | R⁹ | R¹⁰ | R¹¹ | n | Q |
| M6-23 | Et | H | H | H | H | H | H | SCF₃ | H | H | 0 | 0 |
| M6-24 | Et | H | H | H | H | H | H | CF₂CF₃ | H | H | 0 | 0 |
| M6-25 | Et | H | CF₃ | H | H | H | H | Br | H | H | 0 | 0 |
| M6-26 | Et | H | CF₃ | H | H | H | H | CH₃ | H | H | 0 | 0 |
| M6-27 | Et | H | CF₃ | H | H | H | H | CH₂CH₃ | H | H | 0 | 0 |
| M6-28 | Et | H | CF₃ | H | H | H | H | CH(CH₃)₂ | H | H | 0 | 0 |
| M6-29 | Et | H | CF₃ | H | H | H | H | F | H | H | 0 | 0 |
| M6-30 | Et | H | CF₃ | H | H | H | CF₃ | H | H | H | 2 | 0 |
| M6-31 | Et | H | CF₃ | H | H | H | Cl | CF₃ | H | H | 0 | 0 |
| M6-32 | Et | H | CF₃ | H | H | H | H | CF₃ | H | F | 0 | 0 |
| M6-33 | Et | H | CF₃ | H | H | H | F | CF₃ | H | F | 0 | 0 |
| M6-34 | Et | H | CF₃ | H | H | H | CF₃ | F | H | H | 0 | 0 |
| M6-35 | Et | H | CF₃ | H | H | H | CF₃ | Cl | H | H | 0 | 0 |
| M6-36 | Et | H | CF₃ | H | H | H | CF₃ | Br | H | H | 0 | 0 |
| M6-37 | Et | H | CF₃ | H | H | H | CF₃ | CH₃ | H | H | 0 | 0 |
| M6-38 | Et | H | CF₃ | H | H | H | CF₃ | OCH₃ | H | H | 0 | 0 |
| M6-39 | Et | H | CF₃ | H | H | H | OCF₃ | H | H | H | 0 | 0 |
| M6-40 | Et | H | CF₃ | H | H | H | OCF₃CF₂H | H | H | H | 0 | 0 |
| M6-41 | Et | H | CF₃ | H | H | H | Br | OCF₃ | H | H | 0 | 0 |
| M6-42 | Et | H | CF₃ | H | H | H | CF₃ | CF₃ | H | H | 0 | 0 |
| M6-43 | Et | H | CF₃ | H | H | H | F | CF₃ | F | H | 0 | 0 |
| M6-44 | Et | H | CF₃ | H | H | H | H | SCH₃ | H | H | 0 | 0 |

TABLE 35

| Intermediate | | | | | | | | | | | | |
|---|---|---|---|---|---|---|---|---|---|---|---|---|
| Compound | R¹ | R² | R³ | R⁴ | R⁵ | R⁷ | R⁸ | R⁹ | R¹⁰ | R¹¹ | n | Q |
| M6-45 | Et | H | CF₃ | H | H | H | H | OCH₃ | H | H | 0 | 0 |
| M6-46 | Et | H | CF₃ | H | H | H | H | OCH₂CH₃ | H | H | 0 | 0 |
| M6-47 | Et | H | CF₃ | H | H | H | H | OPh | H | H | 0 | 0 |
| M6-48 | Et | H | CF₃ | H | H | H | H | CN | H | H | 0 | 0 |
| M6-49 | Et | H | CF₃ | H | H | H | H | CycHex | H | H | 0 | 0 |

(In [Table 33] to [Table 35] above, Et represents an ethyl group, CycHex represents a cyclohexyl group, and Ph represents a phenyl group.)
¹H-NMR data of the intermediate compound of the present invention shown in [Table 33] to [Table 35] are shown below.

### Intermediate Compound M6-6

¹H-NMR (CDCl₃) δ: 8.70 (1H, s), 7.91 (1H, d), 7.70 (1H, s), 7.64 (2H, d), 7.59 (1H, d), 7.20-7.17 (1H, m), 3.01 (2H, q), 1.34 (3H, t).

### Intermediate Compound M6-7

¹H-NMR (CDCl₃) δ: 8.73 (1H, s), 7.91 (1H, d), 7.69 (1H, s), 7.58 (1H, d), 7.33-7.22 (2H, m), 6.69-6.58 (1H, m), 3.01 (2H, q), 1.34 (3H, t).

### Intermediate Compound M6-8

¹H-NMR (CDCl₃) δ: 9.04 (1H, s), 8.01 (1H, d), 7.92 (1H, s), 7.76 (1H, d), 7.58 (2H, d), 7.38 (2H, d), 3.54 (2H, q), 1.29 (3H, t).

### Intermediate Compound M6-9

¹H-NMR(CDCl₃) δ: 8.68 (1H, brs), 8.06-7.97 (2H, m), 7.77 (1H, d), 7.53 (2H, d), 7.06 (2H, d), 3.52 (2H, q), 1.29 (3H, t).

### Intermediate Compound M6-10

¹H-NMR(CDCl₃) δ: 9.09 (1H, brs), 8.01 (1H, d), 7.91 (1H, s), 7.75 (1H, d), 7.62 (2H, d), 7.37 (2H, d), 3.54 (2H, q), 1.29 (3H, t).

### Intermediate Compound M6-11

¹H-NMR(CDCl₃) δ: 9.09 (1H, s), 8.01 (1H, d), 7.91 (1H, s), 7.75 (1H, d), 7.62 (2H, d), 7.37 (2H, d), 3.54 (2H, q), 1.29 (3H, t).

### Intermediate Compound M6-12

¹H-NMR(CDCl₃) δ: 9.51 (1H, s), 8.02 (1H, d), 7.97 (2H, a), 7.85 (1H, s), 7.74 (1H, d), 7.43 (1H, s), 3.60 (2H, q), 1.32 (3H, t).

### Intermediate Compound M6-13

¹H-NMR (CDCl₃) δ: 9.17 (1H, s), 8.00 (1H, d), 7.79 (1H, s), 7.68 (1H, d), 7.43 (2H, d), 6.97 (1H, dd), 3.55 (2H, q), 1.28 (3H, q).

### Intermediate Compound M6-14

¹H-NMR (CDCl₃) δ: 9.16 (1H, s), 8.00 (1H, d), 7.80 (1H, s), 7.70 (1H, d), 7.08 (2H, dd), 6.48-6.36 (1H, m), 3.53 (2H, q), 1.27 (3H, t).

### Intermediate Compound M6-23

¹H-NMR (CDCl₃) δ: 9.11 (1H, s), 7.88 (1H, dd), 7.76 (2H, dd), 7.66 (2H, d), 7.51 (1H, dd), 7.45 (1H, td), 7.37 (1H, td), 2.95 (2H, q), 1.29 (3H, t).

### Intermediate Compound M6-24

¹H-NMR (CDCl₃) δ: 9.15 (1H, s), 7.89 (1H, dd), 7.83 (2H, d), 7.60 (2H, d), 7.51 (1H, dd), 7.45 (1H, td), 7.37 (1H, td), 2.95 (2H, q), 1.29 (3H, t).

### Intermediate Compound M6-25

¹H-NMR (CDCl₃) δ: 8.56 (1H, s), 7.90 (1H, d), 7.68 (1H, s), 7.60-7.55 (3H, m), 7.50 (2H, d), 3.00 (2H, q), 1.33 (3H, t).

### Intermediate Compound M6-26

¹H-NMR (CDCl₃) δ: 8.43 (1H, s), 7.88 (1H, d), 7.66 (1H, s), 7.58-7.51 (3H, m), 7.19 (2H, d), 3.00 (2H, q), 2.35 (3H, s), 1.32 (3H, t).

### Intermediate Compound M6-27

¹H-NMR (CDCl₃) δ: 8.44 (1H, s), 7.86 (1H, d), 7.66 (1H, s), 7.59-7.51 (3H, m), 7.21 (2H, d), 2.99 (2H, q), 2.65 (2H, q), 1.32 (3H, t), 1.24 (3H, t).

### Intermediate Compound M6-28

¹H-NMR (CDCl₃) δ: 8.41 (1H, s), 7.88 (1H, d), 7.66 (1H, s), 7.59-7.53 (3H, m), 7.26-7.22 (2H, m), 3.00 (2H, q), 2.96-2.88 (1H, m), 1.32 (3H, t), 1.26 (6H, d).

### Intermediate Compound M6-29

¹H-NMR (CDCl₃) δ: 8.49 (1H, s), 7.89 (1H, d), 7.67 (1H, s), 7.63 (2H, dd), 7.56 (1H, d), 7.09 (2H, dd), 3.01 (2H, q), 1.33 (3H, t).

### Intermediate Compound M6-30

¹H-NMR (CDCl₃) δ: 8.83 (1H, s), 8.03-7.94 (3H, m), 7.76 (1H, d), 7.50 (1H, d), 7.27-7.19 (2H, m), 3.55 (2H, q), 1.30 (3H, t).

### Intermediate Compound M6-31

¹H-NMR (CDCl₃) δ: 8.89 (1H, s), 7.95 (1H, s), 7.89 (1H, d), 7.70-7.66 (2H, m), 7.61 (1H, d), 7.56 (1H, d), 3.01 (2H, q), 1.33 (3H, t).

### Intermediate Compound M6-32

¹H-NMR(CDCl₃) δ: 9.16 (1H, s), 8.70 (1H, dd), 7.97 (1H, d), 7.72 (1H, s), 7.60 (1H, d), 7.49 (1H, d), 7.42 (1H, d), 3.03 (2H, q), 1.35 (3H, t).

### Intermediate Compound M6-33

¹H-NMR(CDCl₃) δ: 9.36 (1H, s), 8.57 (1H, dd), 7.99 (1H, d), 7.73 (1H, s), 7.61 (1H, d), 7.39 (1H, dd), 3.03 (2H, q), 1.35 (3H, t).

### Intermediate Compound M6-34

¹H-NMR(CDCl₃) δ: 8.70 (1H, s), 7.92 (2H, d), 7.87 (1H, d), 7.69 (1H, s), 7.58 (1H, d), 7.22 (1H, d), 3.02 (2H, q), 1.34 (3H, t).

### Intermediate Compound M6-35

¹H-NMR (CDCl₃) δ: 8.77 (1H, s), 7.98 (1H, s), 7.92 (1H, d), 7.87 (1H, d), 7.70 (1H, s), 7.59 (1H, d), 7.52 (1H, d), 3.02 (2H, q), 1.34 (3H, t).

### Intermediate Compound M6-36

¹H-NMR (CDCl₃) δ: 8.78 (1H, s), 7.98 (1H, s), 7.92 (1H, d), 7.80 (1H, d), 7.74-7.68 (2H, m), 7.59 (1H, d), 3.01 (2H, q), 1.34 (3H, t).

### Intermediate Compound M6-37

¹H-NMR (CDCl₃) δ: 8.62 (1H, s), 7.89 (1H, d), 7.84 (1H, s), 7.79 (1H, d), 7.68 (1H, s), 7.56 (1H, d), 7.30 (1H, d), 3.01 (2H, q), 2.48 (3H, brs), 1.33 (3H, t).

### Intermediate Compound M6-38

¹H-NMR (CDCl₃) δ: 8.54 (1H, s), 7.93-7.87 (2H, m), 7.76 (1H, d), 7.67 (1H, s), 7.56 (114, d), 7.03 (1H, d), 3.92 (3H, s), 3.01 (2H, q), 1.34 (3H, t).

### Intermediate Compound M6-39

¹H-NMR (CDCl₃) δ: 8.68 (1H, s), 7.92 (1H, d), 7.69 (2H, s), 7.58 (1H, d), 7.51 (1H, d), 7.40 (1H, dd), 7.05 (1H, d), 3.01 (2H, q), 1.34 (3H, t).

### Intermediate Compound M6-40

¹H-NMR (CDCl₃) δ: 8.67 (1H, s), 7.90 (1H, d), 7.68 (2H, s), 7.57 (1H, d), 7.50 (1H, d), 7.38 (1H, dd), 7.06 (1H, d), 5.92 (1H, t), 3.01 (2H, q), 1.33 (3H, t).

### Intermediate Compound M6-41

¹H-NMR (DMSO-D₆) δ: 10.84 (1H, s), 8.24 (1H, d), 7.76-7.72 (3H, m), 7.67 (1H, d), 7.56 (1H, dd), 3.08 (2H, q), 1.22 (3H, q).

### Intermediate Compound M6-42

¹H-NMR (CDCl₃) δ: 9.01 (1H, s), 8.12-8.05 (2H, m), 7.94 (1H, d), 7.87 (1H, d), 7.71 (1H, s), 7.60 (1H, d), 3.02 (2H, q), 1.34 (3H, t).

### Intermediate Compound M6-43

¹H-NMR (DMSO-D₆) δ: 11.18 (1H, s), 7.78-7.72 (2H, m), 7.68-7.60 (3H, m), 3.08 (2H, q), 1.24 (3H, t).

### Intermediate Compound M6-44

¹H-NMR (CDCl₃) δ: 8.51 (1H, s), 7.89 (1H, d), 7.67 (1H, s), 7.60 (2H, d), 7.55 (1H, d), 7.29 (2H, d), 3.00 (2H, q), 2.49 (3H, s), 1.32 (3H, t).

### Intermediate Compound M6-45

¹H-NMR (CDCl₃) δ: 8.38 (1H, s), 7.89 (1H, d), 7.67 (1H, s), 7.59-7.53 (3H, m), 6.92 (2H, d), 3.82 (3H, s), 3.00 (2H, q), 1.33 (3H, t).

### Intermediate Compound M6-46

¹H-NMR (CDCl₃) δ: 8.37 (1H, s), 7.88 (1H, d), 7.67 (1H, s), 7.58-7.52 (3H, m), 6.91 (2H, d), 4.04 (2H, q), 3.00 (2H, q), 1.42 (3H, t), 1.33 (3H, t).

### Intermediate Compound M6-47

¹H-NMR (CDCl₃) δ: 8.47 (1H, s), 7.90 (1H, d), 7.68 (1H, s), 7.62 (2H, d), 7.56 (1H, d), 7.37-7.31 (2H, m), 7.13-7.08 (1H, m), 7.07-6.99 (4H, m), 3.02 (2H, q), 1.34 (3H, t).

### Intermediate Compound M6-48

¹H-NMR (CDCl₃) δ: 8.87 (1H, s), 7.93 (1H, d), 7.80 (2H, d), 7.71-7.66 (3H, m), 7.59 (1H, d), 3.01 (2H, q), 1.34 (3H, t).

### Intermediate Compound M6-49

¹H-NMR (CDCl₃) δ: 8.40 (1H, s), 7.88 (1H, d), 7.67 (1H, s), 7.59-7.52 (3H, m), 7.23 (2H, d), 3.00 (2H, g), 2.55-2.45 (1H, m), 1.91-1.71 (5H, m), 1.48-1.20 (8H, m).

Next, formulation examples of the compound of the present invention are shown. The part means part by weight.

### Formulation Example 1

10 parts of any one of Compounds of Present Invention 1 to 103 and Intermediate Compounds M6-1 to M6-49 are dissolved in a mixture of 35 parts of xylene and 35 parts of DMF, and 14 parts of polyoxyethylenestyrylphenyl ether and 6 parts of calcium dodecylbenzenesulfonate are added thereto. The mixture is mixed to obtain each emulsifiable concentrate.

### Formulation Example 2

4 parts of sodium lauryl sulfate, 2 parts of calcium lignosulfonate, 20 parts of synthetic hydrous silicon oxide fine powder and 54 parts of diatomaceous earth are mixed, and 20 parts of any one of Compounds of Present Invention 1 to 103 and Intermediate Compounds M6-1 to M6-49 are further added thereto. The mixture is mixed to obtain each wettable powder.

### Formulation Example 3

1 part of synthetic hydrous silicon oxide fine powder, 2 parts of calcium lignosulfonate, 30 parts of bentonite and 65 parts of kaolin clay are added to 2 parts of any one of Compounds of Present Invention 1 to 103 and Intermediate Compounds M6-1 to M6-49. Subsequently, an appropriate amount of water is added to this mixture, and the mixture is further stirred, granulated with a granulator, and forced-air dried to obtain each granule.

### Formulation Example 4

1 part of any one of Compounds of Present Invention 1 to 103 and Intermediate Compounds M6-1 to M6-49 is dissolved in an appropriate amount of acetone, and 5 parts of synthetic hydrous silicon oxide fine powder, 0.3 parts of PAP and 93.7 parts of Fubasami clay are added thereto. The mixture is sufficiently stirred and mixed to evaporate and eliminate acetone to obtain each dust formulation.

### Formulation Example 5

35 parts of a mixture of polyoxyethylene alkyl ether sulfate ammonium salt and white carbon (weight ratio 1:1), 10 parts of any one of Compounds of Present Invention 1 to 103 and Intermediate Compounds M6-1 to M6-49 and 55 parts of water are mixed, and finely pulverized by wet grinding method to obtain each flowable.

### Formulation Example 6

0.1 parts of any one of Compounds of Present Invention 1 to 103 and Intermediate Compounds M6-1 to M6-49 are dissolved in 5 parts of xylene and 5 parts of trichloroethane, and the mixture is mixed with 89.9 parts of deodorized kerosene to obtain each oil solution.

### Formulation Example 7

10 mg of any one of Compounds of Present Invention 1 to 103 and Intermediate Compounds M6-1 to M6-49 is dissolved in 0.5 ml of acetone, and this solution is applied to 5 g of solid feed powder for animal (solid feed powder for breeding CE-2, product of CLEA Japan, Inc.), and the mixture is uniformly mixed. Subsequently, acetone is evaporated to dryness to obtain each poisonous bait.

### Formulation Example 8

An aerosol can is filled with 0.1 parts of any one of Compounds of Present Invention 1 to 103 and Intermediate Compounds M6-1 to M6-49 and 49.9 parts of Neothiazol (Chuo Kasei Co., Ltd.), and an aerosol valve is attached, and then the container is filled with 25 parts of dimethyl ether and 25 parts of LPG and shaken, and an actuator is attached to obtain an oil-based aerosol.

### Formulation Example 9

0.6 parts of any one of Compounds of Present Invention 1 to 103 and Intermediate Compounds M6-1 to M6-49, 0.01 parts of BHT (2,6-di-tert-butyl-4-methylphenol), 5 parts of xylene, 3.39 parts of deodorized kerosene and 1 part of emulsifier {Atmos 300 (registered trade name for Atmos Chemical Ltd.)} are mixed and dissolved, and an aerosol container is filled with the resulting solution and 50 parts of distilled water. A valve is attached to the container, and then the container is filled with 40 parts of a propellant (LPG) under pressure through the valve to obtain an aqueous aerosol.

### Formulation Example 10

0.1 g of any one of Compounds of Present Invention 1 to 103 and Intermediate Compounds M6-1 to M6-49 are dissolved in 2 ml of propylene glycol, and the solution is impregnated in a porous ceramic plate with a size of 4.0 cm×4.0 cm and 1.2 cm in thickness to obtain a heating type smoking agent.

### Formulation Example 11

5 parts of any one of Compounds of Present Invention 1 to 103 and Intermediate Compounds M6-1 to M6-49 and 95 parts of an ethylene-methyl methacrylate copolymer (a ratio of methyl methacrylate in the copolymer: 10% by weight, Acryft WD301, manufactured by SUMITOMO CHEMICAL Co., Ltd.) are melt-kneaded with a closed pressurizing kneader (manufactured by Moriyama Works), and the resulting kneaded matter is extruded from an extrusion molding machine through a molding die to obtain a rod-shaped molded body with a size of 15 cm in length and 3 mm in diameter.

### Formulation Example 12

5 parts of any one of Compounds of Present Invention 1 to 103 and Intermediate Compounds M6-1 to M6-49 and 95 parts of a soft vinyl chloride resin are melt-kneaded with a closed pressurizing kneader (manufactured by Moriyama Works), and the resulting kneaded matter is extruded from an extrusion molding machine through a molding die to obtain a rod-shaped molded body with a size of 15 cm in length and 3 mm in diameter.

### Formulation Example 13

100 mg of any one of 1 to 103 and Intermediate Compounds M6-1 to M6-49, 68.75 mg of lactose, 237.5 mg of corn starch, 43.75 mg of microcrystalline cellulose, 18.75 mg of polyvinylpyrrolidone, 28.75 mg of sodium carboxymethyl starch and 2.5 mg of magnesium stearate are mixed, and the resulting mixture is compressed to an appropriate size to obtain a tablet.

### Formulation Example 14

25 mg of any one of Compounds of Present Invention 1 to 103 and Intermediate Compounds M6-1 to M6-49, 60 mg of lactose, 25 mg of corn starch, 6 mg of carmellose calcium and an appropriate amount of 5% hydroxypropyl methylcellulose are mixed, and a hard shell gelatin capsule or a hydroxypropyl methylcellulose capsule is filled with the resulting mixture to obtain an encapsulated formulation.

### Formulation Example 15

Distilled water is added to 1000 mg of anyone of Compounds of Present Invention 1 to 103 and Intermediate Compounds M6-1 to M6-49, 500 mg of fumaric acid, 2000 mg of sodium chloride, 150 mg of methylparaben, 50 mg of propylparaben, 25000 mg of granulated sugar, 13000 mg of sorbitol (70% solution), 100 mg of Veegum K (Vanderbilt Co.), 35 mg of flavor and 500 mg of colorant, such that a final volume is 100 ml, and the mixture is mixed to obtain a suspension for oral administration.

### Formulation Example 16

5 parts of any one of Compounds of Present Invention 1 to 103 and Intermediate Compounds M6-1 to M6-49 and 5 parts of polysorbate 85 are dissolved in 3 parts of benzyl alcohol and 30 parts of propylene glycol, and a phosphate buffer is added to this solution so as to have a pH of 6.0 to 6.5, and then water is added until a total amount is 100 parts to obtain a liquid formulation for oral administration.

### Formulation Example 17

5 parts of aluminum distearate are dispersed in 57 parts of fractionated palm oil and 3 parts of polysorbate 85 by heating. This dispersion is cooled to room temperature, and 25 parts of saccharin are dispersed in an oily vehicle thereof. 10 parts of any one of Compounds of Present Invention 1 to 103 and Intermediate Compounds M6-1 to M6-49 are added thereto to obtain a paste formulation for oral administration.

### Formulation Example 18

5 parts of any one of Compounds of Present Invention 1 to 103 and Intermediate Compounds M6-1 to M6-49 and 95 parts of limestone filler are mixed, and a granule for oral administration is obtained using wet granulation method.

### Formulation Example 19

5 parts of any one of Compounds of Present Invention 1 to 103 and Intermediate Compounds M6-1 to M6-49 are dissolved in 80 parts of diethylene glycol monoethyl ether, and 15 parts of propylene carbonate are mixed therewith to obtain a spot-on solution.

### Formulation Example 20

10 parts of any one of Compounds of Present Invention 1 to 103 and Intermediate Compounds M6-1 to M6-49 are dissolved in 70 parts of diethylene glycol monoethyl ether, and 20 parts of 2-octyl dodecanol are mixed therewith to obtain a pour-on solution.

### Formulation Example 21

60 parts of NIKKOL TEALS-42 (Nikko Chemicals Co., Ltd., 42% aqueous solution of triethanolamine lauryl sulfate) and 20 parts of propylene glycol are added to 0.5 parts of any one of Compounds of Present Invention 1 to 103 and Intermediate Compounds M6-1 to M6-49, and the mixture is sufficiently stirred and mixed until it becomes a uniform solution, and then 19.5 parts of water are added and further sufficiently stirred and mixed to obtain a shampoo agent as a uniform solution.

### Formulation Example 22

0.15 parts of any one of Compounds of Present Invention 1 to 103 and Intermediate Compounds M6-1 to M6-49, 95 parts of an animal feed and 4.85 parts of a mixture of secondary calcium phosphate, diatomaceous earth, Aerosil and carbonate (or chalk) are sufficiently stirred and mixed to obtain a feed premix for animal.

### Formulation Example 23

7.2 g of any one of Compounds of Present Invention 1 to 103 and Intermediate Compounds M6-1 to M6-49 and 92.8 g of VOSCO S-55 (manufactured by Maruishi Pharmaceutical Co., Ltd.) are dissolved and mixed at 100° C., poured into a suppository mold, and cooled and solidified to obtain a suppository.

Next, the pest control effect of the compound of the present invention is shown as test examples.

### Test Example 1

Compounds of Present Invention 1 to 5, 8 to 15, 18, 19, 25, 26, 45, 52 to 55, 57 to 61, 64 to 66, 68 to 74, 78 and 86 to 90 and Intermediate Compounds M6-8, M6-9, M6-11, M6-14, and M6-23 as obtained in Formulation Example 5 were diluted with water, so as to have a concentration of the active ingredient of 500 ppm, to prepare a diluted liquid for test.

On the other hand, on a cucumber seedling (the first true leaf stage) planted in a plastic cup was inoculated with about 30 Aphis gossypii (whole stage), and leaving it for a day. 20 ml of the diluted liquid for test is sprayed on the seedling.

Six days after spraying, the number of surviving Aphis gossypii parasitized on the leaves of the cucumber was examined, and the controlling value was calculated according to the following equation:

Controlling value (%)={1−(Cb×Tai)/(Cai×Tb)}×100

wherein the symbols represent as follows:

Cb: the number of insects in a non-treated section before treatment,

Cai: the number of insects in a non-treated section on observation,

Tb: the number of insects in a treated section before treatment,

Tai: the number of insects in a treated section on observation,

wherein the non-treated section refers to a section where the diluted liquid for test prepared by diluting the formulation not containing the compound of the present invention as in Formulation Example 5 with the same amount of water as in the treated section was sprayed.

As a result, in the treated section using a diluted liquid for test of each of Compounds of Present Invention 1 to 5, 8 to 15, 18, 19, 25, 26, 45, 52 to 55, 57 to 61, 64 to 66, 68 to 74, 78, and 86 to 90 and Intermediate Compounds M6-8, M6-9, M6-11, M6-14 and M6-23, the controlling value was 90% or more.

### Test Example 2

The formulations of Compounds of Present Invention 4, 8 to 12, 18, 19, 25, 26, 71, 88 and 90 as obtained in Formulation Example 5 were diluted with water, so as to have a concentration of the active ingredient of 500 ppm, to prepare a diluted liquid for test.

On the other hand, a cucumber seedling (the second true leaf stage) planted in a plastic cup was drenched at its foot with 5 ml of the diluted liquid for test, and kept in a greenhouse at 25° C. for 7 days. On the cucumber leaf surface was inoculated about 30 Aphis gossypii (whole stage), and further kept in the greenhouse for 6 days, then the number of surviving Aphis gossypii parasitized on the leaves of the cucumber was examined, and the controlling value was calculated according to the following equation:

Controlling value (%)={1−(Cb×Tai)/(Cai×Tb)}×100

wherein the symbols represent as follows:

Cb: the number of insects in a non-treated section before treatment,

Cai: the number of insects in a non-treated section on observation,

Tb: the number of insects in a treated section before treatment,

Tai: the number of insects in a treated section on observation,

wherein the non-treated section refers to a section where the diluted liquid for test prepared by diluting the formulation not containing the compound of the present invention as in Formulation Example 5 with the same amount of water as in the treated section was sprayed.

As a result, in the treated section using a diluted liquid for test of each of Compounds of Present Invention 4, 8 to 12, 18, 19, 25, 26, 71, 88 and 90, the controlling value was 90% or more.

### Test Example 3

The formulations of Compounds of Present Invention 1 to 4, 6, 8 to 11, 18, 19, 53 to 55, 58, 68 and 71 and Intermediate Compound M6-23 as obtained in Formulation Example 5 were diluted with water, so as to have a concentration of the active ingredient of 500 ppm, to prepare a diluted liquid for test.

On a rice seedling in the second leaf stage planted in a polyethylene cup was sprayed 10 ml of each diluted liquid for test. After air-drying, 20 third-fourth instar larvae of Nilaparvata lugens were released, and kept in the greenhouse at 25° C. After 6 days, the number of Nilaparvata lugens parasitized on the rice was examined, and the controlling value was calculated according to the following equation:

Controlling value (%)={1−(Cb×Tai)/(Cai×Tb)}×100

wherein the symbols represent as follows:

Cb: the number of insects in a non-treated section before treatment,

Cai: the number of insects in a non-treated section on observation,

Tb: the number of insects in a treated section before treatment,

Tai: the number of insects in a treated section on observation,

wherein the non-treated section refers to a section where the diluted liquid for test prepared by diluting the formulation not containing the compound of the present invention as in Formulation Example 5 with the same amount of water as in the treated section was sprayed.

As a result, in the treated section using a diluted liquid for test of each of Compounds of Present Invention 1 to 4, 6, 8 to 11, 18, 19, 53 to 55, 58, 68 and 71 and Intermediate Compound M6-23, the controlling value was 90% or more.

### Test Example 4

The formulations of Compounds of Present Invention 8, 10, 12 and 18 as obtained in Formulation Example 5 were diluted with water, so as to have a concentration of the active ingredient of 500 ppm, to prepare a diluted liquid for test.

On the other hand, a rice seedling (2 weeks after sowing, the second leaf stage) planted in a plastic cup is drenched at its foot with 5 ml of each diluted liquid for test, and kept in a greenhouse of 25° C. for 7 days. 20 third-fourth instar larvae of Nilaparvata lugens were released, and further kept in the greenhouse for 6 days, then the number of surviving Nilaparvata lugens parasitized on the rice was examined, and the controlling value was calculated according to the following equation:

Controlling value (%)={1−(Cb×Tai)/(Cai×Tb)}×100

wherein the symbols represent as follows:

Cb: the number of insects in a non-treated section before treatment,

Cai: the number of insects in a non-treated section on observation,

Tb: the number of insects in a treated section before treatment,

Tai: the number of insects in a treated section on observation,

wherein the non-treated section refers to a section where the diluted liquid for test prepared by diluting the formulation not containing the compound of the present invention as in Formulation Example 5 with the same amount of water as in the treated section was sprayed.

As a result, in the treated section using a diluted liquid for test containing each of Compounds of Present Invention 8, 10, 12 and 18, the controlling value was 90% or more.

### Test Example 5

The formulations of Compounds of Present Invention 1 to 4, 8 to 12, 18, 19, 25, 26, 71, 87, 88 and 90 and Intermediate Compounds M6-11 and M6-23 as obtained in Formulation Example 5 were diluted with water, so as to have a concentration of the active ingredient of 500 ppm, to prepare a diluted liquid for test.

On the other hand, Bemisia tabaci adult was released on a tomato seedling (the third true leaf stage) planted in a polyethylene cup, and made to lay eggs for about 72 hours. The tomato seedling was kept in a greenhouse for 8 days, and when instar larvae hatched from the eggs, the above diluted liquid for test was sprayed in an amount of 20 ml/cup, and the cup was kept in a greenhouse at 25° C. After 7 days, the number of surviving instar larvae on the tomato leaves was examined, and the controlling value was calculated according to the following equation:

Controlling value (%)={1−(Cb×Tai)/(Cai×Tb)}×100

wherein the symbols represent as follows:

Cb: the number of insects in a non-treated section before treatment,

Cai: the number of insects in a non-treated section on observation,

Tb: the number of insects in a treated section before treatment,

Tai: the number of insects in a treated section on observation,

wherein the non-treated section refers to a section where the diluted liquid for test prepared by diluting the formulation not containing the compound of the present invention as in Formulation Example 5 with the same amount of water as in the treated section was sprayed.

As a result, in the treated section using a diluted liquid for test of each of Compounds of Present Invention 1 to 4, 8 to 12, 18, 19, 25, 26, 71, 87, 88 and 90 and Intermediate Compounds M6-11 and M6-23, the controlling value was 90% or more.

### Test Example 6

The formulations of Compounds of Present Invention 1 to 5, 8 to 11, 13, 14, 18, 19, 25, 26, 52 to 55, 57 to 61, 66, 68 to 74, 76 to 78, 86, 88 and 90 and Intermediate Compounds M6-8, M6-9, M6-11 to M6-13 and M6-23 to M6-24 as obtained in Formulation Example 5 were diluted with water, so as to have a concentration of the active ingredient of 500 ppm, to prepare a diluted liquid for test.

On the other hand, on cabbage at the third leaf stage planted in a polyethylene cup is sprayed in an amount of 20 mL/cup of the diluted liquid for test. After the drug solution was dried, the foliage part was cut off, and then placed in a 50 mL volume cup. Five second instar larvae of Plutella xylostella were released into the cup, and the cup was sealed with a lid. After the cup was kept at 25° C. for 5 days, the number of surviving insects was counted. The mortality was calculated according to the following equation:

Mortality=(Number of dead insects/Number of tested insects)×100.

As a result, in the treated section using a diluted liquid for test of each of Compounds of Present Invention 1 to 5, 8 to 11, 13, 14, 18, 19, 25, 26, 52 to 55, 57 to 61, 66, 68 to 74, 76 to 78, 86, 88 and 90 and Intermediate Compounds M6-8, M6-9, M6-11 to M6-13 and M6-23 to M6-24, the death rate was 80% or more.

### Test Example 7

The formulations of Compounds of Present Invention 1 to 5, 8 to 14, 18, 19, 25, 26, 54, 55, 60, 68 to 74, 76 to 78, 85, 86, 88 and 90 and Intermediate Compounds M6-8 to M6-13 as obtained in Formulation Example 5 were diluted with water, so as to have a concentration of the active ingredient of 500 ppm, to prepare a diluted liquid for test.

On the other hand, an apple tree was planted in a plastic cup, and grown until the seventh-eighth true leaf was spread. To the apple tree was sprayed in an amount of 20 mL/cup of the diluted liquid for test. After the drug solution was dried, 60 first-instar Adoxophyes orana fasciata were released, and the plastic cup the bottom of which was cut off and on which a filter paper was put was upside-down and covered. After 7 days, the number of surviving insects was counted, and the mortality was calculated according to the following equation:

Mortality (%)=(Number of dead insects/Number of tested insects)×100.

As a result, in the treated section using a diluted liquid for test of each of Compounds of Present Invention 1 to 5, 8 to 14, 18, 19, 25, 26, 54, 55, 60, 68 to 74, 76 to 78, 85, 86, 88 and 90 and Intermediate Compounds M6-8 to M6-13, the mortality was 90% or more.

As a result, in the treated section using a diluted liquid for test of each of Compounds of Present Invention 1 to 5, 10, 18, 19, 25, 26, 60 and 68 and Intermediate Compound M6-1, the mortality was 100%.

### Test Example 8

Each of formulations of Compounds of Present Invention 1 to 5, 10, 18, 19, 25, 26, 60 and 68 and Intermediate Compound M6-1 as obtained in Formulation Example 5 was diluted with water, so as to have a concentration of the active ingredient of 500 ppm, to prepare a diluted liquid for test.

A filter paper having a diameter of 5.5 cm was spread on the bottom of a polyethylene cup having the same diameter and 0.7 ml of the diluted liquid for test was added dropwise onto the filter paper, and 30 mg of sucrose was uniformly placed as bait. Into the polyethylene cup, 10 female imagoes of Musca domestica were released, and the cup was sealed with a lid. After 24 hours, the life and death of Musca domestica was examined, and the mortality was calculated.

As a result, in the treated section using a diluted liquid for test of each of Compounds of Present Invention 1 to 5, 10, 18, 19, 25, 26, 60 and 68 and Intermediate Compound M6-1, the mortality was 100%.

### Test Example 9

The formulations of Compounds of Present Invention 3, 10 and 11 as obtained in Formulation Example 5 were diluted with water, so as to have a concentration of the active ingredient of 500 ppm, to prepare a diluted liquid for test.

A filter paper having a diameter of 5.5 cm was spread on the bottom of a polyethylene cup having the same diameter and 0.7 ml of the diluted liquid for test was added dropwise onto the filter paper, and 30 mg of sucrose was uniformly placed as bait. Into the polyethylene cup, 2 male imagoes of Blattella germanica were released, and the cup was sealed with a lid. After 6 days, the life and death of Blattella germanica was examined, and the mortality was calculated.

As a result, in the treated section using a diluted liquid for test containing each of Compounds of Present Invention 3, 10 and 11, the mortality was 100%.

### Test Example 10

The formulations of Compounds of Present Invention 1 to 5, 8 to 11, 13, 18, 19, 25, 26, 44, 53 to 55, 59, 60, 68 to 74, 78, 86 to 88 and 90 and Intermediate Compounds M6-3, M6-4, M6-11, M6-12 and M6-23 as obtained in Formulation Example 5 were diluted with water, so as to have a concentration of the active ingredient of 500 ppm, to prepare a diluted liquid for test.

0.7 ml of the diluted liquid for test was added to 100 ml of ion-exchanged water (active ingredient concentration: 3.5 ppm). 20 last-instar larvae of Culex pipiens pallens were released into the solution. One day later, the life and death of the Culex pipiens pallens was examined, and the mortality of the pest was calculated.

As a result, in the treated section using a diluted liquid for test of each of Compounds of Present Invention 1 to 5, 8 to 11, 13, 18, 19, 25, 26, 44, 53 to 55, 59, 60, 68 to 74, 78, 86 to 88 and 90 and Intermediate Compounds M6-3, M6-4, M6-11, M6-12 and M6-23, the mortality was 95% or more.

### Test Example 11

2 mg of each of Compounds of Present Invention 15, 18, 19, 46, 54, 58, 62, 64, 69, 72, 77 to 79, 81 and 84 and Intermediate Compounds M6-9 to N16-10 was weighed in a screw tube (Maruemu No. 5; 27×55 mm), and 0.2 mL of acetone was added thereto and sealed with a cap to dissolve the compound. The screw tube was rotated and inverted to uniformity coat the drug solution onto the whole inner wall of the tube. After removing the cap, the solution was air-dried for about 2 hours, then non-blood-sucking nymphal ticks, Haemaphysalis longicornis (5 ticks/group) were released, and the tube was sealed with the cap. After 2 days, the number of dead ticks was examined, and the mortality was calculated according to the following equation:

Mortality (%)=100×(Number of dead ticks/Number of tested ticks).

As a result, in the treated section treated with each of Compounds of Present Invention 15, 18, 19, 46, 54, 58, 62, 64, 69, 72, 77 to 79, 81 and 84 and Intermediate Compounds M6-9 to M6-10, the mortality was 100%.

### INDUSTRIAL APPLICABILITY

The compound of the present invention has a controlling effect on pests and is useful as an active ingredient of a pest control agent.

1. An amide compound represented by formula (1): 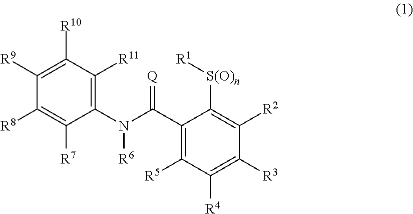
or an N-oxide thereof,
wherein
R¹ represents a C1 to C6 chain hydrocarbon group optionally having one or more atoms or groups selected from group X or a C3 to C6 cycloalkyl group optionally having one or more atoms or groups selected from group Y,
R², R³, R⁴ and R⁵ are the same or different and represent a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —S(O)₂NR¹²R¹³, —NR¹²R¹³, —NR¹²C(O)R¹³, —NR¹²CO₂R¹³, —NR¹²S(O)₂R¹⁴, —C(O)R¹², —CO₂R¹², —C(O)NR¹²R¹³, —SF₅, a cyano group, a nitro group, a halogen atom, or a hydrogen atom,
R⁶ represents a C1 to C6 chain hydrocarbon group optionally having one or more atoms or groups selected from group W, a C3 to C6 cycloalkyl group optionally having one or more atoms or groups selected from group Y, a C1 to C6 alkyl group having one phenyl group (wherein the phenyl group optionally has one or more atoms or groups selected from group Z), a C1 to C6 alkyl group having one 5- or 6-membered heterocyclic group (wherein the 5- or 6-membered heterocyclic group optionally has one or more atoms or groups selected from group Z), —C(O)R¹², —C(O)OR¹² or —C(O)NR¹²R¹³,
R⁷ and R¹¹ are the same or different and represent a C1 to C6 chain hydrocarbon group optionally having one or more atoms or groups selected from group X, a C3 to C6 cycloalkyl group optionally having one or more atoms or groups selected from group Y, —OR¹⁶, —S(O)ₘR¹⁶, a fluorine atom or a hydrogen atom,
R⁸, R⁹ and R¹⁰ are the same or different and represent a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —S(O)₂NR¹²R¹³, —NR¹²R¹³, —NR¹²C(O)R¹³, —NR¹²CO₂R¹³, —NR¹²S(O)₂R¹⁴, —C(O)R¹², —CO₂R¹⁴, —C(O)NR¹⁵R¹⁶, —SF₅, a cyano group, a nitro group, a halogen atom, or a hydrogen atom,
R¹² and R¹³ are the same or different and represent a C1 to C6 alkyl group optionally having one or more halogen atoms, a phenyl group optionally having one or more atoms or groups selected from group Z, a 5- or 6-membered heterocyclic group optionally having one or more atoms or groups selected from group Z or a hydrogen atom,
R¹⁴ represents a C1 to C6 alkyl group optionally having one or more halogen atoms, a phenyl group optionally having one or more atoms or groups selected from group Z or a 5- or 6-membered heterocyclic group optionally having one or more atoms or groups selected from group Z,
R¹⁵ represents a C1 to C6 alkyl group optionally having one or more halogen atoms or a hydrogen atom,
R¹⁶ represents a C1 to C6 alkyl group optionally having one or more halogen atoms,
Q represents an oxygen atom or a sulfur atom,
m represents 0, 1 or 2, and
n represents 0, 1 or 2;
group X is selected from the group consisting of C3 to C6 cycloalkyl groups optionally having one or more atoms or groups selected from the group consisting of halogen atoms and C1 to C3 alkyl groups, C1 to C6 alkoxy groups optionally having one or more halogen atoms, C1 to C6 alkylsulfanyl groups optionally having one or more halogen atoms, C1 to C6 alkylsulfinyl groups optionally having one or more halogen atoms, C1 to C6 alkylsulfonyl groups optionally having one or more halogen atoms, and halogen atoms;
group Y is selected from the group consisting of C1 to C6 alkyl groups optionally having one or more halogen atoms, C1 to C6 alkoxy groups optionally having one or more halogen atoms, C1 to C6 alkylsulfanyl groups optionally having one or more halogen atoms, C1 to C6 alkylsulfinyl groups optionally having one or more halogen atoms, C1 to C6 alkylsulfonyl groups optionally having one or more halogen atoms, and halogen atoms;
group Z is selected from the group consisting of C1 to C6 alkyl groups optionally having one or more halogen atoms, C1 to C6 alkoxy groups optionally having one or more halogen atoms, C1 to C6 alkylsulfanyl groups optionally having one or more halogen atoms, C1 to C6 alkylsulfinyl groups optionally having one or more halogen atoms, C1 to C6 alkylsulfonyl groups optionally having one or more halogen atoms, C1 to C6 alkylamino groups optionally having one or more halogen atoms, C2 to C8 dialkylamino groups optionally having one or more halogen atoms, C2 to C6 alkylcarbonyl groups optionally having one or more halogen atoms, C2 to C6 alkoxycarbonyl groups optionally having one or more halogen atoms, hydroxy groups, thiol groups, amino groups, cyano groups, nitro groups, and halogen atoms;
group W is selected from the group consisting of C3 to C6 cycloalkyl groups optionally having one or more atoms or groups selected from the group consisting of halogen atoms and C1 to C3 alkyl groups, C1 to C6 alkoxy groups optionally having one or more halogen atoms, C1 to C6 alkylsulfanyl groups optionally having one or more halogen atoms, C1 to C6 alkylsulfinyl groups optionally having one or more halogen atoms, C1 to C6 alkylsulfonyl groups optionally having one or more halogen atoms, C2 to C6 alkoxycarbonyl groups optionally having one or more halogen atoms, hydroxy groups, cyano groups, and halogen atoms;
wherein R⁸, R⁹ and R¹⁰ do not simultaneously represent a hydrogen atom, when R⁷, R⁸, R⁹ and R¹¹ represent a hydrogen atom, R¹⁰ does not represent a methyl group, and when R⁷, R⁸, R¹⁰ and R¹¹ represent a hydrogen atom, R⁹ does not represent a pyrrolidin-1-yl group, and
when m is 1 or 2 in —S(O)ₘR¹², R¹² does not represent a hydrogen atom.

2. The compound according to claim 1,
wherein R¹ is a C3 to C6 cycloalkyl group optionally having one or more atoms or groups selected from the group consisting of halogen atoms and C1 to C3 alkyl groups,
a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms, or
a C2 to C6 alkynyl group optionally having one or more halogen atoms,
R², R⁴ and R⁵ are the same or different and are a C1 to C6 alkyl group optionally having one or more halogen atoms, —OR¹², —S(O)ₘR¹², a halogen atom, or a hydrogen atom,
R³ is a C1 to C6 chain hydrocarbon group optionally having one or more atoms or groups selected from group X, a C3 to C6 cycloalkyl group optionally having one or more atoms or groups selected from group Y, a phenyl group optionally having one or more atoms or groups selected from group Z, a 5- or 6-membered heterocyclic group optionally having one or more atoms or groups selected from group Z, —OR¹², —S(O)ₘR¹², —NR¹²R¹³, —NR¹²C(O)R¹³, —NR¹²CO₂R¹³, —NR¹²S(O)₂R¹⁴, —C(O)R¹², —CO₂R¹², —C(O)NR¹²R¹³, —SF₅, a cyano group, a nitro group, a halogen atom, or a hydrogen atom,
R⁶ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms, a C2 to C6 alkynyl group optionally having one or more halogen atoms, a C1 to C6 alkyl group having one thiazolyl group (wherein the thiazolyl group optionally has one or more atoms or groups selected from the group consisting of C1 to C3 alkyl groups optionally having a halogen atom, C1 to C3 alkoxy groups optionally having a halogen atom, and halogen atoms), a C1 to C6 alkyl group having one pyridyl group (wherein the pyridyl group optionally has one or more atoms or groups selected from the group consisting of C1 to C3 alkyl groups optionally having a halogen atom, C1 to C3 alkoxy groups optionally having a halogen atom, and halogen atoms), a (C1 to C6 alkoxy)C1 to C6 alkyl group optionally having one or more halogen atoms, —C(O)R¹² or —C(O)OR¹²,
R⁷ and R¹¹ are the same or different and are a C1 to C6 alkyl group optionally having one or more halogen atoms, —OR¹⁶, —S(O)ₘR¹⁶, a fluorine atom or a hydrogen atom,
R⁸, R⁹ and R¹⁰ are the same or different and are a C1 to C6 chain hydrocarbon group optionally having one or more atoms or groups selected from group X, —OR¹², —S(O)ₘR¹², —SF₅, a halogen atom or a hydrogen atom,
R¹² and R¹³ are the same or different and are a C1 to C6 alkyl group optionally having one or more halogen atoms, a phenyl group (wherein the phenyl group optionally has a C1 to C3 alkyl group optionally having one or more halogen atoms or one or more halogen atoms), a 5- or 6-membered heterocyclic group (wherein the 5- or 6-membered heterocyclic group optionally has a C1 to C3 alkyl group optionally having one or more halogen atoms or one or more halogen atoms) or a hydrogen atom,
R¹⁴ is a C1 to C6 alkyl group optionally having one or more halogen atoms, a phenyl group (wherein the phenyl group optionally has a C1 to C3 alkyl group optionally having one or more halogen atoms or one or more halogen atoms) or a 5- or 6-membered heterocyclic group (wherein the 5- or 6-membered heterocyclic group optionally has a C1 to C3 alkyl group optionally having one or more halogen atoms or one or more halogen atoms), and
Q is an oxygen atom.

3. The compound according to claim 1,
wherein R¹ is a C3 to C6 cycloalkyl group optionally having one or more atoms or groups selected from the group consisting of halogen atoms and C1 to C3 alkyl groups,
a C2 to C6 alkyl group optionally having one or more halogen atoms or
a (C3 to C6 cycloalkyl)C1 to C3 alkyl group optionally having one or more halogen atoms,
R², R⁴ and R⁵ are the same or different and are a halogen atom or a hydrogen atom,
R³ is a C1 to C6 alkyl group optionally having one or more halogen atoms, a C2 to C6 alkenyl group optionally having one or more halogen atoms, a C2 to C6 alkynyl group optionally having one or more halogen atoms, a 5- or 6-membered heterocyclic group (wherein the 5- or 6-membered heterocyclic group optionally has a C1 to C3 alkyl group optionally having one or more halogen atoms or one or more halogen atoms), —OR¹², —S(O)ₘR¹², —NR¹²R¹³, —NR¹²C(O)R¹³, —NR¹²CO₂R¹³, —NR¹²S(O)₂R¹⁴, —C(O)R¹², —CO₂R¹², —C(O)NR¹²R¹³, a halogen atom, or a hydrogen atom,
R⁶ is a C1 to C6 alkyl group optionally having one or more atoms or groups selected from the group consisting of halogen atoms and a cyclopropyl group (wherein the cyclopropyl group optionally has one or more halogen atoms or one or more C1 to C3 alkyl groups), a C2 to C6 alkenyl group optionally having one or more halogen atoms or a C2 to C6 alkynyl group optionally having one or more halogen atoms,
R⁷ and R¹¹ are the same or different and are a fluorine atom or a hydrogen atom,
R⁸, R⁹ and R¹⁰ are the same or different and are a C1 to C6 alkyl group optionally having one or more halogen atoms, a C2 to C6 alkenyl group optionally having one or more halogen atoms, a C2 to C6 alkynyl group optionally having one or more halogen atoms, —OR¹², —S(O)ₘR¹², a halogen atom or a hydrogen atom,
R¹² and R¹³ are the same or different and are a C1 to C6 alkyl group optionally having one or more halogen atoms or a hydrogen atom,
R¹⁴ is a C1 to C6 alkyl group optionally having one or more halogen atoms, and
Q is an oxygen atom.

4. The compound according to claim 1, wherein R², R⁴ and R⁵ are a hydrogen atom.

5. The compound according to claim 1, wherein R⁷ and R¹¹ are a hydrogen atom.

6. The compound according to claim 1, wherein R³ is a C1 to C3 alkyl group optionally having one or more halogen atoms, a C1 to C3 alkoxy group optionally having one or more halogen atoms, a C1 to C3 alkylsulfanyl group optionally having one or more halogen atoms, a C1 to C3 alkylsulfinyl group optionally having one or more halogen atoms, a C1 to C3 alkylsulfonyl group optionally having one or more halogen atoms, a C1 to C6 alkylamino group optionally having one or more halogen atoms, a C2 to C8 dialkylamino group optionally having one or more halogen atoms, a C2 to C6 alkylcarbonylamino group optionally having one or more halogen atoms, a C2 to C6 alkoxycarbonylamino group optionally having one or more halogen atoms, a C1 to C3 alkylsulfonylamino group optionally having one or more halogen atoms, an amino group, a C2 to C6 alkylcarbonyl group optionally having one or more halogen atoms, a C2 to C6 alkoxycarbonyl group optionally having one or more halogen atoms, a C2 to C6 alkylaminocarbonyl group optionally having one or more halogen atoms, a C3 to C8 dialkylaminocarbonyl group optionally having one or more halogen atoms, an aminocarbonyl group, a pyridyl group (wherein the pyridyl group optionally has one or more atoms or substituents selected from the group consisting of C1 to C3 alkyl groups optionally having a halogen atom, C1 to C3 alkoxy groups optionally having a halogen atom, and halogen atoms), a pyrimidinyl group (wherein the pyrimidinyl group optionally has one or more atoms or substituents selected from the group consisting of C1 to C3 alkyl groups optionally having a halogen atom, C1 to C3 alkoxy groups optionally having a halogen atom, and halogen atoms), a halogen atom, or a hydrogen atom.

7. The compound according to claim 1, wherein R⁶ is a C1 to C6 alkyl group optionally having one or more atoms or groups selected from the group consisting of halogen atoms and a cyclopropyl group.

8. The compound according to claim 1, wherein R¹ is an ethyl group, a cyclopropyl group, or a cyclopropylmethyl group.

9. The compound according to claim 1, wherein R⁸, R⁹ and R¹⁰ are the same or different and are a C1 to C6 haloalkyl group, a C2 to C6 haloalkenyl group, a C1 to C6 haloalkoxy group, a C1 to C6 haloalkylsulfanyl group, a C1 to C6 haloalkylsulfinyl group, a C1 to C6 haloalkylsulfonyl group, a halogen atom, or a hydrogen atom.

10. The compound according to claim 1, wherein R⁸, R⁹ and R¹⁰ are the same or different and are a C1 to C3 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.

11. The compound according to claim 1, wherein R⁷, R⁸, R¹⁰ and R¹¹ are a hydrogen atom, R⁹ is a C1 to C3 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, or a halogen atom.

12. The compound according to claim 1, wherein R⁷, R⁹ and R¹¹ are a hydrogen atom, R⁸ and R¹⁰ are the same or different and are a C1 to C3 perfluoroalkyl group, a C1 to C3 perfluoroalkoxy group, a C1 to C3 perfluoroalkylsulfanyl group, a C1 to C3 perfluoroalkylsulfinyl group, a C1 to C3 perfluoroalkylsulfonyl group, a halogen atom, or a hydrogen atom.

13. A pest control composition comprising the compound as defined in claim 1, and an inert carrier.

14. A method for controlling pests comprising applying an effective amount of the compound as defined in claim 1 to a pest or a pest-infested area.
